(19) United States
(12) Patent Application Publication
(10) Pub. No.: US 2025/0002591 A1
(43) Pub. Date: Jan. 2, 2025

(54) INHIBITOR OF TYPE 1 INTERFERON RECEPTOR STEROID SPARING IN SYSTEMIC LUPUS ERYTHERMATOSUS PATIENTS

(71) Applicant: ASTRAZENECA AB, SODERTAJLE (SE)

(72) Inventors: GABRIEL ABREU, SODERTALJE (SE); RAJENDRA TUMMALA, WILMINGTON, DE (US)

(21) Appl. No.: 18/559,907

(22) PCT Filed: May 11, 2022
(86) PCT No.: PCT/EP2022/062770

§ 371 Date: Nov. 9, 2023

Related U.S. Application Data
(60) Provisional application No. 63/187,485, filed on May 12, 2021.
(60) Provisional application No. 63/230,113, filed on Aug. 6, 2021.

(51) Int. Cl. C07K 16/28; A61K 31/573; A61K 39/00; A61K 45/06; A61P 37/06

(52) U.S. Cl. CPC C07K 16/2866; A61K 31/573; A61K 45/06; A61P 37/06; A61K 2039/505; A61K 2039/545; C07K 2317/565

(57) ABSTRACT

The disclosure relates to methods and compositions for the treatment of Systemic Lupus Erythematosus (SLE).

### CROSS REFERENCE TO RELATED APPLICATIONS

This present application is a U.S. national stage application under 35 U.S.C. § 371 of International Application No. PCT/EP2022/062770, filed May 11, 2022, which claims priority to both U.S. Provisional Application No. 63/187,485, filed May 12, 2021 and U.S. Provisional Application No. 63/230,113, filed Aug. 6, 2021, the entire contents of which are incorporated herein by reference in their entirety.

### REFERENCE TO AN ELECTRONIC SEQUENCE LISTING

The instant application contains an electronic Sequence Listing that has been submitted electronically and is hereby incorporated by reference in its entirety. The sequence listing was created on May 11, 2022, is named “IFNAR790WOPCT-seql-000001.txt” and is 19,933 bytes in size.

### 1 BACKGROUND

### 1.1 Systemic Lupus Erythematosus (SLE)

Systemic lupus erythematosus (SLE) is a chronic, multisystemic, disabling autoimmune rheumatic disease of unknown aetiology. There is substantial unmet medical need in the treatment of SLE, particularly in subjects with moderate or severe disease. Long-term prognosis remains poor for many subjects.

A significant problem associated with the treatment of SLE, is the heterogeneous clinical manifestations of SLE¹. Any organ may be affected in SLE, with the skin, joints, and kidneys being the most commonly involved²⁻⁴. Incomplete disease control leads to progressive organ damage, poor quality of life, and increased mortality, with approximately half of all patients with SLE developing organ damage within 10 years of diagnosis^{5,6}. There remains the need for a medical intervention that improves SLE disease activity across multiple systems.

Clinical manifestations of SLE include, but are not limited to, constitutional symptoms, alopecia, rashes, serositis, arthritis, nephritis, vasculitis, lymphadenopathy, splenomegaly, haemolytic anaemia, cognitive dysfunction and other nervous system involvement. Increased hospitalisations and side effects of medications including chronic oral corticosteroids (OCS) and other immunosuppressive treatments add to disease burden in SLE⁷⁻⁹.

All of the therapies currently used for the treatment of SLE have well known adverse effect profiles and there is a medical need to identify new targeted therapies, particularly agents that may reduce the requirement for corticosteroids and cytotoxic agents. There has been only 1 new treatment (belimumab) for SLE approved by the US Food and Drug Administration (FDA) and European Medicines Agency (EMA) in the approximately 50 years since hydroxychloroquine was approved for use in discoid lupus and SLE. However, belimumab is not approved everywhere, and the uptake has been modest. Many agents currently used to treat SLE, such as azathioprine, cyclophosphamide, and mycophenolate mofetil/mycophenolic acid, have not been approved for the disease. Furthermore, these drugs all have well-documented safety issues and are not effective in all patients for all manifestations of lupus. Antimalarial agents (e.g. hydroxychloroquine) and corticosteroids may be used to control arthralgia, arthritis, and rashes. Other treatments include nonsteroidal anti-inflammatory drugs (NSAIDs); analgesics for fever, arthralgia, and arthritis; and topical sunscreens to minimise photosensitivity. It is often difficult to taper subjects with moderate or severe disease completely off corticosteroids, which cause long-term morbidity and may contribute to early cardiovascular mortality^{8,10}. Even small daily doses of 5 to 10 mg prednisone used long-term carry increased risks of side effects such as cataracts, osteoporosis, and coronary artery disease⁸.

### 1.2 Steroids

Glucocorticoids remain the mainstay treatment for SLE with doses varying depending on severity of disease manifestation. There is no “safe” dose of oral glucocorticoids in relation to the risk for development of glucocorticoid-induced damage such as cataracts, osteoporosis and coronary artery disease, and whereas higher glucocorticoid-exposure has been shown to be associated with increased overall damage accrual, fairly low to moderate doses can also be related to increased damage.

Glucocorticoids are the most commonly used therapy for patients with SLE owing to their immunosuppressant and anti-inflammatory properties, which reduce disease activity and prevent flares. Up to 80% of patients with SLE are exposed to glucocorticoids, with the majority being treated long-term. Although it may provide short-term efficacy, the frequent or maintenance use of oral glucocorticoid therapy carries a significant burden of toxicity that can independently contribute to morbidity and mortality and can adversely affect health-related quality of life. Therefore, novel, effective, and long-term treatments for SLE are needed to both reduce overall disease activity and glucocorticoid use.

### 1.3 the Challenge of Finding a Treatment for SLE

The clinical development of a new drug is a lengthy and costly process with low odds of success. For molecules that enter clinical development, less than 10% will eventually be approved by health regulatory authorities¹¹. Furthermore, the early clinical development of biotherapeutics is much lengthier than for small molecules.

Phase II trials are conducted in a small number of volunteers who have the disease of interest. They are designed to test safety, pharmacokinetics, and pharmacodynamics. A phase II trial may offer preliminary evidence of drug efficacy. However, the small number of participants and primary safety concerns within a phase II trial usually limit its power to establish efficacy. A Phase III trial is required to demonstrate the efficacy and safety of a clinical candidate. Critically, many clinical candidates that have shown promise at Phase II fail at Phase III. More than 90% of novel therapeutics entering Phase I trials fail during clinical development, primarily because of failure in efficacy or safety. The probability of success at phase III, following successful Phase II, is less than 50%¹².

The process of drug development is particularly difficult for SLE. This is because SLE is an especially complex and poorly understood disease. Not only is our understanding of the genetics of SLE rudimentary, but our insight into pathogenesis of most of the clinical manifestations are still relatively limited compared to other disease.

The complexity of SLE presents those wishing to develop new therapeutics with the problem of a patient population with extensive inhomogeneity¹³. This makes protocol design for clinical trials in SLE even more difficult, for example, as regards to the choice of inclusion criteria and primary and secondary endpoints. It is further difficult to predict the disease course in each patient. This inevitably increases the background noise that reduces the statistical power of a trial. A high placebo response rate limits the range in which the tested new drug can show an efficacy signal, making clinical trials even more difficult to conduct and interpret.

The difficulty in developing effective therapeutics for SLE leads to an even higher failure rate of therapeutics in this area in clinical trials, compared to therapeutics for other indications. The development of novel therapeutics for the treatment of SLE has thus proved extremely difficult. There are many examples of clinical candidates that showed promise at Phase II but failed to show efficacy and/or safety in subsequent Phase or Phase III trials.

### 1.4 Tabalumab

Tabalumab (LY2127399) is a human IgG4 monoclonal antibody that binds both soluble and membrane-bound B-cell activating factor (BAFF). The efficacy and safety of tabalumab was assessed in two 52-week, phase III, multicentre randomized, double-blind, placebo-controlled trial in patients with moderate-to-severe SLE (ILLUMINATE-1 and ILLUMINATE-2). The primary endpoint was proportion of patients achieving SLE Responder Index 5 (SRI-5) response at week 52. In ILLUMINATE-1 (NCT01196091), the primary endpoint was not met. Key secondary efficacy endpoints (OCS sparing, time to severe flare, worst fatigue in the last 24 hours) also did not achieve statistical significance, despite pharmacodynamic evidence of tabalumab biological activity (significant decreases in anti-dsDNA, total B-cells, and immunoglobulins)¹⁴. The primary endpoint was met in ILLUMINATE-2 (NCT01205438) in the higher dose group (tabalumab 120 mg every 2 weeks). However, no secondary endpoints were met, including OCS sparing¹⁵. Following ILLUMINATE-1 and ILLUMINATE-2, tabalumab development was suspended given the small effect size and inability to meet other important clinical endpoints.

### 1.5 Blisibimod

Blisibimod is a fusion protein composed of four BAFF-binding domains fused to the N-terminal Fc fragment of human IgG1 lg. Blisibimod for the treatment of SLE had promising Phase II results but was unsuccessful in Phase III. In a phase 2 double-blind, randomized, placebo-controlled clinical trial (PEARL-SC), patients with serologically active SLE and SELENA-SLEDAI score ≥6 points were randomized to 3 different doses of blisibimod or placebo (NCT01162681). At week 24, the highest dose group (200 mg once weekly) had a significantly higher SRI-5 response rate than the placebo group¹⁶. However, in a subsequent placebo-controlled, phase III randomized, double-blind study (CHABLIS-SC1) conducted on seropositive SLE patients with persistent high disease activity (SELENA-SLEDAI ≥10 points) the primary endpoint (SRI-6) was not met (NCT01395745). The secondary end points (SRI-4 and SRI-8) were also not reached 17

### 1.6 Atacicept

Atacicept (TACI-Ig) is a fully human recombinant fusion protein that neutralizes both BAFF and APRIL. The efficacy of atacicept for the treatment of SLE was evaluated in two phase II/III placebo randomized controlled trials (APRIL-LN and APRIL-SLE). The APRIL-LN trial compared renal response to atacicept versus placebo plus standard of care (newly initiated MMF and glucocorticoids) in patients with SLE nephritis. The trial was discontinued after serious adverse events were reported. In APRIL-SLE the primary end point, defined as a significantly decreased proportion of patients who developed a new flare from BILAG A or BILAG B domain scores, was not met in the lower dose (75 mg) arm (NCT00624338). Treatment of patients with the higher dose (150 mg) arm was discontinued due to serious AEs¹⁸.

### 1.7 Abetimus

Abetimus (LJP 394) comprises four synthetic oligodeoxynucleotides attached to a triethyleneglycol backbone, where more than 97% of these oligonucleotides are derived from dsDNA. The drug was designed to neutralize anti-dsDNA antibodies. In a double-blind, placebo-controlled study in SLE patients, treatment with LJP 394 in patients with high-affinity antibodies to its DNA epitope prolonged the time to renal flare, decreased the number of renal flares¹⁹. However, in a subsequent Phase III trial (NCT00089804) using higher doses of abetimus, with a primary endpoint of time to renal flare, study and further drug development was discontinued when interim analysis failed to show efficacy²⁰.

### 1.8 Rituximab

Rituximab is a chimeric anti-CD20 monoclonal antibody. Rituximab is an effective treatment in a number of autoimmune diseases, including rheumatoid arthritis and ANCA vasculitis. A small number of uncontrolled trials in lupus nephritis suggested that rituximab could also be potentially effective in patients with lupus nephritis. Efficacy and safety of rituximab was assessed in a randomized, double-blind, placebo-controlled phase III trial in patients with lupus nephritis treated concomitantly with mycophenolate mofetil (MMF) and corticosteroids (LUNAR) (NCT00282347). Rituximab therapy did not improve clinical outcomes after 1 year of treatment²¹. The efficacy and safety of rituximab in patients with moderate to severe SLE was evaluated in a multicentre placebo randomized controlled phase II/III trial (EXPLORER). The study randomized patients with baseline active SLE (defined as ≥1 new BILAG A scores or ≥2 BILAG B scores) to rituximab or placebo. The primary endpoint was the proportion of rituximab versus placebo-treated patients achieving a complete clinical response (CCR), partial clinical response (PCR), or no response at week 52. The primary endpoint was not met, with similar rates of complete and partial responses in rituximab and placebo arms at 52 weeks. Differences in time to first moderate or severe flare and change in HRQOL were also not significant²².

### 1.9 Abatacept

Abatacept is a CTLA-4 fusion protein that binds to CD80/86 on the surface of antigen presenting cells and blocks signalling through CD-28 required for T-cell activation. In preclinical studies abatacept was demonstrated to have immunomodulatory activity in the NZB/NZW murine model of lupu²³. Abatacept for treatment of non-renal SLE was been evaluated in a phase IIb, randomized, double-blind, placebo-controlled trial²⁴ (NCT00119678). The primary end point was the proportion of patients with new flare (adjudicated) according to a score of A/B on the British Isles Lupus Assessment Group (BILAG) index after the start of the steroid taper. The primary and secondary end points were not met.

### 1.10 Epratuzumab

Epratuzumab is a monoclonal antibody that modulates B-cell activity by binding CD22 on the surface of mature B-cells. Epratuzumab initially demonstrated efficacy in treating SLE at phase II trial but this was not confirmed in a follow-up second phase IIb trial or the subsequent phase III trial. Two phase IIb trials assessed the efficacy of epratuzumab with a BILAG-based primary endpoint in patients with moderate-to-severe SLE (ALLEVIATE 1 and 2). A trend towards clinical efficacy was observed and the primary end point was met by more patients treated with epratuzumab than placebo. Epratuzumab treatment also led to improvements in Health-related quality of life (HRQOL) and mean glucocorticoid dose²⁵. In another phase IIb trial (EMBLEM), patients with moderate-to-severe SLE were randomized to one of five epratuzumab doses or placebo. BICLA response at 12 weeks, the primary endpoint, was greater with all doses of epratuzumab than placebo, but the effect was not statistically significant. In the subsequent multicentre phase III trials EMBODY 1 and EMBODY 2, patients with moderate-to-severe SLE, the primary efficacy endpoint, BICLA response at 48 weeks, was not met. No significant differences were seen in secondary endpoints such as total SLEDAI-2K score, PGA, or mean glucocorticoid dose²⁶.

### 1.11 PF-04236921

PF-04236921 is a monoclonal antibody that binds soluble IL-6, a cytokine that is elevated in SLE patients. The efficacy of PF-0436921 was evaluated in a phase II RCT of patients with active SLE (BUTTERFLY) (NCT01405196). Patients were randomized to receive either subcutaneous PF-04236921 10 mg, 50 mg, or 200 mg or placebo every 8 weeks; the 200 mg dose arm was discontinued early because of 3 deaths. The primary efficacy endpoint was SRI-4 response at 24 weeks, with BICLA as a secondary endpoint. The primary endpoint was not met²⁷.

### 1.12 Belimumab

Belimumab is an anti-BAFF antibody approved for the treatment of SLE patients. Belimumab remains the only new treatment for SLE approved by the US Food and Drug Administration (FDA) and the European Medicines Agency (EMA) for approximately 60 years. Belimumab is also the only biologic approved for the treatment of SLE. However, belimumab does not permit steroid sparing, as evaluated by three phase 3, multicenter, double-blind, 52-week studies in adult patients with active SLE (BLISS-52, BLISS-76 and BLISS-SC)²⁸⁻³⁰. In these trials, sustained steroid sparing in patients receiving belimumab (IV or SC) did not achieve statistical significance²⁸⁻³⁰. For example, in patients receiving >7.5 mg/day of prednisone at baseline, only 18-19% of belimumab 10 mg/kg recipients were able to reduce their prednisone dose by ≥25% to ≤7.5 mg/day for 12 weeks, compared with 12-13% of placebo recipients²⁸. In a post-hoc analysis for the BLISS-52 and BLISS-76 data sets overall exposure to all corticosteroids actually increased on average for both the belimumab and placebo treatment groups³¹.

### 1.13 Type I IFN and Anifrolumab

Anifrolumab (MEDI-546) is a human immunoglobulin G1 kappa (IgG1_{K}) monoclonal antibody (mAb) directed against subunit 1 of the type I interferon receptor (IFNAR1). It is composed of 2 identical light chains and 2 identical heavy chains, with an overall molecular weight of approximately 148 kDa. Anifrolumab inhibits binding of type I IFN to type I interferon receptor (IFNAR) and inhibits the biologic activity of all type I IFNs.

Type I interferons (IFNs) are cytokines that have been implicated in SLE pathogenesis based on the finding of increased IFN-stimulated gene expression in most patients with SLE. In the phase 3 TULIP-2 trial of anifrolumab in patients with moderate to severe SLE, treatment response (assessed using British Isles Lupus Assessment Group [BILAG]-based Composite Lupus Assessment [BICLA]) was achieved by significantly more patients receiving anifrolumab compared with placebo at Week 52³². Similar results with this composite endpoint were observed in the phase 2 MUSE and phase 3 TULIP-1 trials^{33,34}. Importantly, composite endpoints used in SLE trials, such as BICLA and the SLE responder index (SRI), dichotomize changes in disease activity across different organ domains into a binary responder versus nonresponder result. While helpful for definitive demonstration of efficacy, this approach limits the ability to interpret treatment efficacy across the many organ domains that potentially affect patients with SLE.

### 1.14 Conclusion

There is a huge unmet need for an SLE therapy with a better efficacy and safety profile the currently available therapies^{35,36}. As described above, a large number and broad range of different biologics have been proposed and subjected to clinical trials, but these trials have failed to meet clinical meaningful endpoints in pivotal studies. Initial promise at Phase II of many proposed therapeutics was not translated into significant and meaningful clinical effect in subsequent pivotal Phase III clinical trials. Furthermore, there is a need for an SLE therapy that is efficacious across multiple organ domains. Furthermore, even approved treats for SLE do not permit steroid tapering in many patients.

Thus, there remains the need for safe and effective treatment of SLE that has proven clinical benefit, for example in a phase III double-blind, randomized, placebo controlled trial³⁷. SLE is a very heterogeneous disease and there further remains the need for a treatment of SLE manifestations that is effective across multiple organ systems, including musculoskeletal, mucocutaneous and immunologic domains.

The present invention solves one or more of the above-mentioned problems.

### 2 SUMMARY

The present invention relates to a method for steroid-sparing in a subject in need thereof, comprising administering to a subject a therapeutically effective amount of a type I IFN receptor (IFNAR1) inhibitor and a steroid, wherein the dose of the steroid administered to the subject is tapered from a pre-sparing dose at baseline to a post-sparing dose, wherein the subject has systemic lupus erythematosus (SLE).

The invention also relates to a method for treating SLE in a subject in need thereof, comprising administering a therapeutically effective amount of a IFNAR1 inhibitor to the subject, wherein treatment reduces or prevents increased administration of a steroid to the subject. The invention also relates to a method for treating SLE in a subject in need thereof, comprising administering a therapeutically effective amount of a IFNAR1 inhibitor to the subject, wherein treatment reduces or prevents increased administration of a steroid to the subject.

The invention also relates to a method for treating SLE in a subject in need thereof, comprising administering to the subject a therapeutically effective amount of a type I IFN receptor (IFNAR1) inhibitor, wherein the method does not comprise administering a steroid to the subject.

The invention is supported inter alia by data presented for the first time herein, including post hoc analysis of the phase 2 MUSE trial and the phase 3 TULIP-1 and TULIP-2 trials (NCT01438489, NCT02446912 and NCT02446899 respectively). The data show that, compared with placebo, treatment with a IFNAR1 inhibitor in patients SLE permits sparing of the steroid dose given to the patient, whilst simultaneously treating SLE associated disease. The data further show that treatment with the IFNAR1 inhibitor prevents an increase in the steroid dose given to SLE patients, compared to placebo. Furthermore, an IFNAR1 inhibitor is shown to reduce steroid associated organ damage and to increase the weight of underweight SLE patients.

### 3 BRIEF DESCRIPTION OF THE DRAWINGS

FIG. 1: Distribution of IFN transcript scores

FIG. 2: Changes to glucocorticoid dose by sustained glucocorticoid taper response in TULIP-1 and TULIP-2 (pooled data)

Glucocorticoid AUC through Week 52 for sustained glucocorticoid taper responders and nonresponders. The mean cumulative dose of glucocorticoids during the 52 weeks of treatment was 44% lower among patients who were glucocorticoid taper responders vs nonresponders. Error bars represent SE. Sustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day. AUC, area under the curve; SE, standard error.

FIG. 3: PRO response at Week 52 by sustained glucocorticoid taper response in TULIP-1 and TULIP-2 (pooled data)

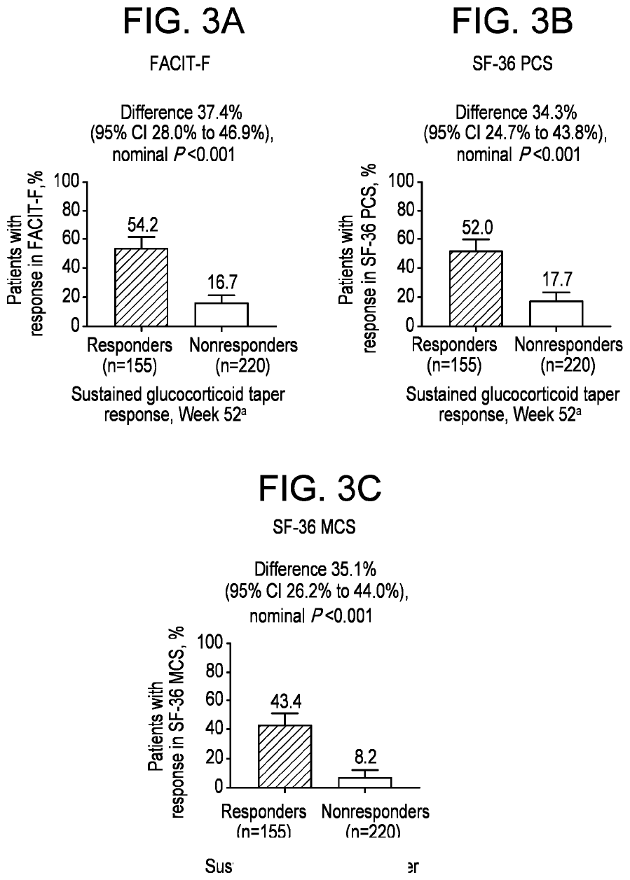

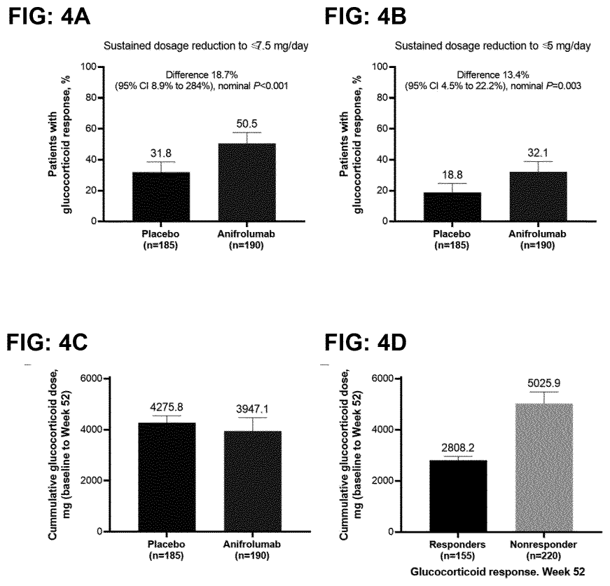

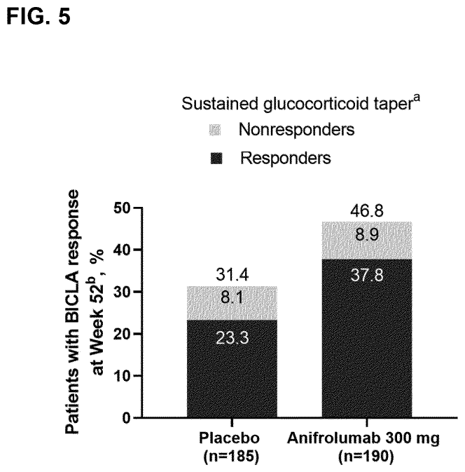

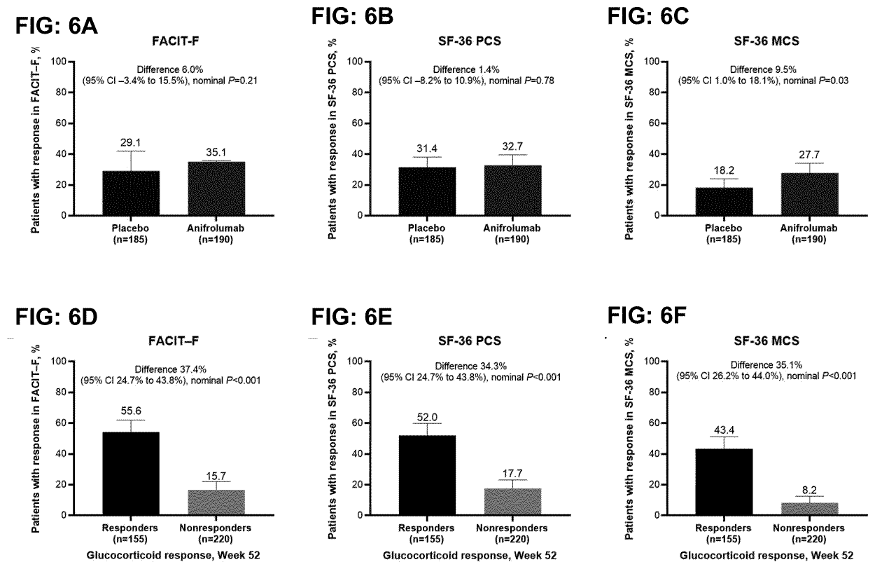

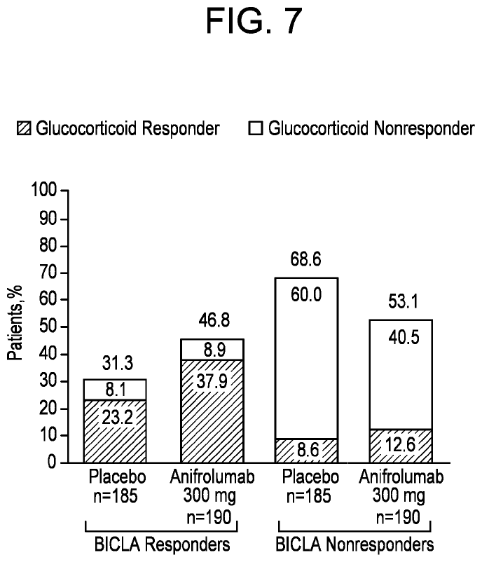

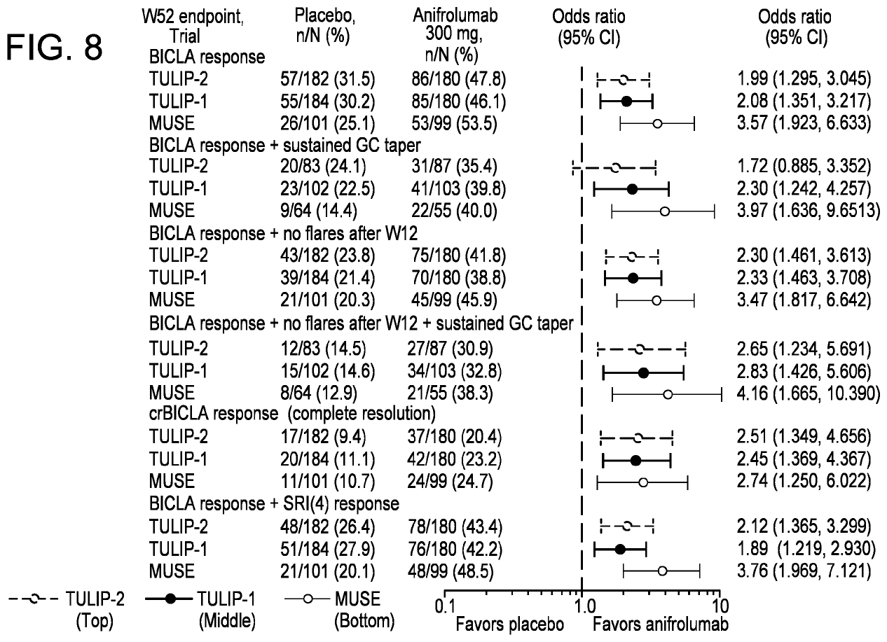

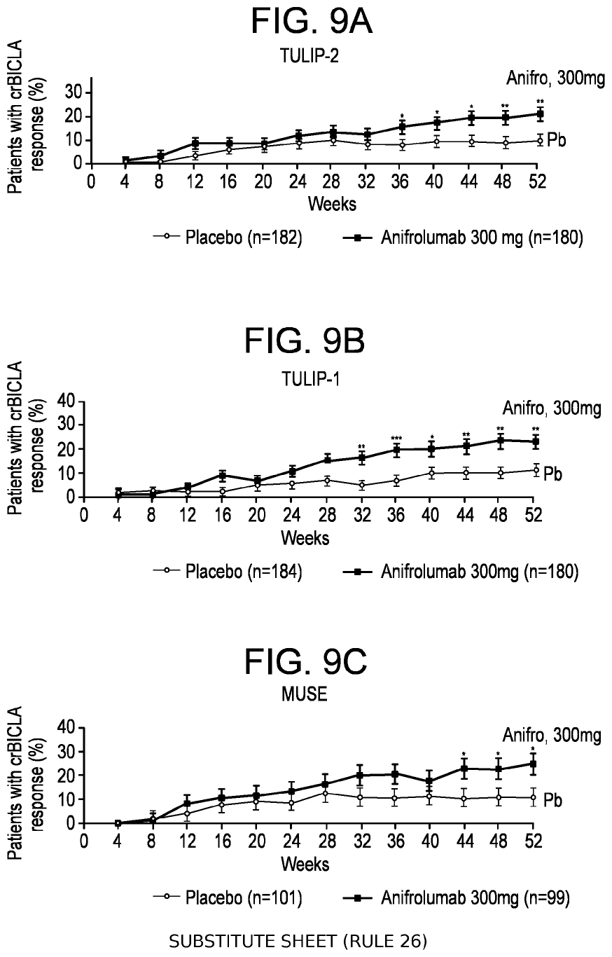

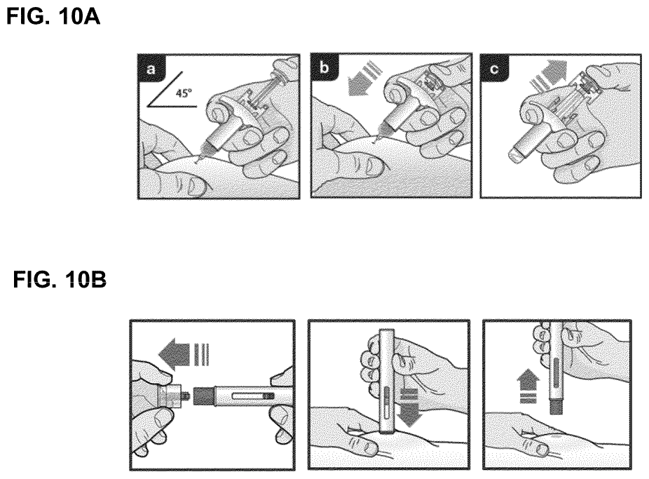

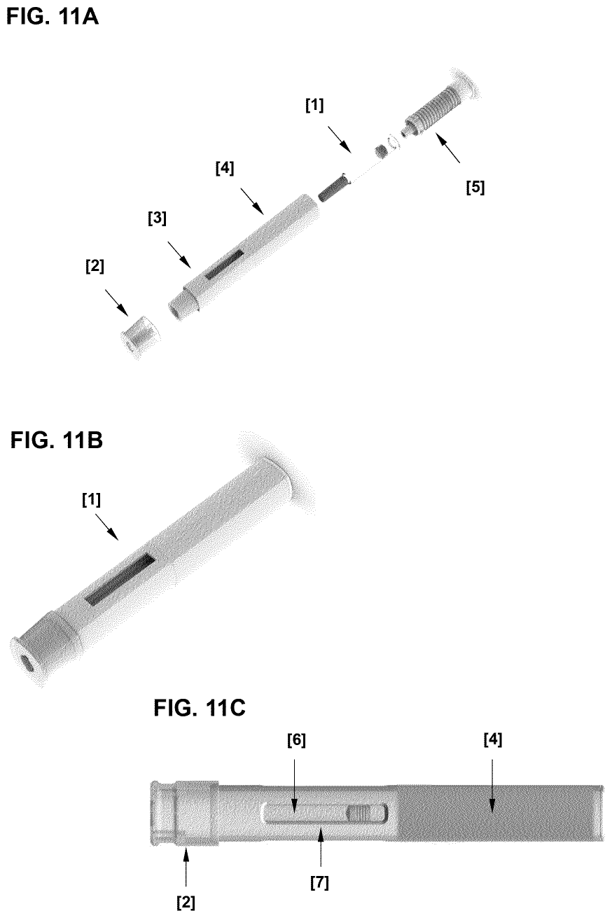

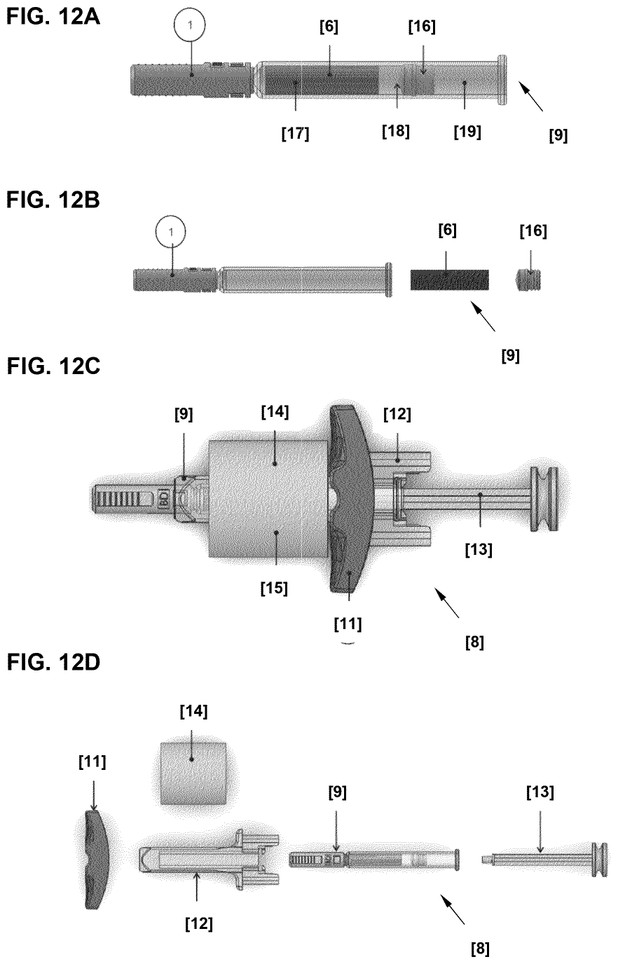

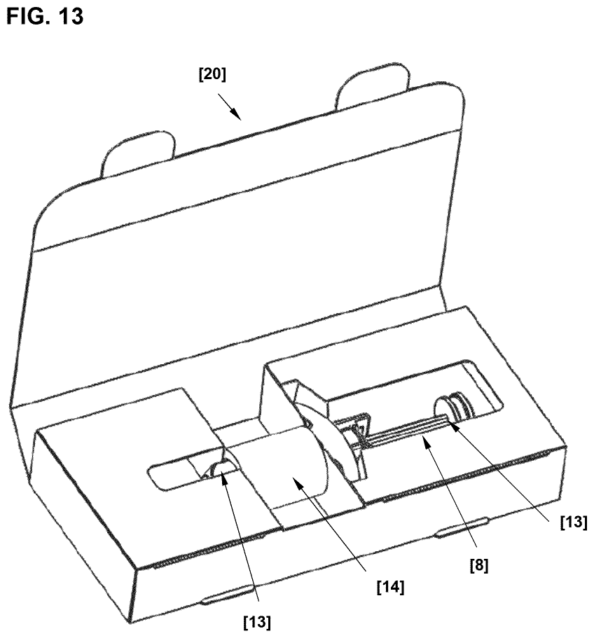

The sustained glucocorticoid taper responder group had more patients with clinically meaningful improvements in FACIT-F, SF-36 PCS, and SF-36 MCS scores (all P<0.001) compared with nonresponders. Patients with response in (FIG. 3A) FACIT-F, defined as an improvement from baseline to Week 52 >3; (FIG. 3B) SF-36 PCS, defined as an increase from baseline to Week 52 >3.4 in the PCS domain; and (FIG. 3C) SF-36 MCS, defined as an increase from baseline to Week 52 >4.6 in the MCS domain. FIG. 3A-C, Error bars represent 95% CI. Response rates, 95% Cls, and nominal P-values were calculated using a stratified Cochran-Mantel-Haenszel approach.

CI, confidence interval; FACIT-F, Functional Assessment of Chronic Illness Therapy-Fatigue; MCS, mental component summary; PCS, physical component summary; PRO, patient-reported outcome; SF-36, Short Form 36 Health Survey. ªSustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day.

FIG. 4: Glucocorticoid response and changes to dosage in patients receiving glucocorticoid ≥10 mg/day at baseline in TULIP-1 and TULIP-2 (pooled data)

Using a more stringent threshold of glucocorticoid reduction to ≤5 mg/day, more patients also achieved sustained glucocorticoid reductions to ≤5 mg/day from Weeks 40 to 52 with anifrolumab compared with placebo. Patients achieving sustained oral glucocorticoid dosage reduction to ≤7.5 mg/day (FIG. 4A) and ≤5 mg/day at Week 52 (FIG. 4B). Error bars represent 95% Cl.

The mean cumulative dose of glucocorticoids during the 52 weeks of treatment was 8% lower in the anifrolumab group vs the placebo group and 44% lower among patients who were glucocorticoid responders vs nonresponders. FIG. 4C: Oral glucocorticoid AUC through Week 52 per treatment group. Error bars represent SE. FIG. 4D: Oral glucocorticoid AUC through Week 52 for glucocorticoid responders and nonresponders. Error bars represent SE. Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

AUC, area under the curve; CI, confidence interval; LS, least squares; SE, standard error.

FIG. 5: Sustained glucocorticoid taper response in patients categorized by BICLA response at Week 52 in patients receiving glucocorticoid ≥10 mg/day at baseline in TULIP-1 and TULIP-2 (pooled data)

A total of 46.8% (89/190) of patients treated with anifrolumab and receiving baseline glucocorticoids ≥10 mg/day achieved a BICLA response at Week 52 versus 31.4% (58/185) of patients who received placeb.

BICLA, British Isles Lupus Assessment Group-based Composite Lupus Assessment; BILAG-2004, British Isles Lupus Assessment Group 2004; PtGA, Patient's Global Assessment; SLEDAI-2K, Systemic Lupus Erythematosus Disease Activity Index 2000; VAS, visual analogue scale. ^{a}Sustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day. ^{b}BICLA response defined as reduction of all baseline BILAG-2004 A and B scores and no worsening in other organ systems, no worsening from baseline in SLEDAI-2K, and no increase ≥0.3 points on a 3-point PtGA VAS from baseline.

FIG. 6: PRO response at Week 52 in patients receiving glucocorticoid ≥10 mg/day at baseline in TULIP-1 and TULIP-2 (pooled data)

Treatment with anifrolumab, compared with placebo, resulted in more patients with nominally significant improvement in SF-36 MCS scores (P=0.03), but not SF-36 PCS or FACIT-F. The glucocorticoid responders group had more patients with nominally significant improvements in all PROs (all P<0.001) compared with nonresponders. Patients with response in FACIT-F, defined as an improvement from baseline to Week 52 >3 (FIG. 6A, FIG. 6D); SF-36 PCS, defined as an increase from baseline to Week 52 >3.4 in the PCS domain (FIG. 6B, FIG. 6E); and SF-36 MCS, defined as an increase from baseline to Week 52 >4.6 in the MCS domain (FIG. 6C, FIG. 6F). FIG. 6A-F, Error bars represent 95% CI. Response rates, Cls, and nominal P-values were calculated using a stratified Cochran-Mantel-Haenszel approach.

CI, confidence interval; FACIT-F, Functional Assessment of Chronic Illness Therapy-Fatigue; MCS, mental component summary; PCS, physical component summary; PRO, patient-reported outcome; SF-36, Short Form 36 Health Survey. Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

FIG. 7: Sustained glucocorticoid taper response in patients categorized by BICLA response at Week 52 in patients receiving glucocorticoid ≥10 mg/day at baseline in TULIP-1 and TULIP-2 (pooled data)

BICLA, British Isles Lupus Assessment Group-based Composite Lupus Assessment; BILAG-2004, British Isles Lupus Assessment Group 2004; PtGA, Patient's Global Assessment; SLEDAI-2K, Systemic Lupus Erythematosus Disease Activity Index 2000; VAS, visual analog scale. Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day. BICLA response is defined as reduction of all baseline BILAG-2004 A and B scores and no worsening in other organ systems, no worsening from baseline in SLEDAI-2K, and no increase ≥0.30 points on a 3-point PtGA VAS from baseline.

FIG. 8: Combined BICLA and SIR (4) response and stringent BICLA response definitions at Week 52 in patients with SLE in the MUSE, TULIP-1, and TULIP-2 trials

Rates, differences, 95% Cis and nominal P values were calculated using a stratified Cochran-Mantel-Haenszel approach (stratification factors SLEDAI-2K score at screening, Day 1 GC dose, and IFNGS test status at screening). Response for all endpoints required no trial treatment discontinuation and no use of protocol-restricted medications. BICLA response, v baseline: improvements in all BILAG-2004 organ domains (A and B scores to B/C/D and C/D respectively, no BILAG-2004 domain worsening, SRI (4) response worsening; no PGA worsening (≥0.3 points).

FIG. 9: crBICLA response (requiring complete resolution of all BILAG-2004 A/B scores) in patients with SLE

crBICLA response criteria are defined in Table 9-1. Error bars represent standard error of the mean. * Nominal P<0.05; ** nominal P<0.01; *** nominal P<0.001.

FIG. 10: Delivery device

Anifrolumab is administered by an injection device [1] [9] such as a prefilled syringe (PFS) (FIG. 10A) or an autoinjector (AI) (FIG. 10B).

FIG. 11. Autoinjector

The autoinjector for administering anifrolumab of the functional variant thereof in exploded view (FIG. 11A), assembled (FIG. 11B) and filled with drug substance (FIG. 11C)

FIG. 12. Accessorized pre-filled syringe

The accessorized pre-filled syringe (APFS) for anifrolumab of the functional variant thereof. The primary tube is shown in assembled form (FIG. 12A) and in exploded view (FIG. 12B). The APFS with its additional components is shown in assembled form (FIG. 12C) and in exploded view FIG. 12D).

FIG. 13. Packaging for the delivery device

### 4 DETAILED DESCRIPTION

### 4.1 Method of Steroid Tapering

The invention relates to a method for steroid-sparing in a subject in need thereof, comprising administering a therapeutically effective amount of a type I IFN receptor (IFNAR1) inhibitor to the subject and a steroid, wherein the dose of the steroid administered to the subject is tapered from a pre-sparing dose at baseline to a post-sparing dose, wherein the subject has systemic lupus erythematosus (SLE).

The method may not worsen SLE disease activity in the subject. The post-sparing dose may be ≤75% of the pre-sparing dose. The post-sparing dose may be ≤50% of the pre-sparing dose. The post-sparing dose may be ≤25% of the pre-sparing dose. The post-sparing dose may be ≤10% of the pre-sparing dose. The post-sparing dose may be about 60% of the pre-sparing dose.

The pre-sparing steroid dose and post-sparing steroid dose may be daily doses. The pre-sparing steroid dose may be about ≥10 mg/day prednisone or prednisone-equivalent dose. The post-sparing steroid dose may be about ≤7 mg/day prednisone or prednisone-equivalent dose. The post-sparing steroid dose may be about ≤5 mg/day prednisone or prednisone-equivalent dose. The post-sparing dose may be maintained for ≥12 weeks. The post-sparing dose may be maintained for ≥12 weeks, where the post-sparing dose is ≤7.5 mg/day prednisone or prednisone-equivalent dose. The post-sparing dose may be maintained for ≥12 week, where the post-sparing dose is ≤5 mg/day prednisone or prednisone-equivalent dose. The post-sparing dose may be sustained for at least 1 week.

The invention also relates to a method for treating SLE in a subject in need thereof, comprising administering a therapeutically effective amount of a IFNAR1 inhibitor to the subject, wherein treatment reduces or prevents the need for increased administration of a steroid to the subject.

The method may have been demonstrated in a phase III clinical trial.

### 4.2 Preventing Organ Damage

The method of the invention may not worsen SLE disease activity in the subject. The method may reduce and/or prevent steroid associated side effects in the subject. The method may decreases the subject's blood pressure. The method may reduce and/or prevent steroid associated organ damage. The method may decrease the subject's diastolic blood pressure. The method may decrease the subject's systolic blood pressure. The method may decrease the subject's resting heart rate. The method may prevent an increase in the subject's blood pressure. The method may prevent an increase in the subject's diastolic blood pressure. The method may prevent an increase in the subject's systolic blood pressure.

### 4.3 Steroids

The steroid may be a glucocorticoid (GC). The steroid may comprises an oral glucocorticoid. The method of any preceding claims, wherein the steroid comprises hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof.

The steroid may comprise prednisone.

### 4.4 Reducing SLE Disease Activity

The method may reduce SLE disease activity in the subject. The reduction in SLE disease activity may comprise an improvement in the subject's SF-36 MCS score. The reduction in SLE disease activity may comprises a BICLA response. The reduction in SLE disease activity may comprise both a BICLA and SRI (4) response. The reduction in SLE disease activity may comprise a BICLA response, wherein the post-sparing dose is maintained for ≥12 weeks. The reduction in SLE disease activity may comprise a complete BICLA (crBICLA) response. The crBICLA response may be achieved by week 32 of treatment. The reduction in SLE disease activity may comprise a reduction in SLE flares. The method may increase the subject's body mass index (BMI). The method may increase the subject's weight. The subject may be underweight pre-treatment, wherein underweight is defined by BMI.

The ability of the IFNAR1 inhibitor to reduce SLE disease activity in a subject may have been demonstrated in a phase III clinical trial.

The method of any preceding claim, wherein the subject has moderate to severe SLE.

### 4.5 IFNAR1 Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-α/β receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist. The type I interferon receptor inhibitor may be an antibody or antigen-binding fragment thereof that inhibits type I IFN activity. Additionally or alternatively, the type I interferon receptor inhibitor may be a small molecule inhibitor of a type I interferon receptor (e.g. for pharmacological inhibition of type I interferon receptor activity).

The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain variable region complementarity determining region 2 (HCDR2) comprising the amino acid sequence of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of SEQ ID NO: 11; and (b) a human light chain comprising the amino acid sequence of SEQ ID NO: 12.

The IFNAR1 inhibitor may be anifrolumab or a functional variant thereof.

### 4.6 Doses and Methods of Administration

The method may comprise administering an intravenous dose of anifrolumab or the functional variant thereof to the subject. The intravenous dose may be ≥300 mg anifrolumab or the functional variant thereof. The intravenous dose may be ≤1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The subject may have moderate to severe SLE pre-treatment. The subject may have mild SLE. Moderate to severe SLE may be defined as a CLASI score of ≥10.

The subject may be a type I interferon stimulated gene signature (IFNGS)-test high patient pre-treatment. The method may comprise identifying the subject as IFNGS-test high patient pre-treatment.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

The method may comprise steroid sparing in the subject, wherein the dose of the steroid administered to the subject is tapered from a pre-sparing dose at baseline to a post-sparing dose. The post-sparing dose may be ≤7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

The invention also relates to a unit dose for use in the methods of the invention, wherein the unit dose comprises >105 mg and ≤150 mg anifrolumab or a functional variant thereof.

The unit dose may comprise ≤135 mg (i.e. 135 mg or less) anifrolumab or the functional variant thereof. The unit dose may comprise about 120 mg anifrolumab or the functional variant thereof. The unit dose may comprise 120 mg anifrolumab or the functional variant thereof. The unit dose may consist essentially of >105 mg and <150 mg anifrolumab or the functional variant thereof. The unit dose may consist essentially of ≤135 mg anifrolumab or the functional variant thereof. The unit dose may consist essentially of about 120 mg anifrolumab or the or the functional variant thereof. The concentration of anifrolumab or the functional variant thereof in the unit dose may be about 150 mg/ml. The volume of the unit dose may be less than 1 ml. The dose or unit dose may have a volume of about 0.5 to about 1 ml. The concentration of the unit dose may be about 0.8 ml. The volume of the unit dose may be 0.8 ml. The unit dose may comprise a formulation of about 150 to 200 mg/ml anifrolumab or the functional variant thereof, about 25 to 150 mM of lysine sale and an uncharged excipient. The unit dose may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

In another aspect the invention relates to a method of treating SLE in a subject, the method comprising subcutaneously administering a dose of anifrolumab or a functional variant thereof, wherein administering the dose every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is more than the plasma concentration provided by intravenous administration of 300 mg of anifrolumab or the functional variant thereof every 4 weeks. Administering the dose every week may provide a plasma concentration in the subject that is at least equivalent to the plasma concentration provided by intravenous administration of 400 mg of anifrolumab or the functional variant thereof every 4 weeks. The dose may be administered in a single-administration step. The dose administered to the subject may be <150 mg (i.e. less than 150 mg) anifrolumab or the functional variant thereof. The dose administered to the subject may be >105 mg (i.e. more than 105 mg) anifrolumab or the functional variant thereof. The dose of administered to the subject may be ≤135 mg (i.e. 135 mg or less) anifrolumab or the functional variant thereof. The dose administered to the subject may be about 120 mg anifrolumab or the functional variant thereof.

Administration of the dose or unit dose may provide a plasma concentration of anifrolumab or the functional variant thereof in the patient of >10 μg (i.e. 10 μg or more) anifrolumab or the functional variant thereof per ml of plasma (i.e. a plasma concentration of ≥10 μg/ml). Administration of the dose or unit dose may provide a plasma concentration of anifrolumab or the functional variant thereof in the subject of about 10-100 μg/ml. Administration of the dose or unit dose may provide a plasma concentration of anifrolumab or the functional variant thereof in the subject of about 20-80 μg/ml. Administration of the dose or unit dose may provide a plasma concentration of anifrolumab or the functional variant thereof in the subject of about 30-70 μg/ml. Administration of the dose or unit dose may provide a trough concentration of anifrolumab or the functional variant thereof in the subject of ≥ 20 μg/ml (i.e. 20 μg/ml or more). Administration of the dose or unit dose may provide a trough concentration of anifrolumab or the functional variant thereof in the subject of ≥30 μg/ml (i.e. 30 μg/ml or more). Administration of the dose or unit dose may provide a trough concentration of anifrolumab or the functional variant thereof in the subject of ≥40 μg/ml (i.e. 40 μg/ml or more). Administration of the dose or unit dose may provide a trough concentration of anifrolumab or the functional variant thereof in the subject of about 20-100 μg/ml. Administration of the dose or unit dose may provide a trough concentration of anifrolumab or the functional variant thereof in the subject of about 30-80 μg/ml. Administration of the dose or unit dose may provide a trough concentration of anifrolumab or the functional variant thereof in the subject of about 40-70 μg/ml.

### 4.7 the Subject

The subject may be a human subject. The subject may be an adult. The subject may be a patient with an elevated type I IFN gene signature. The subject may be a type I interferon stimulated gene signature (IFNGS)-test high patient pre-administration with the dose or unit dose. The subject may have elevated of the genes IFI27, IFI44, IFI44L, and RSAD2 in the whole blood. The method may comprise identifying the subject as IFNGS-test high patient pre-treatment with the dose or unit dose. The method may comprise measuring the expression of the genes IFI27, IFI44, IFI44L, and RSAD2 in the whole blood of the subject. The method may comprise measuring the expression of the genes IFI27, IFI44, IFI44L, and RSAD2 in the whole blood of the subject by RT-PCR.

The subject may have moderate to severe SLE.

### 4.8 Doses and Methods of Administration

The method may comprise administering an intravenous dose of anifrolumab or the functional variant thereof to the subject. The intravenous dose may be ≥300 mg anifrolumab or the functional variant thereof. The intravenous dose may be ≤1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The subject may have moderate to severe SLE pre-treatment. The subject may have mild SLE.

The subject may be a type I interferon stimulated gene signature (IFNGS)-test high patient pre-treatment. The method may comprise identifying the subject as IFNGS-test high patient pre-treatment.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

The method may comprise steroid sparing in the subject, wherein the dose of the steroid administered to the subject is tapered from a pre-sparing dose at baseline to a post-sparing dose. The post-sparing dose may be ≤7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

The invention also relates to a unit dose for use in the methods of the invention, wherein the unit dose comprises >105 mg and ≤150 mg anifrolumab or a functional variant thereof.

The unit dose may comprise ≤135 mg (i.e. 135 mg or less) anifrolumab or the functional variant thereof. The unit dose may comprise about 120 mg anifrolumab or the functional variant thereof. The unit dose may comprise 120 mg anifrolumab or the functional variant thereof. The unit dose may consist essentially of >105 mg and <150 mg anifrolumab or the functional variant thereof. The unit dose may consist essentially of ≤135 mg anifrolumab or the functional variant thereof. The unit dose may consist essentially of about 120 mg anifrolumab or the or the functional variant thereof. The concentration of anifrolumab or the functional variant thereof in the unit dose may be about 150 mg/ml. The volume of the unit dose may be less than 1 ml. The dose or unit dose may have a volume of about 0.5 to about 1 ml. The concentration of the unit dose may be about 0.8 ml. The volume of the unit dose may be 0.8 ml. The unit dose may comprise a formulation of about 150 to 200 mg/ml anifrolumab or the functional variant thereof, about 25 to 150 mM of lysine sale and an uncharged excipient. The unit dose may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

In another aspect the invention relates to a method of steroid sparing in a subject suffering from SLE, the method comprising subcutaneously administering a dose of anifrolumab or a functional variant thereof, wherein administering the dose every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is more than the plasma concentration provided by intravenous administration of 300 mg of anifrolumab or the functional variant thereof every 4 weeks. Administering the dose every week may provide a plasma concentration in the subject that is at least equivalent to the plasma concentration provided by intravenous administration of 400 mg of anifrolumab or the functional variant thereof every 4 weeks. The dose may be administered in a single-administration step. The dose administered to the subject may be <150 mg (i.e. less than 150 mg) anifrolumab or the functional variant thereof. The dose administered to the subject may be >105 mg (i.e. more than 105 mg) anifrolumab or the functional variant thereof. The dose of administered to the subject may be ≤135 mg (i.e. 135 mg or less) anifrolumab or the functional variant thereof. The dose administered to the subject may be about 120 mg anifrolumab or the functional variant thereof.

Administration of the dose or unit dose may provide a plasma concentration of anifrolumab or the functional variant thereof in the patient of ≥10 μg (i.e. 10 μg or more) anifrolumab or the functional variant thereof per ml of plasma (i.e. a plasma concentration of ≥10 μg/ml). Administration of the dose or unit dose may provide a plasma concentration of anifrolumab or the functional variant thereof in the subject of about 10-100 μg/ml. Administration of the dose or unit dose may provide a plasma concentration of anifrolumab or the functional variant thereof in the subject of about 20-80 μg/ml. Administration of the dose or unit dose may provide a plasma concentration of anifrolumab or the functional variant thereof in the subject of about 30-70 μg/ml. Administration of the dose or unit dose may provide a trough concentration of anifrolumab or the functional variant thereof in the subject of ≥ 20 μg/ml (i.e. 20 μg/ml or more). Administration of the dose or unit dose may provide a trough concentration of anifrolumab or the functional variant thereof in the subject of ≥30 μg/ml (i.e. 30 μg/ml or more). Administration of the dose or unit dose may provide a trough concentration of anifrolumab or the functional variant thereof in the subject of ≥40 μg/ml (i.e. 40 μg/ml or more). Administration of the dose or unit dose may provide a trough concentration of anifrolumab or the functional variant thereof in the subject of about 20-100 g/ml. Administration of the dose or unit dose may provide a trough concentration of anifrolumab or the functional variant thereof in the subject of about 30-80 μg/ml. Administration of the dose or unit dose may provide a trough concentration of anifrolumab or the functional variant thereof in the subject of about 40-70 μg/ml.

The dose or unit dose may provide a therapeutic effect in the subject that is at least equivalent to a therapeutic effect provided by administration of an intravenous dose of 300 mg anifrolumab or the functional variant thereof administered once every (Q4W). The dose or unit dose may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The methods of the invention may comprise administering the dose or unit dose at intervals of 6-8 days. The dose or unit dose may be administered once per week (QW). The dose or unit dose may be 120 mg anifrolumab or the functional variant thereof, wherein the method comprises administering the dose in a single administration step once per week (QW). In other words, the method comprises administering 120 mg QW of anifrolumab of the functional variant thereof. The dose or unit dose may be administered once per week for at least about 4 weeks. The dose or unit dose may be administered once per week for at least about 8 weeks. The dose or unit dose may be administered once per week for at least about 12 weeks. The dose or unit dose may be administered once per week for at least about 16 weeks. The dose or unit dose may be administered once per week for at least about 20 weeks. The dose or unit dose may be administered once per week for at least about 24 weeks. The dose or unit dose may be administered once per week for at least about 28 weeks. The dose or unit dose may be administered once per week for at least about 32 weeks. The dose or unit dose may be administered once per week for about 8 weeks. The dose or unit dose may have a volume permitted it suitable delivery in a single subcutaneous administration step. The dose or unit dose may have a volume of about 0.5 to about 1 ml. The dose or unit dose may have a volume of less than 1 ml. The dose or unit dose may have a volume of about 0.8 ml.

### 4.9 Pharmaceutical Composition

The invention also relates to a pharmaceutical composition for use in a method of treating SLE in a subject, the method comprising subcutaneously administering the pharmaceutical composition to a subject, wherein the pharmaceutical composition comprises a dose of anifrolumab or functional variant thereof, wherein the dose is >105 mg and <150 mg. The dose of anifrolumab of the functional variant thereof may be a unit dose (unit dose form, pharmaceutical unit dose form, pharmaceutical unit dose). Functional anifrolumab variants include antigen-binding fragments of anifrolumab and antibody and immunoglobulin derivatives of anifrolumab.

In another aspect the invention relates to a pharmaceutical composition for use in the method of the invention, the method comprising subcutaneously administering the pharmaceutical composition to the subject, wherein the pharmaceutical composition comprises a dose of anifrolumab or functional variant thereof, wherein administering the pharmaceutical composition every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is about equivalent to the plasma concentration provided by intravenous administration of 400 mg of anifrolumab or the functional variant thereof every 4 weeks. The dose may be <150 mg (i.e. less than 150 mg) anifrolumab or the functional variant thereof. The dose may be >105 mg (i.e. more than 105 mg) anifrolumab or the functional variant thereof. The dose may be ≤135 mg (i.e. 135 mg or less) anifrolumab or the functional variant thereof. The dose may be about 120 mg anifrolumab or the functional variant thereof. The dose may be 120 mg anifrolumab or the functional variant thereof.

The pharmaceutical composition may be administered at intervals of 6-8 days. The pharmaceutical composition may be administered once per week (QW). The pharmaceutical composition may be administered in a single administration step. The dose may be 120 mg anifrolumab or the functional variant thereof, and the method of treatment may comprise administering the dose in a single administration step once per week (QW). The pharmaceutical composition may be administered once per week for at least about 4 weeks. The pharmaceutical composition may be administered once per week for at least about 8 weeks. The dose or unit dose may be administered once per week for at least about 12 weeks. The pharmaceutical composition may be administered once per week for at least about 16 weeks. The pharmaceutical composition may be administered once per week for at least about 20 weeks. The pharmaceutical composition may be administered once per week for at least about 24 weeks. The pharmaceutical composition may be administered once per week for at least about 28 weeks. The pharmaceutical composition may be administered once per week for at least about 32 weeks. The pharmaceutical composition may be administered once per week for about 8 weeks. The pharmaceutical composition may have a volume permitted it suitable delivery in a single subcutaneous administration step. The pharmaceutical composition may have a volume of about 0.5 to about 1 ml. The pharmaceutical composition may have a volume of less than 1 ml. The pharmaceutical composition may have a volume of about 0.8 ml.

Administration of the pharmaceutical composition may provide a plasma concentration of anifrolumab or the functional variant thereof in the patient of ≥10 μg (i.e. 10 μg or more) anifrolumab or the functional variant thereof per ml of plasma (i.e. a plasma concentration of ≥10 μg/ml). Administration of the pharmaceutical composition may provide a plasma concentration of anifrolumab or the functional variant thereof in the subject of about 10-100 μg/ml. Administration of the pharmaceutical composition may provide a plasma concentration of anifrolumab or the functional variant thereof in the subject of about 20-80 μg/ml. Administration of the pharmaceutical composition may provide a plasma concentration of anifrolumab or the functional variant thereof in the subject of about 30-70 μg/ml. Administration of the pharmaceutical composition may provide a trough concentration of anifrolumab or the functional variant thereof in the subject of >20 μg/ml (i.e. 20 μg/ml or more). Administration of the pharmaceutical composition may provide a trough concentration of anifrolumab or the functional variant thereof in the subject of ≥30 μg/ml (i.e. 30 μg/ml or more). Administration of the pharmaceutical composition may provide a trough concentration of anifrolumab or the functional variant thereof in the subject of ≥40 μg/ml (i.e. 40 μg/ml or more). Administration of the pharmaceutical composition may provide a trough concentration of anifrolumab or the functional variant thereof in the subject of about 20-100 μg/ml. Administration of the pharmaceutical composition may provide a trough concentration of anifrolumab or the functional variant thereof in the subject of about 30-80 μg/ml. Administration of the pharmaceutical composition may provide a trough concentration of anifrolumab or the functional variant thereof in the subject of about 40-70 μg/ml.

The pharmaceutical composition may provide a therapeutic effect in the subject that is at least equivalent to a therapeutic effect provided by administration of an intravenous dose of 300 mg anifrolumab or the functional variant thereof administered once every (Q4W). The pharmaceutical composition may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

### 4.10 Device

The invention also relates to an injection device comprising the unit dose of the invention, or the pharmaceutical composition for the use of any of the invention.

The pharmaceutical in the injection device may comprise >105 mg (i.e. more than 105 mg) and <150 mg (i.e. less than 150 mg) anifrolumab or a functional variant thereof. The pharmaceutical composition in the injection device may comprise about 120 mg anifrolumab or the functional variant thereof. The pharmaceutical composition in the injection device may comprise 120 mg anifrolumab or the functional variant thereof. The concentration of anifrolumab or the functional variant thereof in the pharmaceutical composition in the injection device may be 150 mg/ml. The volume of the pharmaceutical composition in the injection device may be at least about 0.8 ml. The volume of the pharmaceutical composition may be about 0.8 ml.

The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/ml anifrolumab or the functional variant thereof. The pharmaceutical composition in the injection device may comprise 50 mM lysine HCl. The pharmaceutical composition may comprise 130 mM trehalose dihydrate. The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition may comprise 50 mM lysine HCl. The pharmaceutical composition in the injection device may comprise 130 mM trehalose dihydrate. The pharmaceutical composition in the injection device may comprise 0.05% polysorbate 80. The pharmaceutical composition in the injection device may comprise 25 mM histidine/histidine HCl. The pharmaceutical composition in the injection device may comprise 150 mg/mL anifrolumab or the functional variant thereof, 50 mM lysine HCl, 130 mM trehalose dihydrate, 0.05% polysorbate 80 and 25 mM histidine/histidine HCl.

In another aspect the invention relates to an injection device comprising a unit dose. The unit dose may comprise >105 mg (i.e. at least 105 mg) and <150 mg (i.e. less than 150 mg) anifrolumab or a functional variant thereof. The unit dose may comprise ≤135 mg (i.e. 135 mg or less) anifrolumab or the functional variant thereof. The unit dose may comprise about 120 mg anifrolumab or the functional variant thereof. The unit dose in the injection device may comprise 120 mg anifrolumab or the functional variant thereof. The unit dose in the injection device may consist essentially of >105 mg and <150 mg anifrolumab or the functional variant thereof. The unit dose in the injection device may consist essentially of ≤135 mg anifrolumab or the functional variant thereof. The unit dose in the injection device may consist essentially of about 120 mg anifrolumab or the or the functional variant thereof. The concentration of anifrolumab or the functional variant thereof in the unit dose in the injection device may be about 150 mg/ml. The volume of the unit dose in the injection device may be less than 1 ml. The unit dose in the injection device may have a volume of about 0.5 to about 1 ml. The concentration of the unit dose may be about 0.8 ml. The volume of the unit dose may be 0.8 ml. The unit dose in the injection device may comprise a formulation of about 150 to 200 mg/ml anifrolumab or the functional variant thereof, about 25 to 150 mM of lysine sale and an uncharged excipient. The unit dose in the injection device may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

The injection device may be a pre-filled syringe (PFS). The injection device may be an accessorized pre-filed syringe (AFPS). The injection device may be an auto-injector (AI).

### 4.11 Kit

In another aspect the invention relates to a kit comprising a unit dose of the invention and instructions for use, wherein the instructions for use comprise instructions for subcutaneous administration of the unit dose to a subject. In another aspect the invention relates to a kit comprising the pharmaceutical composition for the use of the invention, wherein the instructions for use comprise instructions for subcutaneous administration of the pharmaceutical composition to a subject. In another aspect the invention relates to a kit comprising the injection device of any of the invention, and instructions for use, wherein the instruction for use comprise instructions for use of the injection device to subcutaneously administer the unit dose or pharmaceutical composition to the subject.

The kit of the invention may comprise packaging, wherein the packaging is adapted to hold the injection device and the instructions for use. The instructions for use may be attached to the injection device. The instruction for use may comprise instructions for administration of >105 mg and <150 mg anifrolumab or functional variant thereof. The instruction for use may comprise instructions for administration of ≤135 mg anifrolumab or the functional variant thereof. The instruction for use may comprise instructions for administration of 120 mg anifrolumab or the functional variant thereof. The instruction for use may comprise instructions for administration of 120 mg anifrolumab or the functional variant thereof every 4 weeks. The instructions for use may define the subject as having a type I IFN mediated disease. The instructions may define the subject as having SLE. The instructions may define the subject as having moderate to severe SLE. The instructions for use may be written instructions.

The instructions for use may specify that the injection device, unit dose and/or pharmaceutical composition are for use in the treatment of SLE. The instructions for use comprise instructions for administration of 120 mg anifrolumab or the functional variant thereof every week.

The instructions for use may specify that administration of the IFNAR1 inhibitor to a subject permits steroid tapering. The instructions for use may specify that the subject has moderate to severe SLE. The instructions for use may specify that the subject has active SLE. The instructions for use may specify that the subject has steroid associated organ damage. The instructions for use may specify tapering the steroid dose administered to the subject.

The instructions for use may specify that administration of the IFNAR1 inhibitor to a subject may permit steroid tapering from a pre-sparing steroid dose at baseline to a post-sparing steroid dose. The post-sparing dose may be ≤75%, ≤50%, ≤25% or ≤10% of the pre-sparing dose. The instructions for use may specify that the steroid comprises hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof.

The instructions for use may specify that administration of the IFNAR1 inhibitor to a subject may reduce and/or prevents steroid associated side effects in the subject. The instructions for use may specify that administration of the IFNAR1 inhibitor to a subject may reduce SLE disease activity in the subject. The reduction in SLE disease activity may comprise an improvement in the subject's SF-36 MCS score. The instruction for use may specify that the reduction in SLE disease activity may comprise a BICLA response. The instruction for use may specify that the reduction in SLE disease activity may comprise both a BICLA and SRI (4) response. The instruction for use may specify that the reduction in SLE disease activity may comprise a BICLA response, wherein the instructions for use specify that the post-sparing dose should be maintained for ≥12 weeks. The instruction for use may specify that the reduction in SLE disease activity comprises a complete BICLA (crBICLA) response. The instruction for use may specify that the crBICLA response may be achieved by week 32 of treatment. The instruction for use may specify that the reduction in SLE disease activity may comprise a reduction in SLE flares.

The instruction for use may specify that the administration of the IFNAR1 inhibitor may increase the subject's body mass index (BMI). The instruction for use may specify that the administration of the IFNAR1 may increase the subject's weight.

The instructions for use may specify that the ability of the IFNAR1 inhibitor to reduce SLE disease activity in a subject has been demonstrated in a phase III clinical trial.

The instructions for use may specify that the IFNAR1 inhibitor is anifrolumab or a functional variant thereof.

The instructions for use may specify a method comprising administering to the subject a therapeutically effective amount of a type I IFN receptor (IFNAR1) inhibitor and a steroid, wherein the dose of the steroid administered to the subject is tapered from a pre-sparing dose at baseline to a post-sparing dose, wherein the subject has systemic lupus erythematosus (SLE). The instructions may specify that the method does not worsen SLE disease activity. The instructions may specify that the post-sparing dose is ≤75% of the pre-sparing dose. The instructions may specify that post-sparing dose is ≤50% of the pre-sparing dose. The instructions may specify that the post-sparing dose is ≤25% of the pre-sparing dose. The instructions may specify that the post-sparing dose is ≤10% of the pre-sparing dose. The instructions may specify that the post-sparing dose is about 60% of the pre-sparing dose, wherein the pre-sparing steroid dose and post-sparing steroid dose are daily doses.

The instructions for use may specify performing any of the methods of the invention.

The instructions may specify that the pre-sparing steroid dose is about ≥10 mg/day prednisone or prednisone-equivalent dose. The instructions may specify that the post-sparing steroid dose is about ≤7 mg/day prednisone or prednisone-equivalent dose. The instructions may specify that the post-sparing steroid dose is about ≤5 mg/day prednisone or prednisone-equivalent dose. The instructions may specify that the post-sparing dose should be maintained for ≥12 weeks. The instructions may specify that the post-sparing dose should be maintained for ≥12 weeks and the post-sparing dose should be ≤7.5 mg/day prednisone or prednisone-equivalent dose. The instructions may specify that the post-sparing dose should be maintained for >12 weeks and the post-sparing dose should be ≤5 mg/day prednisone or prednisone-equivalent dose. The instructions may specify that the post-sparing dose may be about 0 mg/day. prednisone or prednisone-equivalent dose. The instructions may specify that the post-sparing dose should be sustained for at least 1 week.

### 4.12 Formulations

The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

Stable formulations suitable for administration to subjects and comprising anifrolumab are described in detail in U.S. Pat. No. 10,125,195 B1, which is incorporated herein in its in entirety.

### 5 Definitions

### 5.1 Type I IFN Receptor Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-α/β receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist.

Additionally or alternatively, the type I interferon receptor inhibitor may be a small molecule inhibitor of a type I interferon receptor (e.g. for pharmacological inhibition of type I interferon receptor activity).

The type I interferon receptor inhibitor may be an antibody or antigen-binding fragment thereof that inhibits type I IFN activity. A particularly preferred type I interferon receptor inhibitor is the antibody anifrolumab or a functional variant thereof. Anifrolumab is a monoclonal antibody targeting IFNAR1 (the receptor for a, B, and w interferons). Disclosure related to anifrolumab can be found in U.S. Pat. Nos. 7,662,381 and 9,988,459, which are incorporated herein by reference.

### 5.1.1 Anifrolumab

Anifrolumab (MEDI-546, anifro, ANI) is a human immunoglobulin G1 kappa (IgG1_{K}) monoclonal antibody (mAb) directed against subunit 1 of the type I interferon receptor (IFNAR1). Anifrolumab downregulates IFNAR signaling and suppresses expression of IFN-inducible genes. Disclosures related to anifrolumab can be found in U.S. Pat. Nos. 7,662,381 and 9,988,459, which are incorporated herein by reference in their entirety. Sequence information for anifrolumab is provided in Table 5-1: Sequences.

TABLE5-1
Sequences

| | |
|---|---|
| Anifrolumab VH (SEQ ID NO: 1) | EVQLVQSGAEVKKPGESLKISCKGSGYIFTNYWIAWVRQMPGKGLESMGllYPGD |
| | SDIRYSPSFQGQVTISADKSITTAYLQWSSLKASDTAMYYCARHDIEGFDYWGRG |
| | TLVTVSS |
| Anifrolumab VL (SEQ ID NO: 2) | EIVLTQSPGTLSLSPGERATLSCRASQSVSSSFFAWYQQKPGQAPRLLIYGASSR |
| | ATGIPDRLSGSGSGTDFTLTITRLEPEDFAVYYCQQYDSSAITFGQGTRLEIK |
| HCDR1 (SEQ ID NO: 3) | NYWIA |
| HCDR2 (SEQ ID NO: 4) | IIYPGDSDIRYSPSFQG |
| HCDR3 (SEQ ID NO: 5) | HDIEGFDY |
| LCDR1 (SEQ ID NO: 6) | RASQSVSSSFFA |
| LCDR2 (SEQ ID NO: 7) | GASSRAT |
| LCDR3 (SEQ ID NO: 8) | QQYDSSAIT |
| Light chain constant region | RTVAAPSVFIFPPSDEQLKSGTASVVCLLNNFYPREAKVQWKVDNALQSGNSQES |
| (SEQ ID NO: 9) | VTEQDSKDSTYSLSSTLTLSKADYEKHKVYACEVTHQGLSSPVTKSFNRGEC |
| Heavy chain constant region | ASTKGPSVFPLAPSSKSTSGGTAALGCLVKDYFPEPVTVSWNSGALTSGVHTFPA |
| (SEQ ID NO: 10) | VLQSSGLYSLSSVVTVPSSSLGTQTYICNVNHKPSNTKVDKRVEPKSCDKTHTCP |
| | PCPAPEFEGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPASIEKTIS |
| | KAKGQPREPQVYTLPPSREEMTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| Heavy chain | EVQLVQSGAEVKKPGESLKISCKGSGYIFTNYWIAWVRQMPGKGLESMGIIYPGD |
| (SEQ ID NO: 11) | SDIRYSPSFQGQVTISADKSITTAYLOWSSLKASDTAMYYCARHDIEGFDYWGRG |
| | TLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKRVEPKSC |
| | DKTHTCPPCPAPEFEGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | SIEKTISKAKGQPREPQVYTLPPSREEMTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| Light chain | EIVLTQSPGTLSLSPGERATLSCRASQSVS SSFFAWYQQK PGQAPRLLIY |
| (SEQ ID NO: 12) | GASSRATGIPDRLSGSGSGT DFTLTITRLE PEDFAVYYCQ QYDSSAITFG |
| | QGTRLEIKRTVAAPSVFIFPPSDEQLKSGT ASVVCLLNNF YPREAKVQWK |
| | VDNALQSGNSQESVTEQDSKDSTYSLSSTLTLSKADYEKHKVYACEVTHQGLSSP |
| | VTKSFNRGEC |

Anifrolumab is an immunoglobulin comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). Anifrolumab is an immunoglobulin comprising a VH of SEQ ID NO: 1 and a VL of SEQ ID NO: 2.

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

For example, a variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

A variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and preferably with the same affinity.

Functional variants of anifrolumab include the antibodies described in WO 2018/023976 A1, incorporated herein by reference (Table 5-2).

TABLE5-2
anti-IFNAR antibody sequences

| Description | SEQ ID | Sequence |
|---|---|---|
| H15D10 (VH) | 13 | EVQLVQSGAEVKKPGESLRISCKGSGYTFTNYWVAWVRQMPGKGLESMG |
| | | IIYPGDSDTRYSPSFQGHVTISADKSISTAY |
| L8C3 (VL) | 14 | DIQMTQSPSSLSASLGDRVTITCRASQNVGNYLNWYQQKPGKAPKLLIY |
| | | RASNLASGVPSRFSGSGSGTDFTLTISSLQPEDFATYYCQQMEHAPPTF |
| | | GQGTKVEIKR |
| L16C11 (VL) | 15 | EIVLTQSPGTLSLSPGERATLSCRASQSVIGYYLAWYQQKPGQAPRLLI |
| | | YSVSTLASGIPDRFSGSGSGTDFTLTISRLEPEDFAVYYCQQYYRFPIT |
| | | FGQGTKVEIK |
| H19B7 (VH) | 16 | EVQLVQSGAEVKKPGESLRISCKGSGYTFTNYWMAWVRQMPGKGLESMG |
| | | IIYPSDSDTRYSPSFQGHVTISADKSISTAYLQWSSLKASDTAMYYCAR |
| | | HDVEGYDYWGQGTLVTVSS |

Functional variants include antibodies comprising the VH amino acid sequence SEQ ID NO: 13. Functional variants include antibodies comprising the VH amino acid sequence SEQ ID NO: 16. Functional variants include antibodies comprising the VL amino acid sequence SEQ ID NO: 14. Functional variants include antibodies comprising the VL amino acid sequence SEQ ID NO: 15. Functional variants include antibodies comprising the VH amino acid sequence SEQ ID NO: 16. Functional variants include antibodies comprising the VH sequence SEQ ID NO: 13 and VL amino acid sequence SEQ ID NO: 16. Functional variants include antibodies comprising the VH sequence SEQ ID NO: 13 and VL amino acid sequence SEQ ID NO: 15. Functional variants include antibodies comprising the VH sequence SEQ ID NO: 16 and VL amino acid sequence SEQ ID NO: 15. Functional variants include antibodies comprising the VH sequence SEQ ID NO: 16 and VL amino acid sequence SEQ ID NO: 14.

IFNAR inhibitors may be a monoclonal antibody comprising the VH amino acid sequence SEQ ID NO: 13. The anti-IFNAR antibodies may comprise the VH amino acid sequence SEQ ID NO: 16. The anti-IFNAR antibodies may comprise the VL amino acid sequence SEQ ID NO: 14. The anti-IFNAR antibodies may comprise the VL amino acid sequence SEQ ID NO: 15. The anti-IFNAR antibodies may comprise the VL amino acid sequence SEQ ID NO: 16. The anti-IFNAR antibodies may comprise the VH sequence SEQ ID NO: 13 and VL amino acid sequence SEQ ID NO: 16. The anti-IFNAR antibodies may comprise the VH sequence SEQ ID NO: 13 and VL amino acid sequence SEQ ID NO: 15. The anti-IFNAR antibodies may comprise the VH sequence SEQ ID NO: 16 and VL amino acid sequence SEQ ID NO: 15. The anti-IFNAR antibodies may comprise the VH sequence SEQ ID NO: 16 and VL amino acid sequence SEQ ID NO: 14.

Functional variants of anifrolumab and anti-IFNAR antibodies include the QX006N antibody described in CN 11327807, incorporated herein by reference.

TABLE3
QX006N antibody sequences

| Description | SEQ ID NO | Sequence |
|---|---|---|
| QX006N (VH) | 17 | EVQLVESGGGLVQPGGSLRLSCAASGFSLSSYYMTWVRQAPGKGLEW |
| | | VSVINVYGGTYYASWAKGRFTISRDNSKNTLYLQMNSLRAEDTAVYY |
| | | CAREDVAVYMAIDLWGQGTLVTVSS |
| QX006N (VL) | 18 | AIQMTQSPSSLSASVGDRVTITCQASQSISNQLSWYQQKPGKAPKLL |
| | | IYDASSLASGVPSRFSGSRSGTKFTLTISSLQPEDFATYYCLGIYGD |
| | | GADDGIAFGGGTKVEIK |
| QX006N (HCDR1) | 19 | SYYMT |
| QX006N (HCDR2) | 20 | VINVYGGTYYASWAKG |
| QX006N (HCDR3) | 21 | EDVAVYMAIDL |
| QX006N (LCDR1) | 22 | QASQSISNQLS |
| QX006N (LCDR2) | 23 | DASSLAS |
| QX006N (LCDR3) | 24 | LGIYGDGADDGIA |

IFNAR inhibitors may be a monoclonal antibody comprising the VH amino acid sequence SEQ ID NO: 17. The anti-IFNAR antibodies may comprise the VL amino acid sequence SEQ ID NO: 18.

QX006N is an immunoglobulin comprising an HCDR1, HCDR2 and HCDR3 of SEQ ID NO: 19, SEQ ID NO: 20, and SEQ ID NO: 21, respectively (or functional variant thereof); and an LCDR1, LCDR2 and LCDR3 of SEQ ID NO: 22, SEQ ID NO: 23, and SEQ ID NO: 23, respectively (or functional variant thereof). QX006N is an immunoglobulin comprising a VH amino acid sequence SEQ ID NO: 17 the VL amino acid sequence SEQ ID NO: 18.

### 5.2 Steroids

Oral corticosteroids (OCS, glucocorticoids) include prednisone, cortisone, hydrocortisone, methylprednisolone, prednisolone and triamcinolone. Examples of equivalent doses of oral prednisone are shown in Table 5-4.

TABLE 5-4
Examples of equivalent doses of oral prednisone

| Oral Prednisone and | | | | | |
|---|---|---|---|---|---|
| Equivalents | Equivalent Dose | | | | |
| Oral Prednisone | 7.5 mg | 10 mg | 20 mg | 30 mg | 40 mg |
| Cortisone | 37.5 mg | 50 mg | 100 mg | 150 mg | 200 mg |
| Hydrocortisone | 30 mg | 40 mg | 80 mg | 120 mg | 160 mg |
| Methylprednisolone | 6 mg | 8 mg | 16 mg | 24 mg | 32 mg |
| Prednisolone | 7.5 mg | 10 mg | 20 mg | 30 mg | 40 mg |
| Triamcinolone | 6 mg | 8 mg | 16 mg | 24 mg | 32 mg |

### 5.3 Clinical Trials

### 5.3.1 Phase 2/Phase II/Pivotal Studies

Phase II studies gather preliminary data on effectiveness. In Phase 2 studies, researchers administer the drug to a group of patients with the disease or condition for which the drug is being developed. Typically involving a few hundred patients, these studies aren't large enough to show whether the drug will be beneficial. Instead, Phase 2 studies provide researchers with additional safety data. Researchers use these data to refine research questions, develop research methods, and design new Phase 3 research protocols.

### 5.3.2 Phase 3/Phase III/Pivotal Studies or Trials

Researchers design Phase 3 studies to demonstrate whether or not a product offers a treatment benefit to a specific population. Sometimes known as pivotal studies, these studies involve 300 to 3,000 participants. Phase 3 studies provide most of the safety data. In previous studies, it is possible that less common side effects might have gone undetected. Because these studies are larger and longer in duration, the results are more likely to show long-term or rare side effects. Regulatory bodies such as the EMA and FDA usually require a phase III clinical trial demonstrating that the product is safe and at least as effective (if not better) than available medications, before approving a new medication. Phase III clinical trials usually fail, even if they follow a successful a phase II clinical trial.

### 5.4 Dosage Forms

A unit dose (also referred to as a unit dose form, a pharmaceutical unit dose or a pharmaceutical unit dose form) is a dose formed from a single unit. A unit dose (unit dose form) is suitable for administration to a subject in a single administration step. A unit dose (unit dose form) may be packaged in a single-unit container, for example a single-use pre-filled syringe or autoinjector. Unit doses provide the advantage that they can be ordered, packaged, handled and administered as single dose units containing a pre-determined amount of a drug. Unit doses decrease administration errors and reduce waste.

### 5.5 PK/PD

Plasma levels obtainable by SC administration and IV administration may be compared on the basis of a plasma drug concentration-time curve (AUC), which reflects the body exposure to the antibody after administration of a dose of the drug. For example, during a clinical study, the patient's plasma drug concentration-time profile can be plotted by measuring the plasma concentration at several time points. Where an in silico modelling approach is employed, plasma drug concentration-time for any given dose may be predicted. The AUC (area under the curve) can then be calculated by integration of the plasma drug concentration-time curve. Suitable methodology is described in Tummala et. al.⁴¹, which is incorporated herein by reference in its entirety. In the Examples described herein, PK parameters were calculated by non-compartmental analysis with Phoenix WinNonlin V/6.2 (Certara, Inc., Princeton, New Jersey, USA) and included the area under the serum concentration-time curve (AUC), clearance (CL, CL/F), maximum serum concentration (Cₘₐₓ) and time to reach maximum serum concentration (tₘₐₓ). All data were analysed with SAS System V.9.2 (SAS Institute, Inc., Cary, NC, USA).

Conveniently, a ratio of the AUC obtainable with SC administration to the AUC obtainable by IV administration (AUC_{SC}/AUC_{IV}) may be calculated, providing a numerical comparison of bioavailability provided by the dosage routes. Reference to the “AUC Ratio” herein means the AUC_{SC}/AUC_{IV} ratio. To provide statistical robustness, the AUC ratio is preferably a mean, median or mode (for example, a mean) value calculated from a plurality of repeat experiments (or computational simulations). This approach is demonstrated with reference to the Examples. The mean, median or mode (preferably mean) may be derived by pooling data obtained from multiple patients (or multiple computational simulations). Thus, the AUC Ratio may reflect the mean, median or mode (preferably mean) AUC in multiple patients.

### 5.6 Pharmacokinetics Glossary

Area under the curve (AUC): Area under the plasma drug concentration versus time curve, which serves as a measure of drug exposure.

Cₐᵥₑ: Steady-state average concentration.

Cₘₐₓ: The maximum (or peak) concentration of the drug in the plasma.

Cₘᵢₙ: Minimum plasma drug concentration.

C_{trough}: the concentration of drug in plasma at steady state immediately prior to the administration of a next dose. Trough plasma concentration (measured concentration at the end of a dosing interval at steady state [taken directly before next administration]).

LLOQ: The lower limit of quantitation, the lowest amount of an analyte in a sample that can be quantitatively determined with suitable precision and accuracy.

Linear pharmacokinetics: When the concentration of the drug in the blood or plasma increases proportionally with the increasing dose, and the rate of elimination is proportional to the concentration, the drug is said to exhibit linear pharmacokinetics. The clearance and volume of distribution of these drugs are dose-independent.

Nonlinear pharmacokinetics: As opposed to linear pharmacokinetics, the concentration of the drug in the blood or plasma does not increase proportionally with the increasing dose. The clearance and volume of distribution of these may vary depending on the administered dose. Nonlinearity may be associated with any component of the absorption, distribution, and/or elimination processes.

### 5.7 Delivery Device

As well as providing for subcutaneous administration of the antibody, the ability to self-administer (e.g. for home use) may further be enhanced by subcutaneous administration via an accessorized pre-filled syringe (APFS), an autoinjector (AI), or a combination thereof. Such devices have been found to be well-tolerated and reliable for administering subcutaneous doses of an antibody and provide further options for optimizing patient care. Indeed, such devices may reduce the burden of frequent clinic visits for patients. An example of a suitable APFS device is described in Ferguson et. al.⁴², which is incorporated herein by reference in its entirety.

The dose elucidated by the inventors provides yet advantages in the context of APFS-administration, as an APFS device typically administers a maximal volume of 1 ml. A dose in the range of >105 mg to <155 mg can be readily accommodated by a volume of ˜0.8 ml, such that the dose(s) of the present invention are uniquely suited to APFS and AI administration. For comparison, due to viscosity of the anifrolumab, larger doses (particularly doses of >150 mg) would need to be administered within a volume of >1 ml, requiring at least two SC injections, which is inconvenient for the patient, and would require a plurality of pre-filled devices.

The delivery device may be single use, disposable system that is designed to enable manual, SC administration of the dose.

### 5.8 End Points

### 5.8.1 BILAG-2004 (British Isles Lupus Assessment Group-2004)

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorises disease activity into 5 different levels from A to E:
Grade A represents very active disease requiring immunosuppressive drugs and/or a prednisone dose of >20 mg/day or equivalent
Grade B represents moderate disease activity requiring a lower dose of corticosteroids, topical steroids, topical immunosuppressives, antimalarials, or NSAIDs
Grade C indicates mild stable disease.
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

BILAG-defined improvement in mucocutaneous or musculoskeletal organ systems were representative of rash or arthritis, respectively.

### 5.8.2 BICLA (BILAG-Based Composite Lupus Assessment)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (≤10%) in physicians global assessment; and (5) no treatment failure (initiation of non-protocol treatment).

Particularly, a subject is a BICLA responder if the following criteria are met:
a) Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
b) No worsening from baseline in SLEDAI-2K as defined as an increase from baseline of >0 points in SLEDAI-2K;
c) No worsening from baseline in the subjects' lupus disease activity defined by an increase ≥0.30 points on a 3-point PGA VAS;
d) No discontinuation of investigational product or use of restricted medications beyond the protocol-allowed threshold before assessment

A complete resolution (crBICLA, also referred to a modified BICLA (mBICLA)) response requires a complete resolution of all baseline BILAG-2004 activity (all baseline A/B scores to D; no worsening of C or D scores).

### 5.8.3 CLASI (Cutaneous Lupus Erythematosus Disease Area and Severity Index Inflammatory Disease Activity)

The Cutaneous Lupus Erythematosus Disease Area and Severity Index (CLASI) was developed in 2005 as a means of specifically tracking cutaneous activity and damage in patients with CLE⁴³. The CLASI is a simple, single-page tool that separately quantifies skin disease activity and damage in each part of the body⁴⁴. The CLASI features a skin activity summary score (CLASI-A) and damage summary score (CLASI-D).

The Cutaneous Lupus Erythematosus Disease Area and Severity Index (CLASI) quantifies disease activity and damage in cutaneous lupus erythematosus. It can distinguish between different response levels of treatment, e.g., it is able to detect a specific percentage reduction in activity score from baseline, or can be reported by a mean/median score. Particularly, the CLASI is a validated index used for assessing the cutaneous lesions of lupus and consists of 2 separate scores: the first summarizes the inflammatory activity of the disease; the second is a measure of the damage done by the disease. The activity score takes into account erythema, scale/hypertrophy, mucous membrane lesions, recent hair loss, and nonscarring alopecia. The damage score represents dyspigmentation, scarring/atrophy/panniculitis, and scarring of the scalp. Subjects are asked if their dyspigmentation lasted 12 months or longer, in which case the dyspigmentation score is doubled. Each of the above parameters is measured in 13 different anatomical locations, included specifically because they are most often involved in cutaneous lupus erythematosus (CLE). The most severe lesion in each area is measured.

Modified CLASI (mCLASI) is defined as the activity portions of CLASI that describe skin erythema, scale/hypertrophy, and inflammation of the scalp. Activity of oral ulcers and alopecia without scalp inflammation are excluded from the mCLASI analysis, as are all measures of damage. Clinically meaningful improvement in rash, as measured using mCLASI, is defined by ≥50% decrease in baseline activity score.

### 5.8.4 SRI (Systemic Lupus Erythematosus Responder Index of ≥4)

A subject achieves SRI (4) if all of the following criteria are met:
Reduction from baseline of ≥4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase ≥0.30 points on a 3-point PGA VAS.

SRI(X) (X=5, 6, 7, or 8) is defined by the proportion of subjects who meet the following criteria:
Reduction from baseline of ≥X points in the SLEDAI-2K;
No new organ systems affected as defined by 1 or more BILAG-2004 A or 2 or
more BILAG-2004 B items compared to baseline using BILAG-2004;
No worsening from baseline in the subjects' lupus disease activity defined by an
increase ≥0.30 points on a 3-point PGA VAS

### 5.8.5 SLEDAI-2K (Systemic Lupus Erythematosus Disease Activity Index 2000)

The SLEDAI-2K disease activity index consists of a list of organ manifestations, each with a definition. A certified Investigator or designated physician will complete the SLEDAI-2K assessment and decide whether each manifestation is “present” or “absent” in the last 4 weeks. The assessment also includes the collection of blood and urine for assessment of the laboratory categories of the SLEDAI-2K.

The SLEDAI-2K assessment consists of 24 lupus-related items. It is a weighted instrument, in which descriptors are multiplied by a particular organ's “weight”. For example, renal descriptors are multiplied by 4 and central nervous descriptors by 8 and these weighted organ manifestations are totaled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window⁴⁵.

SLEDAI-2K-defined resolution of rash is defined as a score of 0 at Week 52 for those with a score ≥2 for rash at baseline.

### 5.9 Type I IFN Gene Signature (IFNGS)

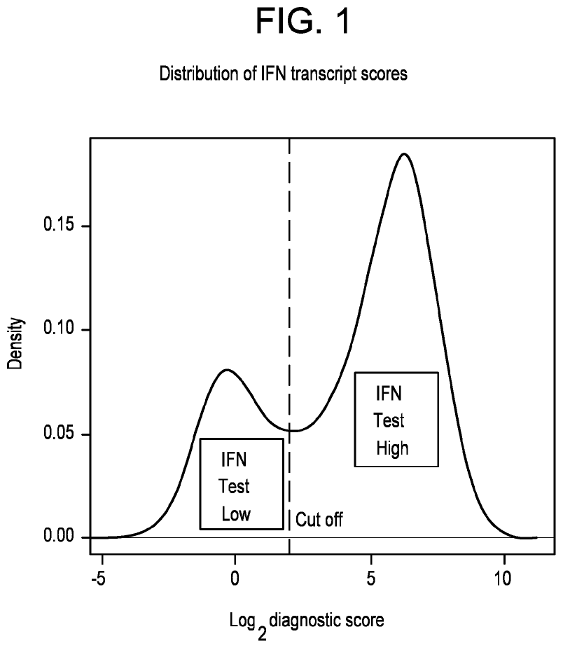

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1). The type I IFN test is described in WO2011028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IFI27, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression (FIG. 1).

The expression of the genes may be measured by RT-PCR. Suitable primers and probes for detection of the genes may be found in WO2011028933. A suitable kit for measuring gene expression for the IFNGS test is the QIAGEN Therascreen® IFIGx RGQ RT-PCR kit (IFIGx kit), as described in Brohawn et al.⁴⁶, which is incorporated herein by reference in its entirety.

### 5.10 Type I IFN Gene Signature (IFNGS)

The Interferon Gene Signature (IFNGS) is defined as a set of specific gene transcripts whose expression increases once the IFN receptor (IFNAR1) gets activated by binding of Type I IFN ligands (IFN-α, IFN-β and IFN-ω). Two Interferon Gene Signatures are used as part of the Saphnelo and sifalimumab trials to provide different readouts: The 4-genes Interferon Gene Signature is a peripheral blood signature that was derived from genome-wide gene expression studies and further validated by a quantitative PCT test (developed to specifically measure IFN gene expression based on 4 genes). It is further used at baseline to understand whether a disease or a particular patient's disease is type I IFN driven. The 21 Interferon Gene Signature is a peripheral blood signature that was derived from genome-wide gene expression studies. It is used to study the pharmacodynamic effect of Saphnelo by providing a measure for Type 1 interferon signaling inhibition after treatment.

The IFN 21-gene signature (IFNGS) is a validated pharmacodynamic marker of type I IFN signaling, that is elevated in patients with type I IFN-mediated disease, including SLE, lupus nephritis, myositis, Sjogren's and scleroderma.

A 4-gene IFNGS score is calculated by measurement of IFI27, IFI44, IFI44L, and RSAD2 expression. A 5-gene IFNGS score is calculated by measurement of IFI27, RSAD2, IFI44, IFI44L, IFI6 expression. A 21-gene IFNGS score is calculated by measurement of the genes shown in Table 5. Gene expression may be measured by detecting mRNA in the whole blood or tissue of the subject. A IFNGS (4-gene, 5-gene or 21-gene) score may be detected in a subject by measuring the IFNGS gene expression (e.g. mRNA) in the blood or tissue of the subject and comparing the gene expression levels to expression of house-keeping or control genes, e.g. ACTB, GAPDH, and 18S rRNA, in the blood or tissue.

TABLE 5
21-gene IFNGS

| | Gene | Gene |
|---|---|---|
| Gene title | symbol | Probe ID |
| Interferon, alpha-inducible protein 27 | IFI27 | 202411 |
| Interferon, alpha-inducible protein 6 | IFI6 | 204415 |
| Radical S-adenosyl methionine domain | RSAD2 | 213797 |
| containing 2 | | |
| Interferon-induced protein 44 | IF144 | 214059 |
| Interferon-induced protein 44-like | IFI44L | 204439 |
| Ubiquitin specific peptidase 18 | USP18 | 219211 |
| Lymphocyte antigen 6 complex, locus E | LY6E | 202145 |
| 2,5-oligoadenylate synthetase 1, 40/46 kDa | OAS1 | 202869 |
| Sialic acid binding Ig-like lectin 1, sialoadhesin | SIGLECI | 44673 |
| ISG15 ubiquitin-like modifier | ISG15 | 205483 |
| Interferon-induced protein with tetratricopeptide | IFIT1 | 203153 |
| repeats 1 | | |
| 2′-5′-oligoadenylate synthetase 3, 100 kDa | OAS3 | 218400 |
| Hect domain and RLD 5 | HERC5 | 219863 |
| Myxovirus (influenza virus) resistance 1 | MX1 | 202086 |
| Lysosomal-associated membrane protein 3 | LAMP3 | 205569 |
| Epithelial stromal interaction 1 (breast) | EPSTI1 | 227609 |
| Interferon-induced protein with tetratricopeptide | IFIT3 | 204747 |
| repeats 3 | | |
| 2′-5′-oligoadenylate synthetase 2, 69/71 kDa | OAS2 | 204972 |
| Receptor (chemosensory) transporter protein 4 | RTP4 | 219684 |
| Phospholipid scramblase 1 | PLSCR1 | 241916 |
| DNA polymerase-transactivated protein 6 | DNAPTP6 | 241812 |

### 6 Example 1: MUSE, ClinicalTrial.Gov Identifier: NCT01438489

MUSE was a Phase 2, multinational, multicentre, randomized, double-blind, placebo controlled, parallel-group study to evaluate the efficacy and safety of 2 intravenous (IV) treatment regimens in adult participants with chronic, moderately-to-severely active SLE with an inadequate response to standard of care (SOC) SLE. The investigational product (anifrolumab or placebo) was administered as a fixed dose every 4 weeks (28 days) for a total of 13 doses.

MUSE is described in further detail in Furie et al. 201733, which is incorporated herein by reference in its entirety.

### 7 Example 2: TULIP I and II, ClinicalTrial.Gov Identifiers: NCT02446912 and NCT02446899

TULIP I and TULIP II were Phase 3, multicentre, multinational, randomised, double-blind, placebo-controlled studies to evaluate the efficacy and safety of an intravenous (IV) treatment regimen of two doses of anifrolumab versus placebo in subjects with moderately to severely active, autoantibody-positive systemic lupus erythematosus (SLE) while receiving standard of care (SOC) treatment.

### 7.1.1 Restricted Medications

If a subject received 1 of the following, the subject was considered a non-responder. Sulfasalazine; Danazol; Dapsone; Azathioprine >200 mg/day or at a daily dose greater than that at Week 0 (Day 1); Mycophenolate mofetil >2.0 g/day or mycophenolic acid >1.44 g/day or at a daily; dose greater than that at Week 0 (Day 1); Oral, SC, or intramuscular methotrexate >25 mg/week or at a daily dose greater than that at Week 0 (Day 1); Mizoribine >150 mg/day or at a daily dose greater than that at Week 0 (Day 1); Any change in route of administration of oral, SC, or intramuscular methotrexate; Intravenous corticosteroids >40 mg/day but ≤1 gm/day methylprednisolone or equivalent; Intramuscular corticosteroids >80 mg/day methylprednisolone or equivalent; Subcutaneous or intramuscular corticosteroid precursors; Treatment with OCS >40 mg/day prednisone or equivalent; Treatment with OCS above Day 1 dose for a dosing period >14 days; Corticosteroids with a long biologic half-life (eg, dexamethasone, betamethasone); Other immunosuppressants including but not limited to calcineurin inhibitors (eg, cyclosporine, tacrolimus [including topical]) or leflunomide. Cyclosporine eye drops were acceptable for use in the study.

TULIP I is described in further detail in Furie et al. 201934, which is incorporated herein by reference in its entirety. The results of TULIP II are presented in Morand et al. 202032, herein incorporated by reference in its entirety.

### 8 Example 3: Steroid Tapering

### 8.1 Summary

### 8.1.1 Background and Objectives

Glucocorticoids are a mainstay of systemic lupus erythematosus (SLE) treatment despite their association with significant toxicity. Therefore, a priority SLE treatment goal is to reduce glucocorticoid use. Glucocorticoid sparing is a key priority for systemic lupus erythematosus (SLE) management. The inventors analysed pooled data from the TULIP-1 and TULIP-2 phase 3 trials in patients with moderate to severe SLE to assess anifrolumab's effect on glucocorticoid tapering.

### 8.1.2 Methods

TULIP-1 and TULIP-2 were randomized, placebo-controlled, 52-week trials of intravenous anifrolumab (300 mg every 4 weeks for 48 weeks). The inventors evaluated changes in glucocorticoid dosage, clinical and laboratory assessments, patient-reported outcomes (PROs), and safety in patients receiving ≥10 mg/day glucocorticoids at baseline by treatment group and by glucocorticoid taper response, regardless of treatment group. In a pooled cohort of patients receiving ≥10 mg/day glucocorticoids at baseline, the inventors evaluated changes in glucocorticoid dosage, patient-reported outcomes (PROs), and safety in patients who achieved a sustained glucocorticoid taper response, defined as achieving $7.5 mg/day by Week 40 and sustained to Week 52.

### 8.1.3 Results

A total of 50.5% (96/190) patients receiving ≥10 mg/day glucocorticoids at baseline and treated with anifrolumab achieved sustained glucocorticoid reduction (≤7.5 mg/day, Weeks 40-52; glucocorticoid responder) vs 31.8% (59/185) with placebo (nominal P<0.001). The mean cumulative glucocorticoid dose was reduced by 8% with anifrolumab vs placebo and by 44% for glucocorticoid responders vs nonresponders. Most patients classified as anifrolumab-treatment responders (by British Isles Lupus Assessment Group-based Composite Lupus Assessment) were also glucocorticoid responders (80% [72/89]). Safety was similar across groups. However, glucocorticoid nonresponders reported more serious adverse events.

### 8.1.4 Conclusions

Anifrolumab improved disease activity while reducing glucocorticoid dosage. Glucocorticoid tapering is also be associated with additional health benefits. In patients with moderate to severe SLE, sustained glucocorticoid tapering is associated with improvements in PROs, blood pressure, and fewer SAEs. Together with the higher rates of glucocorticoid tapering in patients treated with anifrolumab, these results illustrate the ability of anifrolumab to reduce glucocorticoid-associated adverse effects, a key goal of SLE management.

### 8.2 Introduction

Glucocorticoids are used in up to 80% of patients with SLE; the majority being treated long-term. Despite their short-term benefits, glucocorticoids are associated with a significant burden of toxicity. Compared with patients not taking glucocorticoids, SLE patients taking a mean prednisone dosage >7.5 mg/day over a period of 4 years had a nearly 10-fold increased risk of organ damage, including cataracts, osteoporotic fractures, diabetes mellitus, and cardiovascular disease. By contrast, daily doses $7.5 mg/day are associated with fewer adverse effects.

Compared with no glucocorticoid use in patients with SLE, mean prednisone dosages >7.5 mg/day over a period of 4 years were associated with a nearly 10-fold increased risk of organ damage, including cataracts, osteoporotic fractures, diabetes mellitus, and cardiovascular disease. By contrast, daily doses ≤7.5 mg are associated with fewer adverse effects, and this prednisone dose threshold is used in the definition of the lupus low disease activity state, which is associated with a lower risk of adverse outcomes, though patients with low lupus disease activity who are treated with prednisone, even at low doses, can still experience poor emotional health. Therefore, novel, effective, and long-term treatments for SLE are needed to both reduce overall disease activity and glucocorticoid use.

In this analysis of pooled data from the TULIP-1 and TULIP-2 trials, we further investigated the effect of anifrolumab treatment compared with placebo on glucocorticoid dose reduction. In addition, the inventors explored whether there were any changes associated with glucocorticoid reduction with regard to patient-reported outcomes (PROs), clinical and laboratory values, serious adverse events (SAEs), and cardiovascular adverse events (AEs). Analyses were conducted by both treatment group and by treatment agnostic grouping (patients who were able to taper glucocorticoids ≤7.5 mg/day; glucocorticoid responders) to better define the potential health benefits of glucocorticoid dose tapering.

### 8.3 Methods

### 8.3.1 Patients and Study Design

This was a post hoc analysis of pooled data from the 52-week TULIP-1 and TULIP-2 trials of anifrolumab in which patients with moderate to severe SLE, despite standard therapy with glucocorticoids, antimalarials, and/or immunosuppressants, were randomized to receive intravenous infusions of anifrolumab 300 mg or placebo every 4 weeks for 48 weeks. The study design and methods have been described in detail previously^{32,34}. In brief, eligible patients were 18 to 70 years of age and fulfilled the American College of Rheumatology 1997 classification criteria for SLE. For patients receiving oral glucocorticoid ≥10 mg/day (prednisone or equivalent) at baseline, a protocol-mandated attempt to taper to ≤7.5 mg/day was required between Weeks 8 and 40; tapering was also permitted for patients receiving oral glucocorticoid <10 mg/day at baseline. Stable oral glucocorticoid dose was required in all patients between Weeks 40 and 52.

### 8.3.2 Study Endpoints and Assessments

In this analysis, the inventors evaluated the prespecified secondary endpoint of sustained glucocorticoid dosage reduction at Week 52 in pooled data from TULIP-1 and TULIP-2 for patients receiving baseline glucocorticoid ≥10 mg/day. Analyses included only patients receiving baseline glucocorticoid ≥10 mg/day randomized to receive anifrolumab 300 mg or placebo; the anifrolumab 150 mg group in TULIP-1 was excluded from these analyses. Pooled patient data were evaluated by both treatment group and/or by glucocorticoid tapering response, regardless of treatment group assignment. Glucocorticoid responders were defined as achieving an oral glucocorticoid dosage ≤7.5 mg/day by Week 40, having stable glucocorticoid dosage from Week 40 through Week 52, and having no permanent premature discontinuation of investigational product or use of restricted medications beyond the protocol-allowed threshold. If any of the conditions could not be evaluated at Week 52 (eg, owing to missing values), the patient was defined as a nonresponder.

### 8.3.3 Assessment of Outcomes in Anifrolumab and Placebo Treatment Groups

Outcome measures were compared between patients randomized to receive anifrolumab 300 mg and placebo, including the percentage of patients achieving sustained oral glucocorticoid dose reduction, least squares (LS) mean changes to baseline glucocorticoid daily dose, and cumulative dosage of glucocorticoids measured by the mean area under the curve (AUC). Changes in PROs were assessed from baseline to Week 52, including responses in Functional Assessment of Chronic Illness Therapy-Fatigue [FACIT-F] (defined as a >3-point improvement), responses in Short Form 36 Health Survey version 2 [SF-36-v2] [acute] physical component summary [PCS] and mental component summary [MCS] (defined as an improvement of >3.4 in the PCS and >4.6 in the MCS). LS mean changes were assed from baseline to Week 24 and Week 52 in weight, body mass index (BMI), fasting glucose, cholesterol, hematologic values (hematocrit, erythrocytes, leukocytes, lymphocytes, neutrophils, and platelets), as well as cardiovascular measures (diastolic and systolic blood pressure and heart rate). Serious adverse events (SAEs) and cardiovascular adverse events (AEs) were also assessed.

### 8.3.4 Outcomes Assessed in Anifrolumab 300 mg and Placebo Treatment Groups

Outcome measures were also compared between patients receiving baseline glucocorticoids ≥10 mg/day randomised to receive anifrolumab 300 mg and placebo, including the percentage of patients achieving a sustained glucocorticoid taper response, LS mean changes from baseline glucocorticoid daily dose, cumulative dose of glucocorticoids, improvement responses in PROs, and safety. Additional analyses of the percentage of sustained glucocorticoid taper responders and British Isles Lupus Assessment Group (BILAG)-based Composite Lupus Assessment (BICLA) responders, as defined in the TULIP trials, were compared between treatment groups.

### 8.3.5 Assessment of Outcomes in Glucocorticoid Responders and Nonresponders

Cumulative dosage of glucocorticoids, PROs, clinical and laboratory values, and safety were also compared between glucocorticoid responders and nonresponders at Week 52, regardless of treatment group assignment. Additionally, percentage of glucocorticoid responders and British Isles Lupus Assessment Group (BILAG)-based Composite Lupus Assessment (BICLA) responders were compared between treatment groups. BICLA response was defined as reduction of all baseline BILAG-2004 A and B scores and no worsening in other organ systems, no worsening from baseline in SLEDAI-2K, and no increase ≥0.30 points on a 3-point Patient's Global Assessment visual analog scale from baseline.

### 8.3.6 Statistical Analysis

The similar designs of the TULIP-1 and TULIP-2 studies allowed for the results to be pooled. Sample sizes were selected for TULIP-1 and TULIP-2 based on powering of the primary and key secondary endpoints and to ensure an adequate safety database. In TULIP-1 and TULIP-2, 180 patients/arm yielded >99% and 88% power, respectively, to reject the hypothesis (no difference in the primary endpoint) using a 2-sided alpha of 0.05. Changes from baseline were analyzed using a mixed model with repeated measures (MMRM), responder vs nonresponder rates were calculated using a stratified Cochran-Mantel-Haenszel approach, and glucocorticoid AUC was analyzed with an analysis of covariance model. The models included fixed effects and stratification factors for baseline value, including oral glucocorticoid dosage (<10 mg/day or ≥10 mg/day), treatment group, visit (including study for the pooled analysis), treatment visit interaction and stratification factors (SLEDAI-2K score at screening [<10 points vs ≥10 points] and type 1 IFN gene signature test result at screening [high vs low]). All P-values, 95% Cls, and standard errors are based on these models. As these analyses were not part of the formal testing strategy, all P-values are nominal. Missing data were imputed using the last observation carried forward for the first visit with missing data; subsequent visits with missing data were not imputed.

### 8.4 Results

### 8.4.1 Patient Demographics and Clinical Characteristics

Across the 2 TULIP studies, 726 patients were randomized to receive anifrolumab 300 mg (n=360 [180 patients in each trial]) or placebo (n=366 [184 and 182 patients in TULIP-1 and TULIP-2, respectively]). Most patients 595/726 (82%) were receiving oral glucocorticoids (prednisone or equivalent) at baseline, of whom 375 were receiving ≥10 mg/day (n=190, anifrolumab; n=185 placebo), with a mean daily dose of 15.2 mg for both treatment groups. Patient demographics and baseline clinical characteristics were comparable between treatment groups of patients receiving baseline glucocorticoid ≥10 mg/day from the pooled TULIP trials (Table 8-1).

TABLE 8-1
Patient demographics and baseline clinical characteristics in patients receiving

| | Patients with a baseline glucocorticoid dosage >10 mg/day (n=375) | | | |
|---|---|---|---|---|
| | | | Glucocorticoid | Glucocorticoid |
| | Placebo | Anifrolumab | responders^{a} | nonresponders^{a} |
| Baseline characteristic | (n = 185) | (n = 190) | (n = 155) | (n = 220) |
| Age, mean (SD), years | 39.0 (11.15) | 39.7 (11.62) | 40.2 (11.54) | 38.7 (11.25) |
| Female, n (%) | 170 (91.9) | 172 (90.5) | 140 (90.3) | 202 (91.8) |
| Race, n (%) | | | | |
| White | 131 (70.8) | 116 (61.1) | 101 (65.2) | 146 (66.4) |
| Black or African American | 19 (10.3) | 27 (14.2) | 15 (9.7) | 31 (14.1) |
| Asian | 15 (8.1) | 23 (12.1) | 19 (12.3) | 19 (8.6) |
| Native Hawaiian or Other | 0 | 0 | 0 | 0 |
| Pacific | | | | |
| Islander | | | | |
| American Indian or Alaska | 0 | 0 | 0 | 0 |
| Native | | | | |
| Other | 17 (9.2) | 20 (10.5) | 17 (11.0) | 20 (9.1) |
| Ethnic group, n (%) | | | | |
| Hispanic or Latino | 45 (24.3) | 49 (25.8) | 40 (25.8) | 54 (24.5) |
| IFNGS high at screening, n | 160 (86.5) | 168 (88.4) | 134 (86.5) | 194 (88.2) |
| (%) | | | | |
| Time from SLE diagnosis | 83.0 (4-494) | 97.0 (6-493) | 84.0 (6-450) | 88.0 (4-494) |
| to randomization, median | | | | |
| (range), months | | | | |
| BILAG-2004 | | | | |
| ≥1 A item, n (%) | 87 (47.0) | 98 (51.6) | 80 (51.6) | 105 (47.7) |
| No A and ≥2 B items, n (%) | 82 (44.3) | 81 (42.6) | 68 (43.9) | 95 (43.2) |
| No A and <2 B items, n (%) | 16 (8.6) | 11 (5.8) | 7 (4.5) | 20 (9.1) |
| SLEDAI-2K score, mean | 11.9 (3.99) | 11.6 (3.74) | 11.2 (3.21) | 12.2 (4.21) |
| (SD) | | | | |
| <10, n (%) | 44 (23.8) | 53 (27.9) | 44 (28.4) | 53 (24.1) |
| ≥10, n (%) | 141 (76.2) | 137 (72.1) | 111 (71.6) | 167 (75.9) |
| PGA score, mean (SD) | 1.91 (0.36) | 1.84 (0.44) | 1.80 (0.43) | 1.93 (0.37) |
| CLASI activity score, | 8.0 (6.44) | 9.6 (8.54) | 9.5 (7.68) | 8.4 (7.55) |
| mean (SD) | | | | |
| <10, n (%) | 126 (68.1) | 121 (63.7) | 94 (60.6) | 153 (69.5) |
| ≥10, n (%) | 69 (36.3) | 59 (31.9) | 61 (39.4) | 67 (30.5) |
| 0, n (%) | 12 (6.5) | 7 (3.7) | 8 (5.2) | 11 (5.0) |
| >0, n (%) | 173 (93.5) | 183 (96.3) | 147 (94.8) | 209 (95.0) |
| SDI global score, mean | 0.5 (0.80) | 0.6 (1.00) | 0.5 (0.94) | 0.6 (0.89) |
| (SD) | | | | |
| Swollen joint count, | 7.3 (5.93) | 6.2 (5.33) | 6.1 (4.93) | 7.3 (6.07) |
| mean (SD) | | | | |
| Tender joint count, mean | 10.7 (7.65) | 10.0 (7.49) | 10.3 (7.29) | 10.4 (7.78) |
| (SD) | | | | |
| Oral glucocorticoid use^{b} | | | | |
| Mean (SD) | 15.21 (7.52) | 15.21 (10.44) | 13.49 (5.87) | 16.42 (10.67) |
| Glucocorticoid ≥10 mg/day, | 185 (100) | 190 (100) | 155 (100) | 220 (100) |
| n (%) | | | | |
| Oral glucocorticoid only, n | 28 (15.1) | 42 (22.1) | 29 (18.7) | 41 (18.6) |
| (%) | | | | |
| Oral glucocorticoid only, | 15.71 (7.29) | 13.69 (5.16) | 12.50 (4.53) | 15.91 (6.75) |
| mean (SD) | | | | |
| Oral glucocorticoid with | 157 (84.9) | 148 (77.9) | 126 (81.3) | 179 (81.4) |
| antimalarials and/or | | | | |
| immunosuppressants, n | | | | |
| (%) | | | | |
| Mean (SD) | 15.12 (7.58) | 15.64 (11.49) | 13.72 (6.12) | 16.54 (11.40) |
| Time on glucocorticoid up | 5.16 (0-398) | 4.83 (0-310) | 4.90 (0-198) | 5.14 (0-398) |
| to randomization, median | | | | |
| (range), months | | | | |
| Vital signs, mean (SD) | | | | |
| Diastolic, sitting blood | 74.68 (9.64) | 74.56 (8.945) | 75.20 (9.17) | 74.21 (9.37) |
| pressuremm Hg | | | | |
| Systolic sitting blood | 119.27 (13.84) | 118.72 (13.13) | 119.33 (12.84) | 118.75 (13.92) |
| pressure, mm Hg | | | | |
| Heart rate, beats/min | 75.50 (10.94) | 75.71 (11.68) | 74.17 (10.99) | 76.62 (11.44) |
| Laboratory parameters, | | | | |
| mean (SD) | | | | |
| Weight, kg | 70.46 (16.62) | 71.79 (18.93) | 69.69 (16.78) | 72.15 (18.48) |
| BMI, kg/m² | 26.31 (5.82) | 27.25 (6.63) | 26.61 (5.93) | 26.91 (6.49) |
| Fasting glucose, mmol/L | 4.77 (0.76) | 4.85 (1.05) | 4.87 (1.04) | 4.78 (0.81) |
| Total cholesterol, mmol/L | 5.01 (1.12) | 4.98 (1.12) | 4.95 (1.11) | 5.03 (1.12) |
| HDL, mmol/L | 1.48 (0.42) | 1.52 (0.49) | 1.52 (0.45) | 1.48 (0.46) |
| LDL, mmol/L | 2.83 (0.92) | 2.73 (0.88) | 2.71 (0.87) | 2.83 (0.92) |
| Triglycerides, mmol/L | 1.51 (0.72) | 1.57 (0.85) | 1.54 (0.84) | 1.55 (0.75) |
| Hematocrit | 0.38 (0.05) | 0.38 (0.05) | 0.39 (0.04) | 0.38 (0.05) |
| Erythrocytes, 10¹²/L | 4.27 (0.50) | 4.23 (0.51) | 4.31 (0.49) | 4.21 (0.51) |
| Leukocytes, 10⁹/L | 6.17 (2.58) | 5.81 (2.48) | 5.73 (2.26) | 6.16 (2.70) |
| Lymphocytes, 10⁹/L | 1.28 (0.68) | 1.26 (0.73) | 1.29 (0.68) | 1.26 (0.72) |
| Neutrophils, 10⁹/L | 4.45 (2.28) | 4.10 (2.04) | 3.98 (1.85) | 4.47 (2.34) |
| Platelets, 10⁹/L | 258.67 (86.14) | 240.59 (80.12) | 242.17 (79.41) | 254.68 (86.11) |

BILAG-2004, British Isles Lupus Assessment Group-2004;
BMI, body mass index;
CLASI, Cutaneous Lupus Erythematosus Disease Area and Severity Index;
HDL, high-density lipoproteins;
IFNGS, interferon gene signature;
LDL, low-density lipoproteins;
PGA, Physician's Global Assessment;
SD, standard deviation;
SDI, Systemic Lupus International Collaborating Clinics/American College of Rheumatology Damage Index;
SLE, systemic lupus erythematosus;
SLEDAI-2K, SLE Disease Activity Index 2000.
^{a}Sustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day.
^{b}Oral glucocorticoid includes prednisone or equivalent.

Regardless of treatment group assignment, there were 155 patients classified as glucocorticoid responders and 220 as glucocorticoid nonresponders at Week 52 among patients receiving baseline glucocorticoid ≥10 mg/day. Patient demographics and clinical characteristics were also similar between the glucocorticoid responder and nonresponder groups, though a greater proportion of Black/African American patients were glucocorticoid nonresponders and glucocorticoid nonresponders had a higher baseline mean daily glucocorticoid dose compared with responders (Table 8-1).

### 8.4.2 Outcomes for Sustained Glucocorticoid Taper Responders Vs Nonresponders

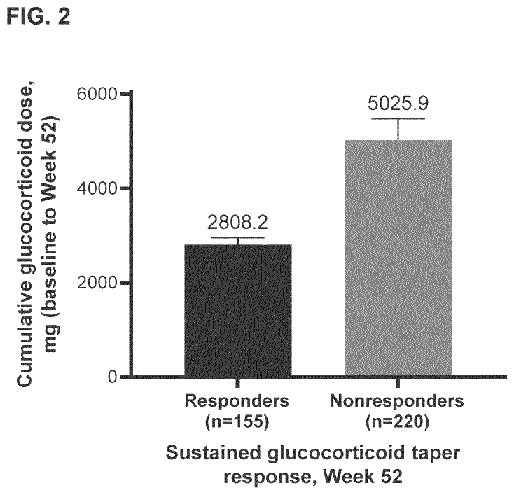

The mean cumulative dose of glucocorticoids during the 52 weeks of treatment was 44% lower among patients who were glucocorticoid taper responders vs nonresponders (mean [SE] AUC at Week 52:2808.2 [76.0] mg vs 5025.9 [231.7] mg) (FIG. 2). The sustained glucocorticoid taper responder group had more patients with clinically meaningful improvements in FACIT-F, SF-36 PCS, and SF-36 MCS scores (all P<0.001) compared with nonresponders (FIG. 3A-C).

Over the 52-week trials, the percentage of patients with ≥1 AE was 90.3% (140/155) of sustained glucocorticoid taper responders and 83.2% (183/220) of nonresponders. The incidence of serious AEs was 16.8% (26/155) in sustained glucocorticoid taper responders and 28.2% (62/220) in nonresponders (Table 8-2). Of serious AEs, serious infections, including pneumonia, were most commonly reported, occurring in 5.8% (9/155) of glucocorticoid responders and 13.2% (29/220) of nonresponders, and worsening of SLE was reported in 2.6% (4/155) of glucocorticoid taper responders and 5% (11/220) of nonresponders (Table 8-2). Cardiovascular AEs were reported in 12.3% (19/155) and 11.4% (25/220) of glucocorticoid taper responders and nonresponders, respectively (Table 8-3). Hypertension was the most common cardiovascular AE reported in both responders and nonresponders.

TABLE 8-2
SAEs during treatment by sustained glucocorticoid
taper response in TULIP-1 and TULIP-2 (pooled data)

| | Patients receiving glucocorticoid ≥10 mg/day at baseline | |
|---|---|---|
| | (n = 375) | |
| | Sustained glucocorticoid | Sustained glucocorticoid taper |
| | taper responders^{a} | nonresponders^{a} |
| SAEs, n (%) | (n = 155) | (n = 220) |
| Patients with any SAE | 26 (16.8) | 62 (28.2) |
| Systemic lupus erythematosus | 4 (2.6) | 11 (5.0) |
| Pneumonia | 2 (1.3) | 12 (5.5) |
| Influenza | 2 (1.3) | 0 |
| Herpes zoster | 1 (0.6) | 2 (0.9) |
| Coronary artery disease | 1 (0.6) | 1 (0.5) |
| Lupus nephritis | 1 (0.6) | 1 (0.5) |
| Osteonecrosis | 1 (0.6) | 1 (0.5) |
| Pulmonary embolism | 1 (0.6) | 1 (0.5) |
| Abortion spontaneous | 1 (0.6) | 0 |
| Acute coronary syndrome | 1 (0.6) | 0 |
| Arthritis | 1 (0.6) | 0 |
| Cervical dysplasia | 1 (0.6) | 0 |
| Chest pain | 1 (0.6) | 0 |
| Cholelithiasis | 1 (0.6) | 0 |
| Dyspnoea | 1 (0.6) | 0 |
| Erysipelas | 1 (0.6) | 0 |
| Facial bones fracture | 1 (0.6) | 0 |
| Herpes zoster disseminated | 1 (0.6) | 0 |
| Herpes zoster meningitis | 1 (0.6) | 0 |
| Humerus fracture | 1 (0.6) | 0 |
| Hypersensitivity | 1 (0.6) | 0 |
| Incarcerated hernia | 1 (0.6) | 0 |
| Esophageal stenosis | 1 (0.6) | 0 |
| Pleural effusion | 1 (0.6) | 0 |
| Pneumonia staphylococcal | 1 (0.6) | 0 |
| Post herpetic neuralgia | 1 (0.6) | 0 |
| Renal impairment | 1 (0.6) | 0 |
| Tendon rupture | 1 (0.6) | 0 |
| Tenosynovitis | 1 (0.6) | 0 |
| Upper limb fracture | 1 (0.6) | 0 |
| Urosepsis | 1 (0.6) | 0 |
| Urticaria | 1 (0.6) | 0 |
| Uterine prolapse | 1 (0.6) | 0 |
| Ventricular arrhythmia | 1 (0.6) | 0 |
| Acute kidney injury | 0 | 3 (1.4) |
| Pyelonephritis | 0 | 3 (1.4) |
| UNCODED | 0 | 2 (0.9) |
| Acute respiratory failure | 0 | 2 (0.9) |
| Asthma | 0 | 2 (0.9) |
| Bronchitis | 0 | 2 (0.9) |
| Syncope | 0 | 2 (0.9) |
| Urinary tract infection | 0 | 2 (0.9) |
| Abscess | 0 | 1 (0.5) |
| Abscess limb | 0 | 1 (0.5) |
| Anemia | 0 | 1 (0.5) |
| Atrial fibrillation | 0 | 1 (0.5) |
| B-cell lymphoma | 0 | 1 (0.5) |
| Cardiac failure | 0 | 1 (0.5) |
| Cellulitis | 0 | 1 (0.5) |
| Chronic kidney disease | 0 | 1 (0.5) |
| Colitis | 0 | 1 (0.5) |
| Conversion disorder | 0 | 1 (0.5) |
| Dengue fever | 0 | 1 (0.5) |
| Endometrial hypertrophy | 0 | 1 (0.5) |
| Fall | 0 | 1 (0.5) |
| Gastroenteritis | 0 | 1 (0.5) |
| Gastroesophageal reflux disease | 0 | 1 (0.5) |
| Genital herpes | 0 | 1 (0.5) |
| Haemangioma of liver | 0 | 1 (0.5) |
| Haemorrhoidal haemorrhage | 0 | 1 (0.5) |
| Hydronephrosis | 0 | 1 (0.5) |
| Hypercalcemia | 0 | 1 (0.5) |
| Hypoesthesia | 0 | 1 (0.5) |
| Hypotension | 0 | 1 (0.5) |
| Iron deficiency anaemia | 0 | 1 (0.5) |
| Large intestine infection | 0 | 1 (0.5) |
| Malignant hypertension | 0 | 1 (0.5) |
| Meningitis viral | 0 | 1 (0.5) |
| Musculoskeletal chest pain | 0 | 1 (0.5) |
| Myasthenia gravis | 0 | 1 (0.5) |
| Myocardial infarction | 0 | 1 (0.5) |
| Nephrolithiasis | 0 | 1 (0.5) |
| Neutropenia | 0 | 1 (0.5) |
| Noncardiac chest pain | 0 | 1 (0.5) |
| Pain | 0 | 1 (0.5) |
| Pelvic inflammatory disease | 0 | 1 (0.5) |
| Peritonsillar abscess | 0 | 1 (0.5) |
| Pneumonia bacterial | 0 | 1 (0.5) |
| Postprocedural complication | 0 | 1 (0.5) |
| Postoperative wound infection | 0 | 1 (0.5) |
| Pulmonary alveolar | 0 | 1 (0.5) |
| haemorrhage | | |
| Pyelonephritis acute | 0 | 1 (0.5) |
| Renal failure | 0 | 1 (0.5) |
| Respiratory failure | 0 | 1 (0.5) |
| Sepsis | 0 | 1 (0.5) |
| Septic shock | 0 | 1 (0.5) |
| Spinal compression fracture | 0 | 1 (0.5) |
| Spinal stenosis | 0 | 1 (0.5) |
| Streptococcal urinary tract | 0 | 1 (0.5) |
| infection | | |
| Supraventricular tachycardia | 0 | 1 (0.5) |
| Swelling face | 0 | 1 (0.5) |
| Synovial cyst | 0 | 1 (0.5) |
| Traumatic fracture | 0 | 1 (0.5) |
| Ulcerative keratitis | 0 | 1 (0.5) |
| Uterine cancer | 0 | 1 (0.5) |
| Uterovaginal prolapse | 0 | 1 (0.5) |
| Venous thrombosis limb | 0 | 1 (0.5) |
| Wound infection staphylococcal | 0 | 1 (0.5) |

SAE, serious adverse event.
^{a}Sustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day.

At Week 40, from when glucocorticoid dosage was required to be stable, sustained glucocorticoid taper responders had lower systolic and diastolic sitting blood pressure compared with nonresponders (P=0.023 and P<0.001, respectively); differences in diastolic (P=0.010) but not systolic (P=0.381) sitting blood pressure were maintained at Week 52 (Table 8-4). During the 52-week trials, fewer sustained glucocorticoid taper responders compared with nonresponders started new supplementary blood pressure medications (7.5% [11/155] vs 15.9% [35/220]) (P=0.029).

TABLE 8-3
Cardiovascular AEs during treatment by sustained glucocorticoid taper
response in TULIP-1 and TULIP-2 (pooled data)

| | Patients receiving glucocorticoid ≥10 mg/ | |
|---|---|---|
| | day at baseline (n = 375) | |
| | Sustained glucocorticoid taper | Sustained glucocorticoid taper |
| | responders^{a} | nonresponders^{a} |
| AEs, n (%) | (n = 155) | (n = 220) |
| Patients with any AE | 19 (12.3) | 25 (11.4) |
| Hypertension | 5 (3.2) | 10 (4.5) |
| Essential hypertension | 1 (0.6) | 0 |
| Hypotension | 0 | 1 (0.5) |
| Thrombosis | 0 | 1 (0.5) |
| Vasodilation | 0 | 1 (0.5) |
| Venous thrombosis limb | 0 | 1 (0.5) |
| Palpitations | 1 (0.6) | 2 (0.9) |
| Coronary artery disease | 1 (0.6) | 1 (0.5) |
| Sinus bradycardia | 1 (0.6) | 1 (0.5) |
| Acute coronary syndrome | 1 (0.6) | 0 |
| Bradycardia | 1 (0.6) | 0 |
| Bundle branch block right | 1 (0.6) | 0 |
| Left ventricular dilation | 1 (0.6) | 0 |
| Ventricular arrhythmia | 1 (0.6) | 0 |
| Supraventricular tachycardia | 0 | 2 (0.9) |
| Tachycardia | 0 | 2 (0.9) |
| Atrial fibrillation | 0 | 1 (0.5) |
| Cardiac failure | 0 | 1 (0.5) |
| Cardiomyopathy | 0 | 1 (0.5) |
| Myocardial infarction | 0 | 1 (0.5) |
| Syncope | 0 | 4 (1.8) |

AE, adverse event.
^{a}Sustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day.

Mean changes in weight, BMI, fasting glucose, cholesterol, and laboratory haematological values are shown in Table 8-5. At Week 52, mean changes were generally similar between glucocorticoid responders and nonresponders. Both groups had moderate increases in weight and BMI from baseline to Week 52. Glucocorticoid responders also had moderate decreases in triglycerides at Week 52 compared with nonresponders who had no change (Table 8-5).

TABLE 8-4
Change in blood pressure and pulse rate at Week 40 and Week 52 by sustained
glucocorticoid taper response in TULIP-1 and TULIP-2 (pooled data)

| Patients receiving glucocorticoid ≥10 mg/ | |
|---|---|
| day at baseline (n = 375) | |
| Sustained glucocorticoid | Sustained glucocorticoid |
| taper responders^{a} | taper nonresponders^{a} |
| (n = 155) | (n = 220) |

Systolic, sitting blood pressure, mm Hg

| Baseline, mean (SD) | 119.3 | (12.8) | 118.8 | (13.9) |
|---|---|---|---|---|
| Week 40, change from baseline, | −2.3 | (1.2) | 0.9 | (1.1) |
| LS mean (SE) | | | | |
| Difference, LS mean (95% CI) | −3.2 (−5.9, −0.4) | | | |
| Nominal P-value | 0.023 | | | |
| Week 52, change from baseline, | −0.1 | (1.2) | 1.2 | (1.2) |
| LS mean (SE) | | | | |

| | |
|---|---|
| Difference, LS mean (95% CI) | −1.3 (−4.1, 1.6) |
| Nominal P-value | 0.381 |

Diastolic, sitting blood pressure, mm Hg

| Baseline, mean (SD) | 75.2 | (9.2) | 74.2 | (9.4) |
|---|---|---|---|---|
| Week 40, change from baseline, | −2.4 | (0.8) | 1.0 | (0.8) |
| LS mean (SE) | | | | |
| Difference, LS mean (95% CI) | −3.4 (−5.3, −1.5) | | | |
| Nominal P-value | <0.001 | | | |
| Week 52, change from baseline, | −1.2 | (0.8) | 1.6 | (0.8) |
| LS mean (SE) | | | | |

| | |
|---|---|
| Difference, LS mean (95% CI) | −2.7 (−4.8, −0.7) |
| Nominal P-value | 0.010 |

Pulse rate, beats/min

| Baseline, mean (SD) | 74.2 | (11.0) | 76.6 | (11.4) |
|---|---|---|---|---|
| Week 40, change from baseline, | −1.1 | (0.8) | 0.7 | (0.8) |
| LS mean (SE) | | | | |
| Difference, LS mean (95% CI) | −1.8 (−3.8, 0.2) | | | |
| Nominal P-value | 0.080 | | | |
| Week 52, change from baseline, | −1.6 | (0.8) | −1.1 | (0.8) |
| LS mean (SE) | | | | |

| | |
|---|---|
| Difference, LS mean (95% CI) | −0.5 (−2.48, 1.47) |
| Nominal P-value | 0.615 |

CI, confidence interval;
LS, least squares;
SD, standard deviation;
SE, standard error.
^{a}Sustained glucocorticoid taper responder defined as a glucocorticoid dosage reduction to ≤7.5 mg/day by Week 40 without a dosage increase between Week 40 and Week 52 in patients with a baseline glucocorticoid dosage ≥10 mg/day.

TABLE 8-5
Changes from baseline in laboratory values at Week 52 by sustained
glucocorticoid taper response in TULIP-1 and TULIP-2 (pooled data)

| | Patients receiving glucocorticoid ≥10 mg/ | | | |
|---|---|---|---|---|
| | day at baseline (n = 375) | | | |
| | Sustained glucocorticoid | | Sustained glucocorticoid taper | |
| | taper responders^{a} | | nonresponders^{a} | |
| Mean (SD) | (n = 155) | | (n = 220) | |
| Weight, kg, mean (SD) | | | | |
| Baseline | 69.7 | (16.8) | 72.2 | (18.5) |
| Week 52, change from baseline | 1.7 | (6.6) | 1.4 | (5.3) |
| BMI, kg/m², mean (SD) | | | | |
| Baseline | 26.6 | (5.9) | 26.9 | (6.5) |
| Week 52, change from baseline | 0.6 | (2.5) | 0.5 | (2.0) |
| Fasting glucose, | | | | |
| mmol/L, mean (SD) | | | | |
| Baseline | 4.9 | (1.0) | 4.8 | (0.8) |
| Week 52, change from baseline | 0.1 | (1.1) | 0.0 | (0.9) |
| Total cholesterol, | | | | |
| mmol/L, mean (SD) | | | | |
| Baseline | 5.0 | (1.1) | 5.0 | (1.1) |
| Week 52, change from baseline | −0.1 | (0.8) | −0.0 | (0.9) |
| HDL, mmol/L, mean (SD) | | | | |
| Baseline | 1.5 | (0.5) | 1.5 | (0.5) |
| Week 52, change from baseline | −0.0 | (0.3) | −0.0 | (0.3) |
| LDL, mmol/L | | | | |
| Baseline | 2.7 | (0.9) | 2.8 | (0.9) |
| Week 52, change from baseline | −0.0 | (0.7) | −0.0 | (0.7) |
| Triglycerides, mmol/L | | | | |
| Baseline | 1.5 | (0.8) | 1.6 | (0.7) |
| Week 52, change from baseline | −0.1 | (0.8) | 0.0 | (0.6) |
| Haematocrit | | | | |
| Baseline | 0.4 | (0.0) | 0.4 | (0.0) |
| Week 52, change from baseline | −0.0 | (0.0) | 0.0 | (0.0) |
| Erythrocytes, 10¹²/L | | | | |
| Baseline | 4.3 | (0.5) | 4.2 | (0.5) |
| Week 52, change from baseline | 0.1 | (0.3) | 0.1 | (0.4) |
| Leukocytes, 10⁹/L | | | | |
| Baseline | 5.7 | (2.3) | 6.2 | (2.7) |
| Week 52, change from baseline | 0.6 | (2.3) | 0.4 | (2.7) |
| Lymphocytes, 10⁹/L | | | | |
| Baseline | 1.3 | (0.7) | 1.3 | (0.7) |
| Week 52, change from baseline | 0.2 | (0.7) | 0.1 | (0.7) |
| Neutrophils, 10⁹/L | | | | |
| Baseline | 4.0 | (1.9) | 4.5 | (2.3) |
| Week 52, change from baseline | 0.3 | (2.1) | 0.2 | (2.5) |
| Platelets, 10⁹/L | | | | |
| Baseline | 242.2 | (79.4) | 254.7 | (86.1) |
| Week 52, change from baseline | 19.2 | (57.0) | 9.5 | (60.1) |

BMI, body mass index;
HDL, high-density lipoprotein;
LDL, low-density lipoprotein;
SD, standard deviation.
^{a}Sustained glucocorticoid taper responder defined as a glucocorticoid dosage reduction to ≤7.5 mg/day by Week 40 without a dosage increase between Week 40 and Week 52 for patients receiving ≥10 mg/day at baseline.

### 8.4.3 Glucocorticoid Tapering

Patient demographics and baseline clinical characteristics were comparable between treatment groups in patients receiving baseline glucocorticoids ≥10 mg/day from the pooled cohort (Table 8-6).

TABLE 8-6
Patient demographics and baseline clinical characteristics by treatment group in patients
receiving glucocorticoid ≥10 mg/day at baseline in TULIP-1 and TULIP-2 (pooled data)

| | Patients receiving glucocorticoid ≥10 mg/ | | | |
|---|---|---|---|---|
| | day at baseline (n = 375) | | | |
| | Placebo | | Anifrolumab | |
| Baseline characteristic | (n = 185) | | (n = 190) | |
| Age, mean (SD), years | 39.0 | (11.2) | 39.7 | (11.6) |
| Female, n (%) | 170 | (91.9) | 172 | (90.5) |
| Race, n (%) | | | | |
| White | 131 | (70.8) | 116 | (61.1) |
| Black or African American | 19 | (10.3) | 27 | (14.2) |
| Asian | 15 | (8.1) | 23 | (12.1) |
| Other | 17 | (9.2) | 20 | (10.5) |
| Ethnic group, n (%) | | | | |
| Hispanic or Latino | 45 | (24.3) | 49 | (25.8) |
| IFNGS high at screening, n (%) | 160 | (86.5) | 168 | (88.4) |
| Time from SLE diagnosis to randomisation, | 83.0 | (4-494) | 97.0 | (6-493) |
| median (range), months | | | | |
| BILAG-2004 | | | | |
| ≥1 A item, n (%) | 87 | (47.0) | 98 | (51.6) |
| No A and ≥2 B items, n (%) | 82 | (44.3) | 81 | (42.6) |
| No A and <2 B items, n (%) | 16 | (8.6) | 11 | (5.8) |
| SLEDAI-2K score, mean (SD) | 11.9 | (4.0) | 11.6 | (3.7) |
| <10, n (%) | 44 | (23.8) | 53 | (27.9) |
| ≥10, n (%) | 141 | (76.2) | 137 | (72.1) |
| PGA score, mean (SD) | 1.9 | (0.4) | 1.8 | (0.4) |
| CLASI activity score, mean (SD) | 8.0 | (6.4) | 9.6 | (8.5) |
| <10, n (%) | 126 | (68.1) | 121 | (63.7) |
| ≥10, n (%) | 59 | (31.9) | 69 | (36.3) |
| 0, n (%) | 12 | (6.5) | 7 | (3.7) |
| >0, n (%) | 173 | (93.5) | 183 | (96.3) |
| SDI global score, mean (SD) | 0.5 | (0.8) | 0.6 | (1.0) |
| Swollen joint count, mean (SD) | 7.3 | (5.9) | 6.2 | (5.3) |
| Tender joint count, mean (SD) | 10.7 | (7.7) | 10.0 | (7.5) |
| Oral glucocorticoid use^{a} | | | | |
| Mean (SD) | 15.2 | (7.5) | 15.2 | (10.4) |
| Oral glucocorticoid ≥10 mg/day, n (%) | 185 | (100) | 190 | (100) |
| Oral glucocorticoid only, n (%) | 28 | (15.1) | 42 | (22.1) |
| Oral glucocorticoid only, mean (SD) | 15.7 | (7.3) | 13.7 | (5.2) |
| Oral glucocorticoid with antimalarials and/or | 157 | (84.9) | 148 | (77.9) |
| immunosuppressants, n (%) | | | | |
| Mean (SD) | 15.1 | (7.6) | 15.6 | (11.5) |
| Time on glucocorticoid up to randomisation, | 5.2 | (0-398) | 4.8 | (0-310) |
| median (range), months | | | | |
| Vital signs, mean (SD) | | | | |
| Systolic sitting blood pressure, mm Hg | 119.3 | (13.8) | 118.7 | (13.1) |
| Diastolic, sitting blood pressure, mm Hg | 74.7 | (9.6) | 74.6 | (8.95) |
| Pulse rate, beats/min | 75.5 | (10.9) | 75.7 | (11.7) |
| Laboratory parameters, mean (SD) | | | | |
| Weight, kg | 70.5 | (16.6) | 71.8 | (18.9) |
| BMI, kg/m² | 26.3 | (5.8) | 27.3 | (6.6) |
| Fasting glucose, mmol/L | 4.8 | (0.8) | 4.9 | (1.1) |
| Total cholesterol, mmol/L | 5.0 | (1.1) | 5.0 | (1.1) |
| HDL, mmol/L | 1.5 | (0.4) | 1.5 | (0.5) |
| LDL, mmol/L | 2.8 | (0.9) | 2.7 | (0.9) |
| Triglycerides, mmol/L | 1.5 | (0.7) | 1.57 | (0.9) |
| Haematocrit | 0.4 | (0.1) | 0.4 | (0.1) |
| Erythrocytes, 10¹²/L | 4.3 | (0.5) | 4.2 | (0.5) |
| Leukocytes, 10⁹/L | 6.2 | (2.6) | 5.8 | (2.5) |
| Lymphocytes, 10⁹/L | 1.3 | (0.7) | 1.3 | (0.7) |
| Neutrophils, 10⁹/L | 4.5 | (2.3) | 4.1 | (2.0) |
| Platelets, 10⁹/L | 258.7 | (86.1) | 240.6 | (80.1) |

BILAG-2004, British Isles Lupus Assessment Group-2004;
BMI, body mass index;
CLASI, Cutaneous Lupus Erythematosus Disease Area and Severity Index;
HDL, high-density lipoprotein;
IFNGS, interferon gene signature;
LDL, low-density lipoprotein;
PGA, Physician's Global Assessment;
SD, standard deviation;
SDI, Systemic Lupus International Collaborating Clinics/American College of Rheumatology Damage Index;
SLE, systemic lupus erythematosus;
SLEDAI-2K, SLE Disease Activity Index 2000.
^{a}Oral glucocorticoid includes prednisone or equivalent.

In the prespecified TULIP-1 and TULIP-2 trials secondary endpoint of glucocorticoid sustained reduction to $7.5 mg/day in patients receiving ≥10 mg/day at baseline, more patients in the pooled dataset receiving anifrolumab compared with placebo achieved a glucocorticoid response (50.5% [96/185] vs 31.8% [59/185]) (P<0.001). Using a more stringent threshold of glucocorticoid reduction to $5 mg/day, more patients also achieved sustained glucocorticoid reductions to $5 mg/day from Weeks 40 to 52 with anifrolumab compared with placebo (P=0.003) (FIG. 4A; FIG. 4B)

### 8.4.4 Glucocorticoid Dosage Changes During Study

The LS mean (SD) percentage reduction from baseline in the daily glucocorticoid dose was-42.5% (4.5) among patients in the anifrolumab group, compared with −27.7% (4.6) among those in the placebo group (LS mean difference −14.8%, 95% CI −27.17% to −2.42%, nominal P<0.019). More patients in the anifrolumab group than in the placebo group also had more stringent sustained glucocorticoid reduction from baseline between Week 40 and Week 52, including sustained glucocorticoid reductions of ≥25% (P<0.001), ≥50% (P=0.001), ≥75% (P=0.06), and ≥90% (P=0.09) (Table 8-7). Six patients in the anifrolumab group and 5 patients in the placebo group who reached an oral glucocorticoid dosage ≤7.5 mg/day at Week 40 increased their dosage to >7.5 mg/day after Week 40.

TABLE 8-7
Glucocorticoid dosage change from baseline in patients
receiving glucocorticoid ≥10 mg/day at
baseline in TULIP-1 and TULIP-2 (pooled data)

| | Placebo | | Anifrolumab |
|---|---|---|---|
| Dosage change by percent reduction | (n = 185) | | (n = 190) |
| Glucocorticoid reduction ≥90% | 7 | (3.6) | 17 (9.0) |
| at Week 52, n (%) | | | |
| Difference in response rate (95% CI) | 5.4 (−0.8, 11.6) | | |
| Nominal P-value | 0.09 | | |
| Glucocorticoid reduction ≥75% | 23 | (12.3) | 38 (20.0) |
| at Week 52, n (%) | | | |
| Difference in response rate (95% CI) | 7.7 (−0.2, 15.6) | | |
| Nominal P-value | 0.06 | | |
| Glucocorticoid reduction ≥50% | 43 | (23.2) | 73 (38.4) |
| at Week 52, n (%) | | | |
| Difference in response rate (95% CI) | 15.2 (5.9, 24.5) | | |
| Nominal P-value | 0.001 | | |
| Glucocorticoid reduction ≥25% | 59 | (31.8) | 96 (50.5) |
| at Week 52, n (%) | | | |

| | |
|---|---|
| Difference in response rate (95% CI) | 18.7 (8.9, 28.4) |
| Nominal P-value | <0.001 |

CI, confidence interval;
IFNGS, interferon gene signature;
SLEDAI-2K, Systemic Lupus Erythematosus Disease Activity Index 2000.
Difference in response rate was calculated using a stratified Cochran-Mantel-Haenszel approach, with stratification factors SLEDAI-2K score at screening (<10 points vs ≥10 points) and type I IFNGS test result at screening (high vs low). In the pooled analysis, an additional stratification factor is added for study (TULIP- 1 vs TULIP-2).

The mean cumulative dose of glucocorticoids during the 52 weeks of treatment was 8% lower in the anifrolumab group vs the placebo group (mean [SD] AUC at Week 52:3947.1 [3655.5] mg vs 4275.8 [1859.0] mg) and 44% lower among patients who were glucocorticoid responders vs nonresponders (mean [SD] AUC at Week 52:2808.2 [945.9] mg vs 5025.9 [3436.6] mg) (FIG. 4C, FIG. 4D).

The LS mean (SE) percentage reduction from baseline at Week 52 in the daily glucocorticoid dosage was-42.5% (4.5) among patients in the anifrolumab group, compared with −27.7% (4.7) among those in the placebo group (LS mean difference −14.8%, 95% CI −27.17%, −2.42%, P=0.021). More patients in the anifrolumab group than in the placebo group also had more stringent sustained glucocorticoid dosage reduction from baseline between Week 40 and Week 52, including sustained glucocorticoid reductions of ≥25% (P<0.001), ≥50% (P=0.001), ≥75% (P=0.057), and ≥90% (P=0.086) (Table 8-8).

TABLE 8-8
Sustained glucocorticoid dosage reduction from baseline between
Week 40 and Week 52 by treatment group in patients receiving
glucocorticoid ≥10 mg/day at baseline in TULIP-1 and TULIP-2 (pooled data)

| | Patients receiving glucocorticoid ≥10 mg/ | |
|---|---|---|
| | day at baseline (n = 375) | |
| | Placebo | Anifrolumab |
| Dosage change by percent reduction | (n = 185) | (n = 190) |
| Glucocorticoid reduction ≥90% | 7 (3.6) | 17 (9.0) |
| at Week 52, n (%) | | |
| Difference in response rate (95% CI) | 5.4 (−0.8, 11.6) | |
| Nominal P-value | 0.086 | |
| Glucocorticoid reduction ≥75% | 23 (12.3) | 38 (20.0) |
| at Week 52, n (%) | | |
| Difference in response rate (95% CI) | 7.7 (−0.2, 15.6) | |
| Nominal P-value | 0.057 | |
| Glucocorticoid reduction ≥50% at | 43 (23.2) | 73 (38.4) |
| Week 52, n (%) | | |
| Difference in response rate (95% CI) | 15.2 (5.9, 24.5) | |
| Nominal P-value | 0.001 | |
| Glucocorticoid reduction ≥25% | 59 (31.8) | 96 (50.5) |

| | |
|---|---|
| at Week 52, n (%) | |
| Difference in response rate (95% CI) | 18.7 (8.9, 28.4) |
| Nominal P-value | <0.001 |

CI, confidence interval;
IFNGS, interferon gene signature;
SLEDAI-2K, Systemic Lupus Erythematosus Disease Activity Index 2000.
Difference in response rates, 95% CIs, and nominal P-values were calculated using a stratified Cochran-Mantel-Haenszel approach.

### 8.4.5 PROS

FACIT-F, SF-36 PCS, and SF-36 MCS scores were similar for treatment groups and glucocorticoid responders and nonresponders at baseline (Table 8-9). Treatment with anifrolumab, compared with placebo, resulted in more patients with nominally significant improvement in SF-36 MCS scores (P=0.03), but not SF-36 PCS or FACIT-F (FIG. 6A-C). The glucocorticoid responders group had more patients with nominally significant improvements in all PROs (all P<0.001) compared with nonresponders (FIG. 62D-F).

TABLE 8-9
PRO scores at baseline in patients receiving
glucocorticoid ≥10 mg/day at baseline in
TULIP-1 and TULIP-2 (pooled data)

| | | | Gluco- | Gluco- |
|---|---|---|---|---|
| | | | corticoid | corticoid |
| | Placebo | Anifrolumab | responders^{a} | nonresponders^{a} |
| PRO | (n = 185) | (n = 190) | (n = 155) | (n = 220) |
| FACIT-F | | | | |
| N | 179 | 178 | 152 | 205 |
| Mean (SD) | 26.05 (12.06) | 26.87 (12.20) | 28.49 (12.18) | 24.96 (11.89) |
| SF-36 PCS | | | | |
| N | 177 | 179 | 153 | 203 |
| Mean (SD) | 36.96 (9.16) | 37.76 (9.29) | 39.51 (9.15) | 35.74 (8.97) |
| SF-36 MCS | | | | |
| N | 177 | 179 | 153 | 203 |
| Mean (SD) | 43.76 (11.69) | 44.09 (11.69) | 43.75 (11.39) | 44.06 (11.92) |

FACIT-F, Functional Assessment of Chronic Illness Therapy-Fatigue;
MCS, mental component score;
PCS, physical component score;
PRO, patient-reported outcome;
SD, standard deviation;
SF-36, Short Form 36 Health Survey.
^{a}Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

### 8.4.6 Association Between Glucocorticoid Responders and BICLA Responders

Of the patients in the anifrolumab group who achieved a BICLA response, 80.1% (72/89) had a sustained glucocorticoid reduction response (glucocorticoid responder) compared with 74.1% (43/58) of patients in the placebo group who achieved a BICLA response. Thus, anifrolumab treatment resulted in 37.8% (72/190) of patients achieving both a BICLA response and a glucocorticoid response at Week 52 compared with 23.2% (43/185) of placebo-treated patients.

A total of 46.8% (89/190) of patients treated with anifrolumab and receiving baseline glucocorticoids ≥10 mg/day achieved a BICLA response at Week 52 versus 31.4% (58/185) of patients who received placebo (FIG. 5, FIG. 7). In BICLA responders, a high proportion also achieved a sustained glucocorticoid taper response (80.9% [72/89] receiving anifrolumab and 74.1% [43/58] receiving placebo). Thus, with anifrolumab treatment, 37.8% (72/190) of patients achieved the combination of BICLA response and sustained glucocorticoid taper response at Week 52 compared with 23.3% (43/185) of patients who received placebo (difference 14.6%, 95% CI 5.3%, 23.9%, P=0.002) (FIG. 5, FIG. 7).

### 8.4.7 Changes in Clinical and Laboratory Values

### 8.4.7.1 Vital Signs

Mean baseline systolic and diastolic sitting blood pressure and heart rate were lower at Week 40, from when glucocorticoid dosage was required to be stable, with anifrolumab treatment compared with placebo (all nominal P<0.05); at Week 52, between-group treatment differences were not significantly different at Week 52 (Table 8-10). Similarly, at Week 40, glucocorticoid responders had lower systolic and diastolic sitting blood pressure compared with nonresponders (P=0.02 and P<0.001, respectively); differences in diastolic (P=0.01) but not systolic (P=0.38) sitting blood pressure were maintained at Week 52. Differences in heart rate between glucocorticoid responders and nonresponders did not reach nominal significance at Week 40 or Week 52 (Table 8-10). The use of supplementary blood pressure medications that started during treatment of patients in the anifrolumab group was 6.3% (12/190) and 18.4% (34/185) in the placebo group; 7.1% (11/155) of glucocorticoid responders and 15.9% (35/220) of nonresponders started new blood pressure medications during the study (Table 8-11).

TABLE 8-10
Change in blood pressure and heart rate at Week 40 and Week 52 in patients receiving
glucocorticoid ≥10 mg/day at baseline in TULIP-1 and TULIP-2 (pooled data)

| | | | |
|---|---|---|---|
| | | Glucocorticoid | Glucocorticoid |
| Placebo | Anifrolumab | responder^{a} | nonresponder^{a} |
| (n = 185) | (n = 190) | (n = 155) | (n = 220) |

Systolic, sitting blood pressure, mm Hg

| Baseline, mean (SD) | 119.27 | (13.835) | 118.72 | (13.134) | 119.33 | (12.84) | 118.75 | (13.92) |
|---|---|---|---|---|---|---|---|---|
| Week 40, change from | 1.18 | (1.143) | −2.16 | (1.115) | −2.26 | (1.159) | 0.93 | (1.106) |
| baseline, LS mean (SE) | | | | | | | | |
| Difference, LS mean (95% CI) | −3.34 (−6.08, −0.60) | | | | −3.19 (−5.94, −0.44) | | | |
| Nominal P-value | 0.017 | | | | 0.023 | | | |
| Week 52, change from | 1.47 | (1.168) | −0.17 | (1.142) | −0.05 | (1.160) | 1.22 | (1.155) |
| baseline, LS mean (SE) | | | | | | | | |

| | | |
|---|---|---|
| Difference, LS mean (95% CI) | −1.64 (−4.47, 1.19) | −1.27 (−4.10, 1.57) |
| Nominal P-value | 0.255 | 0.381 |

Diastolic, sitting blood pressure, mm Hg

| Baseline, mean (SD) | 74.68 | (9.644) | 74.56 | (8.947) | 75.20 | (9.17) | 74.21 | (9.37) |
|---|---|---|---|---|---|---|---|---|
| Week 40, change from | 0.49 | (0.788) | −1.72 | (0.768) | −2.42 | (0.785) | 0.99 | (0.760) |
| baseline, LS mean (SE) | | | | | | | | |
| Difference, LS mean (95% CI) | −2.22 (−4.11, −0.32) | | | | −3.41 (−5.29, −1.53) | | | |
| Nominal P-value | 0.022 | | | | <0.001 | | | |
| Week 52, change from | 0.90 | (0.846) | −0.45 | (0.826) | −1.17 | (0.832) | 1.55 | (0.832) |
| baseline, LS mean (SE) | | | | | | | | |

| | | |
|---|---|---|
| Difference, LS mean (95% CI) | −1.36 (−3.44, 0.72) | −2.72 (−4.80, −0.65) |
| Nominal P-value | 0.200 | 0.010 |

Heart rate, beats/min

| Baseline, mean (SD) | 75.50 | (10.938) | 75.71 | (11.678) | 74.17 | (10.99) | 76.62 | (11.44) |
|---|---|---|---|---|---|---|---|---|
| Week 40, change from | 0.97 | (0.829) | −1.18 | (0.811) | −1.08 | (0.842) | 0.71 | (0.805) |
| baseline, LS mean (SE) | | | | | | | | |
| Difference, LS mean (95% CI) | −2.16 (−4.14, −0.18) | | | | −1.78 (−3.78, 0.21) | | | |
| Nominal P-value | 0.033 | | | | 0.080 | | | |
| Week 52, change from | −0.47 | (0.819) | −2.15 | (0.803) | −1.62 | (0.816) | −1.11 | (0.813) |
| baseline, LS mean (SE) | | | | | | | | |

| | | |
|---|---|---|
| Difference, LS mean (95% CI) | −1.68 (−3.64, 0.28) | −0.50 (−2.48, 1.47) |
| Nominal P-value | 0.092 | 0.615 |

LS, least squares;
SD, standard deviation;
SE, standard error.
^{a}Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

TABLE 8-11
Blood pressure medications starting during treatment

| | | | Glucocorticoid | Glucocorticoid |
|---|---|---|---|---|
| | Placebo | Anifrolumab | responders^{a} | nonresponders^{a} |
| Medications, n (%) | (n = 185) | (n = 190) | (n = 155) | (n = 220) |
| Patients with any medication | 34 (18.4) | 12 (6.3) | 11 (7.1) | 35 (15.9) |
| Agents acting on | 21 (11.4) | 5 (2.6) | 6 (3.9) | 20 (9.1) |
| the renin-angiotensin system | | | | |
| Captopril | 1 (0.5) | 2 (1.1) | 2 (1.3) | 1 (0.5) |
| Enalapril | 4 (2.2) | 2 (1.1) | 1 (0.6) | 5 (2.3) |
| Lisinopril | 4 (2.2) | 0 | 1 (0.6) | 3 (1.4) |
| Ramipril | 2 (1.1) | 1 (0.5) | 1 (0.6) | 2 (0.9) |
| Valsartan | 2 (1.1) | 0 | 1 (0.6) | 1 (0.5) |
| Losartan | 5 (2.7) | 1 (0.5) | 0 | 6 (2.7) |
| Perindopril | 2 (1.1) | 0 | 0 | 2 (0.9) |
| Amlodipine; valsartan | 1 (0.5) | 0 | 0 | 1 (0.5) |
| Captopril; hydrochlorothiazide | 1 (0.5) | 0 | 0 | 1 (0.5) |
| Enalapril maleate; | 1 (0.5) | 0 | 0 | 1 (0.5) |
| hydrochlorothiazide | | | | |
| Olmesartan | 1 (0.5) | 0 | 0 | 1 (0.5) |
| Olmesartan medoxomil | 1 (0.5) | 0 | 0 | 1 (0.5) |
| Telmisartan | 1 (0.5) | 0 | 0 | 1 (0.5) |
| Beta blocking agents | 12 (6.5) | 2 (1.1) | 5 (3.2) | 9 (4.1) |
| Metoprolol | 2 (1.1) | 2 (1.1) | 1 (0.6) | 3 (1.4) |
| Bisoprolol | 3 (1.6) | 0 | 1 (0.6) | 2 (0.9) |
| Labetalol | 1 (0.5) | 0 | 1 (0.6) | 0 |
| Metoprolol succinate | 1 (0.5) | 0 | 1 (0.6) | 0 |
| Propranolol | 1 (0.5) | 0 | 1 (0.6) | 0 |
| Carvedilol | 2 (1.1) | 0 | 0 | 2 (0.9) |
| Metoprolol tartrate | 1 (0.5) | 0 | 0 | 1 (0.5) |
| Nebivolol hydrochloride | 1 (0.5) | 0 | 0 | 1 (0.5) |
| Calcium channel blockers | 9 (4.9) | 4 (2.1) | 3 (1.9) | 10 (4.5) |
| Amlodipine besylate; | 0 | 1 (0.5) | 1 (0.6) | 0 |
| indapamide | | | | |
| Nifedipine | 0 | 1 (0.5) | 1 (0.6) | 0 |
| Verapamil | 1 (0.5) | 0 | 1 (0.6) | 0 |
| Amlodipine | 6 (3.2) | 1 (0.5) | 0 | 7 (3.2) |
| Amlodipine besylate | 2 (1.1) | 0 | 0 | 2 (0.9) |
| Felodipine | 1 (0.5) | 0 | 0 | 1 (0.5) |
| Diuretics | 12 (6.5) | 4 (2.1) | 1 (0.6) | 15 (6.8) |
| Spironolactone | 3 (1.6) | 1 (0.5) | 1 (0.6) | 3 (1.4) |
| Furosemide | 5 (2.7) | 3 (1.6) | 0 | 8 (3.6) |
| Hydrochlorothiazide | 2 (1.1) | 1 (0.5) | 0 | 3 (1.4) |
| Chlortalidone | 2 (1.1) | 0 | 0 | 2 (0.9) |
| Amiloride | 1 (0.5) | 0 | 0 | 1 (0.5) |
| Hydrochlorothiazide; | 1 (0.5) | 0 | 0 | 1 (0.5) |
| triamterene | | | | |
| Indapamide | 1 (0.5) | 0 | 0 | 1 (0.5) |
| Antihypertensives | 3 (1.6) | 0 | 1 (0.6) | 2 (0.9) |
| Clonidine | 2 (1.1) | 0 | 1 (0.6) | 1 (0.5) |
| Moxonidine | 1 (0.5) | 0 | 0 | 1 (0.5) |

^{a}Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

### 8.4.7.1.1 Laboratory Values

LS mean changes in weight, BMI, fasting glucose, cholesterol, and laboratory blood values are provided in Table 8-12. LS mean changes at Week 24 and Week 52 were generally similar between treatment groups and between glucocorticoid responders and nonresponders. Of note, anifrolumab treatment and glucocorticoid response resulted in increases in weight and BMI from baseline at Week 24 and Week 52. Shift tables for BMI are shown in Table 8-13. Additionally, anifrolumab treatment resulted in increases in laboratory blood values (erythrocytes, leukocytes, lymphocytes, neutrophils, and platelets) compared with placebo, whereas patients in the placebo group had mean decreases from baseline or stable values.

TABLE 8-12
Changes from baseline in laboratory values at Week 24 and
Week 52 in patients receiving glucocorticoid ≥10 mg/day
at baseline in TULIP-1 and TULIP-2 (pooled data)

| | | | | | Glucocorticoid | | Glucocorticoid | |
|---|---|---|---|---|---|---|---|---|
| | Placebo | | Anifrolumab | | responders^{a} | | nonresponders^{a} | |
| | (n = 185) | | (n = 190) | | (n = 155) | | (n = 220) | |
| Weight, kg | | | | | | | | |
| Baseline, mean (SD) | 70.46 | (16.62) | 71.79 | (18.93) | 69.69 | (16.78) | 72.15 | (18.48) |
| Week 24, change from | 1.23 | (4.47) | 1.72 | (4.36) | 1.79 | (4.57) | 1.21 | (4.27) |
| baseline, LS mean (SE) | | | | | | | | |
| Week 52, change from | 0.84 | (6.27) | 2.25 | (5.59) | 1.70 | (6.55) | 1.44 | (5.29) |
| baseline, LS mean (SE) | | | | | | | | |
| BMI, kg/m² | | | | | | | | |
| Baseline, mean (SD) | 26.31 | (5.82) | 27.25 | (6.63) | 26.61 | (5.93) | 26.91 | (6.49) |
| Week 24, change from | 0.46 | (1.67) | 0.66 | (1.66) | 0.68 | (1.76) | 0.47 | (1.58) |
| baseline, LS mean (SE) | | | | | | | | |
| Week 52, change from | 0.30 | (2.41) | 0.85 | (2.11) | 0.64 | (2.54) | 0.53 | (1.96) |
| baseline, LS mean (SE) | | | | | | | | |
| Fasting glucose, mmol/L | | | | | | | | |
| Baseline, mean (SD) | 4.77 | (0.76) | 4.85 | (1.05) | 4.87 | (1.04) | 4.78 | (0.81) |
| Week 24, change from | 0.21 | (0.87) | 0.01 | (0.99) | 0.09 | (1.02) | 0.12 | (0.84) |
| baseline, LS mean (SE) | | | | | | | | |
| Week 52, change from | 0.14 | (1.05) | 0.02 | (0.96) | 0.10 | (1.07) | 0.04 | (0.92) |
| baseline, LS mean (SE) | | | | | | | | |
| Total cholesterol, mmol/L | | | | | | | | |
| Baseline, mean (SD) | 5.01 | (1.12) | 4.98 | (1.12) | 4.95 | (1.11) | 5.03 | (1.12) |
| Week 24, change from | 0.02 | (0.72) | 0.01 | (0.72) | −0.02 | (0.73) | 0.05 | (0.71) |
| baseline, LS mean (SE)) | | | | | | | | |
| Week 52, change from | −0.01 | (0.90) | −0.12 | (0.88) | −0.09 | (0.85) | −0.04 | (0.95) |
| baseline, LS mean (SE) | | | | | | | | |
| HDL, mmol/L | | | | | | | | |
| Baseline, mean (SD) | 1.48 | (0.42) | 1.52 | (0.49) | 1.52 | (0.45) | 1.48 | (0.46) |
| Week 24, change from | −0.04 | (0.33) | 0.10 | (0.34) | 0.07 | (0.37) | 0.00 | (0.31) |
| baseline, LS mean (SE) | | | | | | | | |
| Week 52, change from | −0.07 | (0.30) | 0.02 | (0.35) | −0.01 | (0.32) | −0.04 | (0.35) |
| baseline, LS mean (SE) | | | | | | | | |
| LDL, mmol/L | | | | | | | | |
| Baseline, mean (SD) | 2.83 | (0.92) | 2.73 | (0.88) | 2.71 | (0.87) | 2.83 | (0.92) |
| Week 24, change from | 0.03 | (0.56) | −0.06 | (0.62) | −0.03 | (0.58) | −0.01 | (0.60) |
| baseline, LS mean (SE) | | | | | | | | |
| Week 52, change from | 0.03 | (0.72) | −0.05 | (0.72) | −0.02 | (0.70) | −0.00 | (0.74) |
| baseline, LS mean (SE) | | | | | | | | |
| Triglycerides, mmol/L | | | | | | | | |
| Baseline, mean (SD) | 1.51 | (0.72) | 1.57 | (0.85) | 1.54 | (0.84) | 1.55 | (0.75) |
| Week 24, change from | 0.12 | (1.03) | −0.10 | (0.77) | −0.13 | (0.74) | 0.14 | (1.04) |
| baseline, LS mean (SE) | | | | | | | | |
| Week 52, change from | 0.07 | (0.67) | −0.20 | (0.73) | −0.13 | (0.77) | 0.01 | (0.63) |
| baseline, LS mean (SE) | | | | | | | | |
| Hematocrit | | | | | | | | |
| Baseline, mean (SD) | 0.38 | (0.05) | 0.38 | (0.05) | 0.39 | (0.04) | 0.38 | (0.05) |
| Week 24, change from | −0.00 | (0.04) | 0.01 | (0.03) | 0.0 | (0.03) | 0.0 | (0.03) |
| baseline, LS mean (SE) | | | | | | | | |
| Week 52, change from | −0.00 | (0.04) | 0.00 | (0.03) | −0.0 | (0.03) | 0.0 | (0.04) |
| baseline, LS mean (SE) | | | | | | | | |
| Erythrocytes, 10¹²/L | | | | | | | | |
| Baseline, mean (SD) | 4.27 | (0.50) | 4.23 | (0.51) | 4.31 | (0.49) | 4.21 | (0.51) |
| Week 24, change from | −0.00 | (0.33) | 0.11 | (0.34) | 0.05 | (0.35) | 0.06 | (0.33) |
| baseline, LS mean (SE) | | | | | | | | |
| Week 52, change from | 0.01 | (0.37) | 0.11 | (0.34) | 0.05 | (0.323) | 0.07 | (0.39) |
| baseline, LS mean (SE) | | | | | | | | |
| Leukocytes, 10⁹/L | | | | | | | | |
| Baseline, mean (SD) | 6.17 | (2.58) | 5.81 | (2.48) | 5.73 | (2.26) | 6.16 | (2.70) |
| Week 24, change from | −0.18 | (2.46) | 1.41 | (2.31) | 0.91 | (2.44) | 0.40 | (2.55) |
| baseline, LS mean (SE) | | | | | | | | |
| Week 52, change from | −0.13 | (2.45) | 1.05 | (2.41) | 0.57 | (2.32) | 0.39 | (2.67) |
| baseline, LS mean (SE) | | | | | | | | |
| Lymphocytes, 10⁹/L | | | | | | | | |
| Baseline, mean (SD) | 1.28 | (0.68) | 1.26 | (0.73) | 1.29 | (0.68) | 1.26 | (0.72) |
| Week 24, change from | −0.09 | (0.46) | 0.37 | (0.69) | 0.26 | (0.70) | 0.05 | (0.55) |
| baseline, LS mean (SE) | | | | | | | | |
| Week 52, change from | −0.03 | (0.58) | 0.36 | (0.74) | 0.24 | (0.65) | 0.11 | (0.74) |
| baseline, LS mean (SE) | | | | | | | | |
| Neutrophils, 10⁹/L | | | | | | | | |
| Baseline, mean (SD) | 4.45 | (2.28) | 4.10 | (2.04) | 3.98 | (1.85) | 4.47 | (2.34) |
| Week 24, change from | −0.09 | (2.39) | 0.93 | (2.12) | 0.58 | (2.23) | 0.31 | (2.37) |
| baseline, LS mean (SE) | | | | | | | | |
| Week 52, change from | −0.09 | (2.42) | 0.61 | (2.10) | 0.31 | (2.08) | 0.24 | (2.48) |
| baseline, LS mean (SE) | | | | | | | | |
| Platelets, 10⁹/L | | | | | | | | |
| Baseline, mean (SD) | 258.67 | (86.14) | 240.59 | (80.12) | 242.17 | (79.41) | 254.68 | (86.11) |
| Week 24, change from | −4.54 | (48.70) | 29.44 | (56.56) | 17.03 | (56.94) | 9.58 | (54.13) |
| baseline, LS mean (SE) | | | | | | | | |
| Week 52, change from | −0.96 | (53.19) | 28.55 | (59.94) | 19.22 | (56.99) | 9.49 | (60.08) |
| baseline, LS mean (SE) | | | | | | | | |

BMI, body mass index;
LS, least squares;
SD, standard deviation;
SE, standard error.
^{a}Glucocorticoid responder is defined as a glucocorticoid dosage reduction to ≤7.5 mg/day by Week 40 without a dosage increase between Week 40 and Week 52 for patients receiving ≥10 mg/day at baseline.

TABLE 8-13
BMI shift tables for changes from baseline to Week 24 and Week 52 in patients receiving
glucocorticoid ≥10 mg/day at baseline in TULIP-1 and TULIP-2 (pooled data)

| | Underweight: | Normal weight: | Overweight: | Obese: |
|---|---|---|---|---|
| | BMI <18.5, | 18.5 ≤ | 25 ≤ BMI < | BMI ≥30, |
| | n (%) | BMI < 25, n (%) | 30, n (%) | n (%) |
| Placebo (n = 185) | | | | |
| Week 24 | | | | |
| Underweight: | 4 (2.2) | 2 (1.1) | 0 | 0 |
| BMI <18.5, n (%) | | | | |
| Normal weight: | 5 (2.7) | 60 (32.4) | 5 (2.7) | 0 |
| 18.5 ≤ BMI < 25, n (%) | | | | |
| Overweight: | 0 | 7 (3.8) | 38 (20.5) | 1 (0.5) |
| 25 ≤ BMI < 30, n (%) | | | | |
| Obese: BMI ≥30, n (%) | 0 | 0 | 4 (2.2) | 37 (20.0) |
| Missing, n (%) | 0 | 9 (4.9) | 9 (4.9) | 4 (2.2) |
| Week 52 | | | | |
| Underweight: | 4 (2.2) | 1 (0.5) | 0 | 0 |
| BMI <18.5, n (%) | | | | |
| Normal weight: | 3 (1.6) | 54 (29.2) | 5 (2.7) | 0 |
| 18.5 ≤ BMI < 25, n (%) | | | | |
| Overweight: | 0 | 5 (2.7) | 27 (14.6) | 2 (1.1) |
| 25 ≤ BMI < 30, n (%) | | | | |
| Obese: BMI ≥30, n (%) | 0 | 0 | 5 (2.7) | 34 (18.4) |
| Missing, n (%) | 2 (1.1) | 18 (9.7) | 19 (10.3) | 6 (3.2) |
| Anifrolumab (n = 190) | | | | |
| Week 24 | | | | |
| Underweight: | 5 (2.6) | 0 | 0 | 0 |
| BMI <18.5, n (%) | | | | |
| Normal weight: | 0 | 63 (33.2) | 2 (1.1) | 0 |
| 18.5 ≤ BMI < 25, n (%) | | | | |
| Overweight: | 0 | 13 (6.8) | 29 (15.3) | 1 (0.5) |
| 25 ≤ BMI < 30, n (%) | | | | |
| Obese: BMI ≥30, n (%) | 0 | 0 | 6 (3.2) | 52 (27.4) |
| Missing, n (%) | 1 (0.5) | 6 (3.2) | 4 (2.1) | 8 (4.2) |
| Week 52 | | | | |
| Underweight: | 2 (1.1) | 0 | 0 | 0 |
| BMI <18.5, n (%) | | | | |
| Normal weight: | 1 (0.5) | 52 (27.4) | 3 (1.6) | 0 |
| 18.5 ≤ BMI < 25, n (%) | | | | |
| Overweight: | 0 | 16 (8.4) | 18 (9.5) | 4 (2.1) |
| 25 ≤ BMI < 30, n (%) | | | | |
| Obese: BMI ≥30, n (%) | 0 | 0 | 11 (5.8) | 45 (23.7) |
| Missing, n (%) | 3 (1.6) | 14 (7.4) | 9 (4.7) | 12 (6.3) |
| Glucocorticoid | | | | |
| responders^{a} | | | | |
| (n = 155) | | | | |
| Week 24 | | | | |
| Underweight: | 3 (1.9) | 1 (0.6) | 0 | 0 |
| BMI <18.5, n (%) | | | | |
| Normal weight: | 1 (0.6) | 63 (40.6) | 3 (1.9) | 0 |
| 18.5 ≤ BMI < 25, n (%) | | | | |
| Overweight: | 0 | 10 (6.5) | 28 (18.1) | 1 (0.6) |
| 25 ≤ BMI < 30, n (%) | | | | |
| Obese: BMI ≥30, n (%) | 0 | 0 | 9 (5.8) | 35 (22.6) |
| Missing, n (%) | 0 | 0 | 0 | 1 (0.6) |
| Week 52 | | | | |
| Underweight: | 1 (0.6) | 1 (0.6) | 0 | 0 |
| BMI <18.5, n (%) | | | | |
| Normal weight: | 2 (1.3) | 56 (36.1) | 5 (3.2) | 0 |
| 18.5 ≤ BMI < 25, n (%) | | | | |
| Overweight: | 0 | 14 (9.0) | 24 (15.5) | 4 (2.6) |
| 25 ≤ BMI < 30, n (%) | | | | |
| Obese: BMI ≥30, n (%) | 0 | 0 | 11 (7.1) | 31 (20.0) |
| Missing, n (%) | 1 (0.6) | 3(1.9) | 0 | 2 (1.3) |
| Glucocorticoid | | | | |
| nonresponders^{a} (n = 220) | | | | |
| Week 24 | | | | |
| Underweight: | 6 (2.7) | 1(0.5) | 0 | 0 |
| BMI <18.5, n (%) | | | | |
| Normal weight: | 4 (1.8) | 60 (27.3) | 4 (1.8) | 0 |
| 18.5 ≤ BMI < 25, n (%) | | | | |
| Overweight: | 0 | 10 (4.5) | 39 (17.7) | 1 (0.5) |
| 25 ≤ BMI < 30, n (%) | | | | |
| Obese: BMI ≥30, n (%) | 0 | 0 | 1 (0.5) | 54 (24.5) |
| Missing, n (%) | 1 (0.5) | 15 (6.8) | 13 (5.9) | 11 (5.0) |
| Week 52 | | | | |
| Underweight: | 5 (2.3) | 0 | 0 | 0 |
| BMI <18.5, n (%) | | | | |
| Normal weight: | 2 (0.9) | 50 (22.7) | 3 (1.4) | 0 |
| 18.5 ≤ BMI < 25, n (%) | | | | |
| Overweight: | 0 | 7 (3.2) | 21 (9.5) | 2 (0.9) |
| 25 ≤ BMI < 30, n (%) | | | | |
| Obese: BMI ≥30, n (%) | 0 | 0 | 5 (2.3) | 48 (21.8) |
| Missing, n (%) | 4 (1.8) | 29 (13.2) | 28 (12.7) | 16 (7.3) |

BMI, body mass index
^{a}Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

### 8.4.8 Safety

The incidence of serious AEs was 21.1% (40/190) in the anifrolumab group and 25.9% (48/185) in the placebo group; 16.8% (26/155) of glucocorticoid responders and 28.2% (62/220) of nonresponders reported serious AEs (Table 8-14). Cardiovascular AEs were reported in 10.0% and 13.5% of patients in the anifrolumab and placebo groups, respectively, and in 11.4% and 12.3% of glucocorticoid responders and nonresponders, respectively (Table 8-15). Hypertension was the most common cardiovascular AE reported for all groups.

TABLE 8-14
SAEs during treatment in patients receiving glucocorticoid ≥10 mg/
day at baseline in TULIP-1 and TULIP-2 (pooled data)

| | | | Glucocorticoid | Glucocorticoid |
|---|---|---|---|---|
| | Placebo | Anifrolumab | responders^{a} | nonresponders^{a} |
| SAEs, n (%) | (n = 185) | (n = 190) | (n = 155) | (n = 220) |
| Patients with any SAE | 48 (25.9) | 40 (21.1) | 26 (16.8) | 62 (28.2) |
| Systemic lupus | 11 (5.9) | 4 (2.1) | 4 (2.6) | 11 (5.0) |
| erythematosus | | | | |
| Pneumonia | 8 (4.3) | 6 (3.2) | 2 (1.3) | 12 (5.5) |
| Influenza | 1 (0.5) | 1 (0.5) | 2 (1.3) | 0 |
| Herpes zoster | 2 (1.1) | 1 (0.5) | 1 (0.6) | 2 (0.9) |
| Coronary artery disease | 0 | 2 (1.1) | 1 (0.6) | 1 (0.5) |
| Lupus nephritis | 2 (1.1) | 0 | 1 (0.6) | 1 (0.5) |
| Osteonecrosis | 1 (0.5) | 1 (0.5) | 1 (0.6) | 1 (0.5) |
| Pulmonary embolism | 1 (0.5) | 1 (0.5) | 1 (0.6) | 1 (0.5) |
| Abortion spontaneous | 0 | 1 (0.5) | 1 (0.6) | 0 |
| Acute coronary syndrome | 0 | 1 (0.5) | 1 (0.6) | 0 |
| Arthritis | 0 | 1 (0.5) | 1 (0.6) | 0 |
| Cervical dysplasia | 0 | 1 (0.5) | 1 (0.6) | 0 |
| Chest pain | 0 | 1 (0.5) | 1 (0.6) | 0 |
| Cholelithiasis | 0 | 1 (0.5) | 1 (0.6) | 0 |
| Dyspnea | 0 | 1 (0.5) | 1 (0.6) | 0 |
| Erysipelas | 1 (0.5) | 0 | 1 (0.6) | 0 |
| Facial bones fracture | 0 | 1 (0.5) | 1 (0.6) | 0 |
| Herpes zoster | 0 | 1 (0.5) | 1 (0.6) | 0 |
| disseminated | | | | |
| Herpes zoster meningitis | 1 (0.5) | 0 | 1 (0.6) | 0 |
| Humerus fracture | 0 | 1 (0.5) | 1 (0.6) | 0 |
| Hypersensitivity | 0 | 1 (0.5) | 1 (0.6) | 0 |
| Incarcerated hernia | 1 (0.5) | 0 | 1 (0.6) | 0 |
| Esophageal stenosis | 1 (0.5) | 0 | 1 (0.6) | 0 |
| Pleural effusion | 0 | 1 (0.5) | 1 (0.6) | 0 |
| Pneumonia staphylococcal | 1 (0.5) | 0 | 1 (0.6) | 0 |
| Post herpetic neuralgia | 0 | 1 (0.5) | 1 (0.6) | 0 |
| Renal impairment | 0 | 1 (0.5) | 1 (0.6) | 0 |
| Tendon rupture | 1 (0.5) | 0 | 1 (0.6) | 0 |
| Tenosynovitis | 0 | 1 (0.5) | 1 (0.6) | 0 |
| Upper limb fracture | 0 | 1 (0.5) | 1 (0.6) | 0 |
| Urosepsis | 1 (0.5) | 0 | 1 (0.6) | 0 |
| Urticaria | 1 (0.5) | 0 | 1 (0.6) | 0 |
| Uterine prolapse | 1 (0.5) | 0 | 1 (0.6) | 0 |
| Ventricular arrhythmia | 1 (0.5) | 0 | 1 (0.6) | 0 |
| Acute kidney injury | 1 (0.5) | 2 (1.1) | 0 | 3 (1.4) |
| Pyelonephritis | 0 | 3 (1.6) | 0 | 3 (1.4) |
| UNCODED | | | 0 | 2 (0.9) |
| Acute respiratory failure | 0 | 2 (1.1) | 0 | 2 (0.9) |
| Asthma | 1 (0.5) | 1 (0.5) | 0 | 2 (0.9) |
| Bronchitis | 1 (0.5) | 1 (0.5) | 0 | 2 (0.9) |
| Syncope | 1 (0.5) | 1 (0.5) | 0 | 2 (0.9) |
| Urinary tract infection | 2 (1.1) | 0 | 0 | 2 (0.9) |
| Abscess | 1 (0.5) | 0 | 0 | 1 (0.5) |
| Abscess limb | 0 | 1 (0.5) | 0 | 1 (0.5) |
| Anemia | 1 (0.5) | 0 | 0 | 1 (0.5) |
| Atrial fibrillation | 1 (0.5) | 0 | 0 | 1 (0.5) |
| B-cell lymphoma | 0 | 1 (0.5) | 0 | 1 (0.5) |
| Cardiac failure | 1 (0.5) | 0 | 0 | 1 (0.5) |
| Cellulitis | 0 | 1 (0.5) | 0 | 1 (0.5) |
| Chronic kidney disease | 1 (0.5) | 0 | 0 | 1 (0.5) |
| Colitis | 0 | 1 (0.5) | 0 | 1 (0.5) |
| Conversion disorder | 0 | 1 (0.5) | 0 | 1 (0.5) |
| Dengue fever | 0 | 1 (0.5) | 0 | 1 (0.5) |
| Endometrial hypertrophy | 1 (0.5) | 0 | 0 | 1 (0.5) |
| Fall | 0 | 1 (0.5) | 0 | 1 (0.5) |
| Gastroenteritis | 1 (0.5) | 0 | 0 | 1 (0.5) |
| Gastroesophageal reflux | 0 | 1 (0.5) | 0 | 1 (0.5) |
| disease | | | | |
| Genital herpes | 0 | 1 (0.5) | 0 | 1 (0.5) |
| Hemangioma of liver | 1 (0.5) | 0 | 0 | 1 (0.5) |
| Hemorrhoidal hemorrhage | 0 | 1 (0.5) | 0 | 1 (0.5) |
| Hydronephrosis | 1 (0.5) | 0 | 0 | 1 (0.5) |
| Hypercalcemia | 0 | 1 (0.5) | 0 | 1 (0.5) |
| Hypoesthesia | 0 | 1 (0.5) | 0 | 1 (0.5) |
| Hypotension | 1 (0.5) | 0 | 0 | 1 (0.5) |
| Iron deficiency anemia | 1 (0.5) | 0 | 0 | 1 (0.5) |
| Large intestine infection | 1 (0.5) | 0 | 0 | 1 (0.5) |
| Malignant hypertension | 0 | 1 (0.5) | 0 | 1 (0.5) |
| Meningitis viral | 0 | 1 (0.5) | 0 | 1 (0.5) |
| Musculoskeletal chest | 0 | 1 (0.5) | 0 | 1 (0.5) |
| pain | | | | |
| Myasthenia gravis | 0 | 1 (0.5) | 0 | 1 (0.5) |
| Myocardial infarction | 0 | 1 (0.5) | 0 | 1 (0.5) |
| Nephrolithiasis | 0 | 1 (0.5) | 0 | 1 (0.5) |
| Neutropenia | 0 | 1 (0.5) | 0 | 1 (0.5) |
| Noncardiac chest pain | | | 0 | 1 (0.5) |
| Pain | 1 (0.5) | 0 | 0 | 1 (0.5) |
| Pelvic inflammatory | 0 | 1 (0.5) | 0 | 1 (0.5) |
| disease | | | | |
| Peritonsillar abscess | 0 | 1 (0.5) | 0 | 1 (0.5) |
| Pneumonia bacterial | 0 | 1 (0.5) | 0 | 1 (0.5) |
| Post procedural | 0 | 1 (0.5) | 0 | 1 (0.5) |
| complication | | | | |
| Postoperative wound | 1 (0.5) | 0 | 0 | 1 (0.5) |
| infection | | | | |
| Pulmonary alveolar | 1 (0.5) | 0 | 0 | 1 (0.5) |
| hemorrhage | | | | |
| Pyelonephritis acute | 0 | 1 (0.5) | 0 | 1 (0.5) |
| Renal failure | 1 (0.5) | 0 | 0 | 1 (0.5) |
| Respiratory failure | 1 (0.5) | 0 | 0 | 1 (0.5) |
| Sepsis | 1 (0.5) | 0 | 0 | 1 (0.5) |
| Septic shock | 1 (0.5) | 0 | 0 | 1 (0.5) |
| Spinal compression | 0 | 1 (0.5) | 0 | 1 (0.5) |
| fracture | | | | |
| Spinal stenosis | 0 | 1 (0.5) | 0 | 1 (0.5) |
| Streptococcal urinary tract | 0 | 1 (0.5) | 0 | 1 (0.5) |
| infection | | | | |
| Supraventricular | 1 (0.5) | 0 | 0 | 1 (0.5) |
| tachycardia | | | | |
| Swelling face | 1 (0.5) | 0 | 0 | 1 (0.5) |
| Synovial cyst | 0 | 1 (0.5) | 0 | 1 (0.5) |
| Traumatic fracture | 0 | 1 (0.5) | 0 | 1 (0.5) |
| Ulcerative keratitis | 1 (0.5) | 0 | 0 | 1 (0.5) |
| Uterine cancer | 1 (0.5) | 0 | 0 | 1 (0.5) |
| Uterovaginal prolapse | 1 (0.5) | 0 | 0 | 1 (0.5) |
| Venous thrombosis limb | 1 (0.5) | 0 | 0 | 1 (0.5) |
| Wound infection | 1 (0.5) | 0 | 0 | 1 (0.5) |
| staphylococcal | | | | |

SAE, serious adverse event
^{a}Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

TABLE 8-15
Cardiovascular AEs during treatment in patients
receiving glucocorticoid ≥10 mg/day at
baseline in TULIP-1 and TULIP-2 (pooled data)

| | | | Glucocorticoid | Glucocorticoid |
|---|---|---|---|---|
| | Placebo | Anifrolumab | responders^{a} | nonresponders^{a} |
| AEs, n (%) | (n = 185) | (n = 190) | (n = 155) | (n = 220) |
| Patients with any AE | 25 (13.5) | 19 (10.0) | 19 (12.3) | 25 (11.4) |
| Hypertension | 11 (5.9) | 4 (2.1) | 5 (3.2) | 10 (4.5) |
| Essential hypertension | 0 | 1 (0.5) | 1 (0.6) | 0 |
| Hypotension | 1 (0.5) | 0 | 0 | 1 (0.5) |
| Thrombosis | 1 (0.5) | 0 | 0 | 1 (0.5) |
| Vasodilatation | 1 (0.5) | 0 | 0 | 1 (0.5) |
| Venous thrombosis limb | 1 (0.5) | 0 | 0 | 1 (0.5) |
| Palpitations | 3 (1.6) | 0 | 1 (0.6) | 2 (0.9) |
| Coronary artery disease | 0 | 2 (1.1) | 1 (0.6) | 1 (0.5) |
| Sinus bradycardia | 1 (0.5) | 1 (0.5) | 1 (0.6) | 1 (0.5) |
| Acute coronary syndrome | 0 | 1 (0.5) | 1 (0.6) | 0 |
| Bradycardia | 1 (0.5) | 0 | 1 (0.6) | 0 |
| Bundle branch block right | 0 | 1 (0.5) | 1 (0.6) | 0 |
| Left ventricular dilatation | 0 | 1 (0.5) | 1 (0.6) | 0 |
| Ventricular arrhythmia | 1 (0.5) | 0 | 1 (0.6) | 0 |
| Supraventricular | 2 (1.1) | 0 | 0 | 2 (0.9) |
| tachycardia | | | | |
| Tachycardia | 0 | 2 (1.1) | 0 | 2 (0.9) |
| Atrial fibrillation | 1 (0.5) | 0 | 0 | 1 (0.5) |
| Cardiac failure | 1 (0.5) | 0 | 0 | 1 (0.5) |
| Cardiomyopathy | 1 (0.5) | 0 | 0 | 1 (0.5) |
| Myocardial infarction | 0 | 1 (0.5) | 0 | 1 (0.5) |
| Syncope | 1 (0.5) | 3 (1.6) | 0 | 4 (1.8) |

AE, adverse event
^{a}Glucocorticoid responder defined as a glucocorticoid dosage reduction to ≤7.5 mg/day by Week 40 without a dosage increase between Week 40 and Week 52 in patients with a baseline glucocorticoid dosage ≥10 mg/day.

### 8.5 Discussion

Controlling disease activity and avoiding drug toxicity from glucocorticoid use, are two of the most important treatment goals highlighted in SLE disease management guidelines. In this analysis of pooled data from the TULIP-1 and TULIP-2 trials of anifrolumab in patients with moderate to severe SLE, the inventors assessed the downstream effects of a sustained glucocorticoid taper regardless of treatment assignment. Sustained glucocorticoid tapering was associated with a 44% reduction in the mean cumulative glucocorticoid dose used over 52 weeks. Patients with sustained glucocorticoid tapering were more likely to have meaningful improvements in fatigue, physical and mental health, and reduced blood pressures compared with glucocorticoid nonresponders. A sustained taper was also associated with fewer SAEs, including infections.

Anifrolumab treatment facilitated more glucocorticoid tapering compared with placebo, and anifrolumab-treated patients were more likely to achieve the combination of sustained glucocorticoid taper and reduced disease activity.

In this post hoc analysis of pooled data from the TULIP-1 and TULIP-2 trials, among patients receiving baseline glucocorticoid doses of ≥10 mg/day, those who received anifrolumab were more likely to have reductions in glucocorticoid dose than were those receiving placebo. While facilitating glucocorticoid taper, anifrolumab treatment also had a beneficial effect on disease activity, blood pressure, laboratory blood values, and health-related quality of life. Regardless of treatment group, patients who were able to taper glucocorticoids had improvements similar to or greater than those observed with anifrolumab treatment.

In patients with SLE, persistent disease activity and protracted glucocorticoid treatment is a major predictor of organ damage. Thus, reduction of glucocorticoid use while improving disease activity is one of the most important treatment goals for the management of SLE for both clinicians and patients. However, complete and steroid-free clinical remission are hard to reach and maintain for some patients, particularly those receiving prolonged glucocorticoid therapy⁴⁷. Nevertheless, reducing glucocorticoid exposure is beneficial and has been reported to limit the negative adverse effects of glucocorticoids, regardless of whether the patient reaches a low dosage (≤7.5 mg/day), as each 1 mg/day reduction in mean prednisone dosage is estimated to be associated with an estimated 3%-6% reduced risk of future organ damage. In our analysis, in addition to sustained dosage reductions, anifrolumab-treated patients had reductions in daily glucocorticoid dose, reductions in cumulative dose over 52 weeks, greater threshold reductions, and fewer dosage increases than did patients receiving placebo, all of which could provide long-term health benefit for patients with SLE. A sustained glucocorticoid taper was associated with improvements in PROs, including less fatigue and improved physical and mental health. The mechanisms behind these improvements are likely, in part, to be directly related to reduced glucocorticoid dosage as sleep disturbance, mood disorders, and catabolic effects on muscle are all recognised adverse effects of higher glucocorticoid dosages.

Glucocorticoid use is reported to be a risk factor of coronary heart disease in patients with SLE, independent of SLE disease activity. Many reports have associated prednisone dose with increases in total serum cholesterol, blood pressure, blood glucose, triglycerides, and body weight⁴⁸. The inventors examined treatment differences in several areas of cardiovascular health, including systolic and diastolic blood pressure, heart rate, new blood pressure medications during treatment, and cardiovascular AEs. Significant lowering of systolic and diastolic blood pressure was noted at Week 40 in patients treated with anifrolumab. The use of supplementary blood pressure medications by those randomized to anifrolumab consistently exceeded that by patients randomized to placebo, which may have confounded the treatment differences at Week 52. There was no difference in the proportion of glucocorticoid responders and nonresponders starting new blood pressure medications during the study. Consistent with reports of hypertension in up to 74% of patients with SLE, hypertension was the most common cardiovascular AE reported in 2%-6% of patients across treatment and responder groups.

The inventors found that glucocorticoid tapering was associated with measured reductions in systolic and diastolic blood pressure. In addition, fewer patients who tapered glucocorticoids started a new antihypertensive medication during the trial. Since this intervention would tend to minimise the absolute differences in observed blood pressure, lower blood pressure is a real benefit of glucocorticoid tapering in this population. These changes in blood pressure may contribute to a lower long-term risk of future cardiovascular disease in this population.

Unexpectedly, mean changes in weight and BMI showed modest increases in weight for patients treated with anifrolumab and patients classified as glucocorticoid responders. This result may be because of improved disease activity in patients during the TULIP trials, such that weight gain was in response to improved health status.

In conclusion, in pooled data from patients with moderate to severe SLE in the TULIP-1 and TULIP-2 trials, anifrolumab treatment enabled the reduction of oral glucocorticoid therapy while concurrently improving overall SLE disease activity. These results support the potential for anifrolumab to reduce cumulative glucocorticoid dosage and the consequent glucocorticoid-associated risk of adverse effects, a goal of long-term SLE treatment.

### 9 Example 4: Novel Stringent Outcome Measures Applied to the Phase 2 and 3 Anifrolumab Trials

### 9.1 Background

Treatment of patients with systemic lupus erythematosus (SLE) should aim to lower disease activity and prevent flares, maintained with the lowest possible dose of glucocorticoids (GC). The British Isles Lupus Assessment Group (BILAG)-based Composite Lupus Assessment (BICLA) is an assessment of global disease activity that is frequently evaluated in SLE clinical trials. A BICLA response requires improvement in all domains affected at baseline, assessed by BILAG-2004, no worsening of other BILAG-2004 domains, and no worsening vs baseline of both SLE Disease Activity Index 2000 (SLEDAI-2K) and Physician's Global Assessment (PGA).

Patients with systemic lupus erythematosus (SLE) who received anifrolumab, a type I interferon receptor antibody, had greater BILAG-based Composite Lupus Assessment (BICLA) response rates vs placebo at Week (W) 52 in the phase 2 MUSE and the phase 3 TULIP-1 and TULIP-2 trials. Patients receiving anifrolumab also had fewer flares, and more patients were able to taper glucocorticoids (GC) vs placebo.

### 9.2 Objectives

To evaluate anifrolumab treatment response vs placebo in patients with SLE from TULIP-2, TULIP-1, and MUSE using more stringent BICLA definitions, as well as a novel endpoint that requires dual BICLA and SLE Responder Index (SRI [4]) responses.

### 9.3 Methods

MUSE, TULIP-1, and TULIP-2 were randomized, placebo-controlled, 52-week trials of intravenous anifrolumab (every 4 weeks for 48 weeks) in patients with moderate to severe SLE despite standard therapy For patients receiving GC ≥10 mg/day at baseline, taper to ≤7.5 mg/day was considered sustained if achieved by W40 and sustained through W52. For patients receiving GC <10 mg/day at baseline, GC taper was sustained if the W40 dose was less than or equal to the baseline dose, with no increase from W40-W52. In this post hoc analysis, response rates for 5 novel endpoints were compared between anifrolumab 300 mg vs placebo groups for patients who: 1) met both BICLA and SRI (4) response criteria; 2) attained a W52 BICLA response with sustained GC taper; 3) attained a W52 BICLA response and no flares after W12 (flare defined as ≥1 new BILAG-2004 A or ≥2 new BILAG-2004 B scores vs the prior visit); 4) attained a W52 BICLA response with sustained GC taper and no flares after W12; and 5) attained a modified BICLA (mBICLA, crBICLA) response at W52 that required complete resolution of all baseline BILAG-2004 activity (all baseline A/B scores to D; no worsening of C or D scores).

### 9.3.1 Novel Stringent Outcomes Measures

In this post hoc analysis, response rates for 5 novel endpoints were compared between anifrolumab 300 mg vs placebo groups for patients who: 1) met both BICLA and SRI (4) response criteria; 2) attained a W52 BICLA response with sustained GC taper; 3) attained a W52 BICLA response and no flares after W12 (flare defined as ≥1 new BILAG-2004 A or ≥2 new BILAG-2004 B scores vs the prior visit); 4) attained a W52 BICLA response with sustained GC taper and no flares after W12; and 5) attained a modified BICLA (crBICLA) response at W52 that required complete resolution of all baseline BILAG-2004 activity (all baseline A/B scores to D; no worsening of C or D scores) (Table 9-1).

TABLE 9-1
Novel stringent outcome measures applied to data
from the TULIP-2, TULIP-1, and MUSE trials

| Endpoint | Definition |
|---|---|
| 1) BICLA response at Week 52 with | BICLA response^{a} + GC taper achieved by Week 40 and |
| sustained GC taper | sustained through Week 52 (GC taper defined as a |
| | dosage of ≤7.5 mg/day in patients receiving ≥10 mg/day |
| | prednisone or equivalent at baseline) |
| 2) BICLA response at Week 52 and no | BICLA response^{a} + no flares after Week 12, where flare |
| flares | was defined as ≥1 new BILAG-2004 A or ≥2 new |
| | BILAG-2004 B scores vs the prior visit |
| 3) BICLA response at Week 52 with | BICLA response^{a} + GC taper achieved by Week 40 and |
| sustained GC taper and no flares | sustained through Week 52 (as above) + no flares after |
| | Week 12 (as above) |
| 4) crBICLA (BICLA requiring complete | BICLA response^{a} modified to require resolution of all |
| resolution) response at Week 52 and by | baseline BILAG-2004 A/B scores to D and no worsening |
| treatment visit | of C or D scores |
| 5) Dual BICLA and SRI(4) response at | Met both BICLA response^{a} and SRI(4) response^{b} criteria |
| Week 52 | (meaning patients had improvement in all organ systems |
| | with involvement at baseline and complete resolution of |
| | enough disease manifestations to achieve a ≥4-point |
| | improvement in SLEDAI-2K score) |

BICLA, British Isles Lupus Assessment Group-based Composite Lupus Assessment;
BILAG, British Isles Lupus Assessment Group;
crBICLA, complete-resolution BICLA;
GC, glucocorticoid;
PGA, Physician's Global Assessment;
SLEDAI-2K, Systemic Lupus Erythematosus Disease Activity Index 2000;
SRI(4), Systemic Lupus Erythematosus Responder Index of ≥4.
^{a}BICLA response² defined as all of the following: reduction of all baseline BILAG-2004 A and B domain scores to B/C/D and C/D, respectively, and no worsening in other BILAG-2004 organ systems; no increase in SLEDAI-2K score (from baseline); no increase in PGA score (≥0.3 points from baseline); no discontinuation of investigational product (IP); and no use of restricted medications beyond protocol-allowed thresholds.
^{b}SRI(4) response⁷ defined as all of the following: ≥4-point reduction in SLEDAI-2K; <1 new BILAG-004 A or <2 new BILAG-2004 B scores; <0.3-point increase in PGA from baseline; no use of restricted medications beyond protocol-allowed thresholds; and no discontinuation of IP.

### 9.3.2 Statistical Analysis

Response rates, treatment differences, 95% confidence intervals (Cls), odds ratios, standard errors, and nominal P values were calculated using a stratified Cochran-Mantel-Haenszel approach6 (stratification factors: SLEDAI-2K score at screening, Day 1 GC dosage, and interferon gene signature [IFNGS] status at screening).

### 9.4 Results

Evaluated patients received anifrolumab 300 mg (MUSE, n=99; TULIP-1 and TULIP-2, n=180) or placebo (MUSE, n=102; TULIP-1, n=184; TULIP-2, n=182). Demographics and baseline disease characteristics were generally balanced (Table 9-2).

Response rate differences favouring anifrolumab 300 mg over placebo were observed for all 5 stringent BICLA endpoints across MUSE, TULIP-1, and TULIP-2 (FIG. 8). More patients met response criteria for both BICLA and SRI (4) at W52 with anifrolumab vs placebo (treatment difference, 14.3%-28.6%; nominal P≤0.004). A greater proportion of patients had BICLA responses at W52 with sustained GC taper with anifrolumab vs placebo. More patients had BICLA responses at W52 with no flares after W12 with anifrolumab vs placebo. More patients had BICLA responses at W52 with both sustained GC taper and no flares after W12 with anifrolumab vs placebo (treatment difference, 15.3%-19.3%; nominal P≤0.006). More patients attained crBICLA responses (requiring complete resolution of baseline disease activity) at W52 with anifrolumab vs placebo (treatment difference, 11.1%-14.1%; nominal P≤0.017).

Odds ratios favouring anifrolumab 300 mg over placebo were observed for all 5 endpoints at Week 52 (FIG. 8)
BICLA response+sustained GC taper, range: 1.72-3.97
BICLA response+no flares after Week 12, range: 2.30-3.47
BICLA response+no flares after Week 12+sustained GC taper, range: 2.65-4.16
Complete-resolution BICLA (crBICLA) response (requiring complete resolution of BILAG-2004 A/B scores), range: 2.45-2.74
BICLA+SRI (4) response, range: 1.89-3.76

Positive treatment differences favouring anifrolumab over placebo for crBICLA response were observed from approximately Week 32 (Week 28 in TULIP-1) and sustained through Week 52 in TULIP-2, TULIP-1, and MUSE (FIG. 9)

TABLE 9-2
Patient Demographics and Baseline Clinical Characteristics

| | TULiP-2 | | | | TULiP-1 | | | | MUSE | | | |
|---|---|---|---|---|---|---|---|---|---|---|---|---|
| | | | Anifrolumab | | | | Anifrolumab | | | | Anifrolumab | |
| | Placebo | | 300 mg | | Placebo | | 300 mg | | Placebo | | 300 mg | |
| | (n = 182) | | (n = 180) | | (n = 184) | | (n = 180) | | (n = 102) | | (n = 99) | |
| Age, mean (SD), years | 41.1 | (11.5) | 43.1 | (12.0) | 41.0 | (12.3) | 42.0 | (12.0) | 39.3 | (12.9) | 39.1 | (11.9) |
| Female, n (%) | 170 | (93.4) | 168 | (93.3) | 171 | (92.9) | 165 | (91.7) | 93 | (91.2) | 93 | (93.9) |
| Race, n (%) | | | | | | | | | | | | |
| White | 107 | (58.8) | 110 | (61.1) | 137 | (74.5) | 125 | (69.4) | 41 | (40.2) | 35 | (35.4) |
| Black/African American | 25 | (13.7) | 17 | (9.4) | 23 | (12.5) | 29 | (16.1) | 12 | (11.8) | 19 | (19.2) |
| Asian | 30 | (16.5) | 30 | (16.7) | 5 | (2.7) | 11 | (6.1) | 13 | (12.7) | 3 | (3.0) |
| Other | 12 | (6.6) | 15 | (8.3) | 19 | (10.3) | 15 | (8.3) | 36 | (35.3) | 38 | (38.4) |
| BILAG-2004, n (%) | | | | | | | | | | | | |
| ≥1 A | 95 | (52.2) | 81 | (45.0) | 84 | (45.7) | 93 | (51.7) | 49 | (48.0) | 52 | (52.5) |
| 0 As, ≥Bs | 78 | (42.9) | 91 | (50.6) | 84 | (45.7) | 79 | (43.9) | 48 | (47.1) | 41 | (41.4) |
| SLEDAI-2K global | 11.5 | (3.9) | 11.4 | (3.6) | 11.5 | (3.5) | 11.3 | (4.0) | 11.1 | (4.4) | 10.7 | (3.7) |
| score, mean (SD) | | | | | | | | | | | | |
| PGA score, mean (SD) | 1.8 | (0.4) | 1.7 | (0.4) | 1.8 | (0.4) | 1.9 | (0.4) | 1.8 | (0.4) | 1.9 | (0.4) |
| GC^{a}, n (%) | 151 | (83.0) | 141 | (78.3) | 153 | (83.2) | 150 | (83.3) | 88 | (86.3) | 79 | (79.8) |
| ≥10 mg/day | 83 | (45.6) | 87 | (48.3) | 102 | (55.4) | 103 | (57.2) | 64 | (62.7) | 55 | (55.6) |

BILAG, British Isles Lupus Assessment Group; GC, glucocorticoid; PGA, Physician's Global Assessment; SD, standard deviation; SLEDAI-2K, Systemic Lupus Erythematosus Disease Activity Index 2000.
^{a}Prednisone or equivalent.

### 9.5 Conclusions

In phase 2 and 3 trials in patients with SLE, anifrolumab treatment was consistently associated with improved disease control vs placebo using 5 novel, stringent BICLA based endpoint definitions, including BICLA response with sustained GC taper and no flares, BICLA response requiring complete resolution of baseline disease activity, and dual BICLA and SRI (4) responses. crBICLA response, requiring complete resolution of all baseline BILAG-2004 A/B scores, was sustained from as early as Week 28 through Week 52. These results support the ability of anifrolumab to reduce global disease activity, control flares, and minimize GC use, key treatment goals in patients with SLE.

### 10 Example 5: Injection Device

Anifrolumab is administered by an injection device [1] [9] such as a prefilled syringe (PFS) (FIG. 10A) or an autoinjector (AI) (FIG. 10B).

### 10.1 Autoinjector

Anifrolumab may be administered by an autoinjector [1]. The autoinjector is shown in exploded view (FIG. 11A) and in an assembled form (FIG. 11B). A label [4] is wrapped around and attached to the autoinjector [1] (FIG. 11C). The autoinjector has an autoinjector housing [3], cap and cap remover [2] and drive unit [5]. The liquid anifrolumab formulation unit dose [6] is contained in the autoinjector housing [3]. The unit dose [6] can be viewed through the viewing window [7].

### 10.2 Accessorized Pre-Frilled Syringe

Anifrolumab may be administered by accessorized pre-filled syringe (APFS) [8]. The APFS [8] includes the unit dose of anifrolumab [6] contained in a primary container [9] shown in an assembled state in FIG. 12A and in an exploded view in FIG. 12B. The primary container [9] has a plunger stopper [16]. The primary container has a nominal fill volume of 0.8 ml but may contain slightly more than 0.8 ml. The remainder of the space in the primary container [9] is taken up by an air bubble [18]. The air bubble may have a size of 3-5 mm, optionally, 4 mm. The primary container [9] has a defined stopper position [19].

The accessorized pre-filled syringe (APFS) primary container [9] is provided in a PFS assembly [8] including a needle guard [12], a finger flange and a plunger rod (FIG. 12C, FIG. 12D). A label is provided with the primary container [9] in the PFS assembly [8]. The label is wrapped around the syringe [9] in the label placement position [15].

### 10.3 Packaging

The injection device [1] [8] is provided in a kit (FIG. 13). A label [4] is provided with the APFS or autoinjector in the packaging. The label includes instruction for the use of the injection device [1], [8]. The packaging includes a tamper seal.

### REFERENCES

All publications mentioned in the specification are herein incorporated by reference.
(1) Pons-Estel, G. J.; Alarcón, G. S.; Scofield, L.; Reinlib, L.; Cooper, G. S. Understanding the Epidemiology and Progression of Systemic Lupus Erythematosus. Semin Arthritis Rheum 2010, 39 (4), 257-268. https://doi.org/10.1016/j.semarthrit.2008.10.007.
(2) McCauliffe, D. P. Cutaneous Lupus Erythematosus. Semin Cutan Med Surg 2001, 20 (1), 14-26. https://doi.org/10.1053/sder.2001.23091.
(3) Uva, L.; Miguel, D.; Pinheiro, C.; Freitas, J. P.; Marques Gomes, M.; Filipe, P. Cutaneous Manifestations of Systemic Lupus Erythematosus. Autoimmune Dis 2012, 2012, 834291. https://doi.org/10.1155/2012/834291.
(4) Zayat, A. S.; Md Yusof, M. Y.; Wakefield, R. J.; Conaghan, P. G.; Emery, P.; Vital, E. M. The Role of Ultrasound in Assessing Musculoskeletal Symptoms of Systemic Lupus Erythematosus: A Systematic Literature Review. Rheumatology (Oxford) 2016, 55 (3), 485-494. https://doi.org/10.1093/rheumatology/kev343.
(5) Sa, C.; E, A.; A, R.; D, I. Damage and mortality in a group of British patients with systemic lupus erythematosus followed up for over 10 years https://pubmed.ncbi.nlm.nih.gov/19359343/(accessed Feb. 8, 2021). https://doi.org/10.1093/rheumatology/kep062.
(6) Murimi-Worstell, I. B.; Lin, D. H.; Nab, H.; Kan, H. J.; Onasanya, O.; Tierce, J. C.; Wang, X.; Desta, B.; Alexander, G. C.; Hammond, E. R. Association between Organ Damage and Mortality in Systemic Lupus Erythematosus: A Systematic Review and Meta-Analysis. BMJ Open 2020, 10 (5), e031850. https://doi.org/10.1136/bmjopen-2019-031850.
(7) Doria, A.; Briani, C. Lupus: Improving Long-Term Prognosis. Lupus 2008, 17 (3), 166-170. https://doi.org/10.1177/0961203307087612.
(8) Petri, M. Long-Term Outcomes in Lupus. Am J Manag Care 2001, 7 (16 Suppl), S480-485.
(9) Zonana-Nacach, A.; Barr, S. G.; Magder, L. S.; Petri, M. Damage in Systemic Lupus Erythematosus and Its Association with Corticosteroids. Arthritis Rheum 2000, 43 (8), 1801-1808. https://doi.org/10.1002/1529-0131 (200008) 43:8<1801: AID-ANR16>3.0.CO;2-O.
(10) Urowitz, M. B.; Bookman, A. A.; Koehler, B. E.; Gordon, D. A.; Smythe, H. A.; Ogryzlo, M. A. The Bimodal Mortality Pattern of Systemic Lupus Erythematosus. Am J Med 1976, 60 (2), 221-225. https://doi.org/10.1016/0002-9343 (76) 90431-9.
(11) Patel, D. D.; Antoni, C.; Freedman, S. J.; Levesque, M. C.; Sundy, J. S. Phase 2 to Phase 3 Clinical Trial Transitions: Reasons for Success and Failure in Immunologic Diseases. Journal of Allergy and Clinical Immunology 2017, 140 (3), 685-687. https://doi.org/10.1016/j.jaci.2017.04.029.
(12) Dowden, H.; Munro, J. Trends in Clinical Success Rates and Therapeutic Focus. Nature Reviews Drug Discovery 2019, 18 (7), 495-496. https://doi.org/10.1038/d41573-019-00074-z.
(13) Eisenberg, R. WHY CAN'T WE FIND A NEW TREATMENT FOR SLE? J Autoimmun 2009, 32 (3-4), 223-230. https://doi.org/10.1016/j.jaut.2009.02.006.
(14) Isenberg, D. A.; Petri, M.; Kalunian, K.; Tanaka, Y.; Urowitz, M. B.; Hoffman, R. W.; Morgan-Cox, M.; likuni, N.; Silk, M.; Wallace, D. J. Efficacy and Safety of Subcutaneous Tabalumab in Patients with Systemic Lupus Erythematosus: Results from ILLUMINATE-1, a 52-Week, Phase III, Multicentre, Randomised, Double-Blind, Placebo-Controlled Study. Ann Rheum Dis 2016, 75 (2), 323-331. https://doi.org/10.1136/annrheumdis-2015-207653.
(15) Isenberg, D.; Merrill, J.; Hoffman, R.; Linnik, M.; Morgan-Cox, M.; Veenhuizen, M.; likuni, N.; Dickson, C.; Silk, M.; Wallace, D.; Dörner, T. OP0184 Efficacy and Safety of Tabalumab in Patients with Systemic Lupus Erythematosus (SLE): Results from 2 Phase 3, 52-Week, Multicenter, Randomized, Placebo-Controlled Trials. Annals of the Rheumatic Diseases 2015, 74 (Suppl 2), 141-141. https://doi.org/10.1136/annrheumdis-2015-eular. 1195.
(16) Furie, R. A.; Leon, G.; Thomas, M.; Petri, M. A.; Chu, A. D.; Hislop, C.; Martin, R. S.; Scheinberg, M. A.; PEARL-SC Study. A Phase 2, Randomised, Placebo-Controlled Clinical Trial of Blisibimod, an Inhibitor of B Cell Activating Factor, in Patients with Moderate-to-Severe Systemic Lupus Erythematosus, the PEARL-SC Study. Ann Rheum Dis 2015, 74 (9), 1667-1675. https://doi.org/10.1136/annrheumdis-2013-205144.
(17) Merrill, J. T.; Shanahan, W. R.; Scheinberg, M.; Kalunian, K. C.; Wofsy, D.; Martin, R. S. Phase III Trial Results with Blisibimod, a Selective Inhibitor of B-Cell Activating Factor, in Subjects with Systemic Lupus Erythematosus (SLE): Results from a Randomised, Double-Blind, Placebo-Controlled Trial. Annals of the Rheumatic Diseases 2018, 77 (6), 883-889. https://doi.org/10.1136/annrheumdis-2018-213032.
(18) Isenberg, D.; Gordon, C.; Licu, D.; Copt, S.; Rossi, C. P.; Wofsy, D. Efficacy and Safety of Atacicept for Prevention of Flares in Patients with Moderate-to-Severe Systemic Lupus Erythematosus (SLE): 52-Week Data (APRIL-SLE Randomised Trial). Ann Rheum Dis 2015, 74 (11), 2006-2015. https://doi.org/10.1136/annrheumdis-2013-205067.
(19) Alarcón-Segovia, D.; Tumlin, J. A.; Furie, R. A.; Mckay, J. D.; Cardiel, M. H.; Strand, V.; Bagin, R. G.; Linnik, M. D.; Hepburn, B.; LJP 394 Investigator Consortium. LJP 394 for the Prevention of Renal Flare in Patients with Systemic Lupus Erythematosus: Results from a Randomized, Double-Blind, Placebo-Controlled Study. Arthritis Rheum 2003, 48 (2), 442-454. https://doi.org/10.1002/art. 10763.
(20) Mahieu, M. A.; Strand, V.; Simon, L. S.; Lipsky, P. E.; Ramsey-Goldman, R. A Critical Review of Clinical Trials in Systemic Lupus Erythematosus. Lupus 2016, 25 (10), 1122-1140. https://doi.org/10.1177/0961203316652492.
(21) Rovin, B. H.; Furie, R.; Latinis, K.; Looney, R. J.; Fervenza, F. C.; Sanchez-Guerrero, J.; Maciuca, R.; Zhang, D.; Garg, J. P.; Brunetta, P.; Appel, G.; LUNAR Investigator Group. Efficacy and Safety of Rituximab in Patients with Active Proliferative Lupus Nephritis: The Lupus Nephritis Assessment with Rituximab Study. Arthritis Rheum 2012, 64 (4), 1215-1226. https://doi.org/10.1002/art.34359.
(22) Merrill, J. T.; Neuwelt, C. M.; Wallace, D. J.; Shanahan, J. C.; Latinis, K. M.; Oates, J. C.; Utset, T. O.; Gordon, C.; Isenberg, D. A.; Hsieh, H.-J.; Zhang, D.; Brunetta, P. G. Efficacy and Safety of Rituximab in Moderately-to-Severely Active Systemic Lupus Erythematosus: The Randomized, Double-Blind, Phase II/III Systemic Lupus Erythematosus Evaluation of Rituximab Trial. Arthritis Rheum 2010, 62 (1), 222-233. https://doi.org/10.1002/art.27233.
(23) Daikh, D. I.; Wofsy, D. Cutting Edge: Reversal of Murine Lupus Nephritis with CTLA4lg and Cyclophosphamide. The Journal of Immunology 2001, 166 (5), 2913-2916. https://doi.org/10.4049/jimmunol. 166.5.2913.
(24) Merrill, J. T.; Burgos-Vargas, R.; Westhovens, R.; Chalmers, A.; D′Cruz, D.; Wallace, D. J.; Bae, S. C.; Sigal, L.; Becker, J.-C.; Kelly, S.; Raghupathi, K.; Li, T.; Peng, Y.; Kinaszczuk, M.; Nash, P. The Efficacy and Safety of Abatacept in Patients with Non-Life-Threatening Manifestations of Systemic Lupus Erythematosus: Results of a Twelve-Month, Multicenter, Exploratory, Phase Ilb, Randomized, Double-Blind, Placebo-Controlled Trial. Arthritis Rheum 2010, 62 (10), 3077-3087. https://doi.org/10.1002/art.27601.
(25) Strand, V.; Petri, M.; Kalunian, K.; Gordon, C.; Wallace, D. J.; Hobbs, K.; Kelley, L.; Kilgallen, B.; Wegener, W. A.; Goldenberg, D. M. Epratuzumab for Patients with Moderate to Severe Flaring SLE: Health-Related Quality of Life Outcomes and Corticosteroid Use in the Randomized Controlled ALLEVIATE Trials and Extension Study SL0006. Rheumatology (Oxford) 2014, 53 (3), 502-511. https://doi.org/10.1093/rheumatology/ket378.
(26) Wallace, D. J.; Kalunian, K.; Petri, M. A.; Strand, V.; Houssiau, F. A.; Pike, M.; Kilgallen, B.; Bongardt, S.; Barry, A.; Kelley, L.; Gordon, C. Efficacy and Safety of Epratuzumab in Patients with Moderate/Severe Active Systemic Lupus Erythematosus: Results from EMBLEM, a Phase IIb, Randomised, Double-Blind, Placebo-Controlled, Multicentre Study. Ann Rheum Dis 2014, 73 (1), 183-190. https://doi.org/10.1136/annrheumdis-2012-202760.
(27) Wallace, D. J.; Strand, V.; Merrill, J. T.; Popa, S.; Spindler, A. J.; Eimon, A.; Petri, M.; Smolen, J. S.; Wajdula, J.; Christensen, J.; Li, C.; Diehl, A.; Vincent, M. S.; Beebe, J.; Healey, P.; Sridharan, S. Efficacy and Safety of an Interleukin 6 Monoclonal Antibody for the Treatment of Systemic Lupus Erythematosus: A Phase II Dose-Ranging Randomised Controlled Trial. Annals of the Rheumatic Diseases 2017, 76 (3), 534-542. https://doi.org/10.1136/annrheumdis-2016-209668.
(28) Blair, H. A.; Duggan, S. T. Belimumab: A Review in Systemic Lupus Erythematosus. Drugs 2018, 78 (3), 355-366. https://doi.org/10.1007/s40265-018-0872-z.
(29) Navarra, S. V.; Guzmán, R. M.; Gallacher, A. E.; Hall, S.; Levy, R. A.; Jimenez, R. E.; Li, E. K.-M.; Thomas, M.; Kim, H.-Y.; León, M. G.; Tanasescu, C.; Nasonov, E.; Lan, J.-L.; Pineda, L.; Zhong, Z. J.; Freimuth, W.; Petri, M. A.; BLISS-52 Study Group. Efficacy and Safety of Belimumab in Patients with Active Systemic Lupus Erythematosus: A Randomised, Placebo-Controlled, Phase 3 Trial. Lancet 2011, 377 (9767), 721-731. https://doi.org/10.1016/S0140-6736 (10) 61354-2.
(30) Furie, R.; Petri, M.; Zamani, O.; Cervera, R.; Wallace, D. J.; Tegzová, D.; Sanchez-Guerrero, J.; Schwarting, A.; Merrill, J. T.; Chatham, W. W.; Stohl, W.; Ginzler, E. M.; Hough, D. R.; Zhong, Z. J.; Freimuth, W.; van Vollenhoven, R. F.; BLISS-76 Study Group. A Phase III, Randomized, Placebo-Controlled Study of Belimumab, a Monoclonal Antibody That Inhibits B Lymphocyte Stimulator, in Patients with Systemic Lupus Erythematosus. Arthritis Rheum 2011, 63 (12), 3918-3930. https://doi.org/10.1002/art.30613.
(31) van Vollenhoven, R. F.; Petri, M.; Wallace, D. J.; Roth, D. A.; Molta, C. T.; Hammer, A. E.; Tang, Y.; Thompson, A. Cumulative Corticosteroid Dose Over Fifty-Two Weeks in Patients With Systemic Lupus Erythematosus: Pooled Analyses From the Phase III Belimumab Trials. Arthritis Rheumatol 2016, 68 (9), 2184-2192. https://doi.org/10.1002/art.39682.
(32) Morand, E. F.; Furie, R.; Tanaka, Y.; Bruce, I. N.; Askanase, A. D.; Richez, C.; Bae, S.-C.; Brohawn, P. Z.; Pineda, L.; Berglind, A.; Tummala, R. Trial of Anifrolumab in Active Systemic Lupus Erythematosus. New England Journal of Medicine 2020, 382 (3), 211-221. https://doi.org/10.1056/NEJMoa1912196.
(33) Furie, R.; Khamashta, M.; Merrill, J. T.; Werth, V. P.; Kalunian, K.; Brohawn, P.; Illei, G. G.; Drappa, J.; Wang, L.; Yoo, S.; Investigators, for the C. S. Anifrolumab, an Anti-Interferon-α Receptor Monoclonal Antibody, in Moderate-to-Severe Systemic Lupus Erythematosus. Arthritis & Rheumatology (Hoboken, N.j.) 2017, 69 (2), 376. https://doi.org/10.1002/art.39962.
(34) Furie, R. A.; Morand, E. F.; Bruce, I. N.; Manzi, S.; Kalunian, K. C.; Vital, E. M.; Ford, T. L.; Gupta, R.; Hiepe, F.; Santiago, M.; Brohawn, P. Z.; Berglind, A.; Tummala, R. Type I Interferon Inhibitor Anifrolumab in Active Systemic Lupus Erythematosus (TULIP-1): A Randomised, Controlled, Phase 3 Trial. The Lancet Rheumatology 2019, 1 (4), e208-e219. https://doi.org/10.1016/S2665-9913 (19) 30076-1.
(35) Isenberg, D. A.; Merrill, J. T. Why, Why, Why de-Lupus (Does so Badly in Clinical Trials). Expert Rev Clin Immunol 2016, 12 (2), 95-98. https://doi.org/10.1586/1744666X.2016.1112270.
(36) Wallace, D. J. The Evolution of Drug Discovery in Systemic Lupus Erythematosus. Nat Rev Rheumatol 2015, 11 (10), 616-620. https://doi.org/10.1038/nrrheum.2015.86.
(37) Felten, R.; Sagez, F.; Gavand, P.-E.; Martin, T.; Korganow, A.-S.; Sordet, C.; Javier, R.-M.; Soulas-Sprauel, P.; Rivière, M.; Scher, F.; Poindron, V.; Guffroy, A.; Arnaud, L. 10 Most Important Contemporary Challenges in the Management of SLE. Lupus Science & Medicine 2019, 6 (1), e000303. https://doi.org/10.1136/lupus-2018-000303.
(38) Yao, Y.; Higgs, B. W.; Richman, L.; White, B.; Jallal, B. Use of Type | Interferon-Inducible MRNAs as Pharmacodynamic Markers and Potential Diagnostic Markers in Trials with Sifalimumab, an Anti-IFNa Antibody, in Systemic Lupus Erythematosus. Arthritis Res Ther 2010, 12 (Suppl 1), S6. https://doi.org/10.1186/ar2887.
(39) A Pilot Study Characterizing Factors in Adherence to Cutaneous Lupus Treatment-Teresa Ro, Rechelle Z. Tull, Leah A. Cardwell, Michael E. Farhangian, Farah Moustafa, Irma Richardson, Joseph L. Jorizzo, Steven Feldman, William Huang, 2018 https://journals.sagepub.com/doi/abs/10.1177/1203475418770581 (accessed Apr. 12, 2021).
(40) Shi, H.; Gudjonsson, J. E.; Kahlenberg, J. M. Treatment of Cutaneous Lupus Erythematosus: Current Approaches and Future Strategies. Current Opinion in Rheumatology 2020, 32 (3), 208-214. https://doi.org/10.1097/BOR.0000000000000704.
(41) Tummala, R.; Rouse, T.; Berglind, A.; Santiago, L. Safety, Tolerability and Pharmacokinetics of Subcutaneous and Intravenous Anifrolumab in Healthy Volunteers. Lupus Sci Med 2018, 5 (1), e000252. https://doi.org/10.1136/lupus-2017-000252.
(42) Ferguson, G. T.; Mansur, A. H.; Jacobs, J. S.; Hebert, J.; Clawson, C.; Tao, W.; Wu, Y.; Goldman, M. Assessment of an Accessorized Pre-Filled Syringe for Home-Administered Benralizumab in Severe Asthma. J Asthma Allergy 2018, 11, 63-72. https://doi.org/10.2147/JAA.S157762.
(43) Albrecht, J.; Taylor, L.; Berlin, J. A.; Dulay, S.; Ang, G.; Fakharzadeh, S.; Kantor, J.; Kim, E.; Militello, G.; McGinnis, K.; Richardson, S.; Treat, J.; Vittorio, C.; Van Voorhees, A.; Werth, V. P. The CLASI (Cutaneous Lupus Erythematosus Disease Area and Severity Index): An Outcome Instrument for Cutaneous Lupus Erythematosus. J Invest Dermatol 2005, 125 (5), 889-894. https://doi.org/10.1111/j.0022-202X.2005.23889.x.
(44) Klein, R. S.; Morganroth, P. A.; Werth, V. P. Cutaneous Lupus and the CLASI Instrument. Rheum Dis Clin North Am 2010, 36 (1), 33-51. https://doi.org/10.1016/j.rdc.2009.12.001.
(45) Touma, Z.; Urowitz, M.; Gladman, D. SLEDAI-2K for a 30-Day Window. Lupus 2010, 19 (1), 49-51. https://doi.org/10.1177/0961203309346505.
(46) Interferon-Inducible Gene Expression Kit As a Potential Diagnostic Test for Anifrolumab: Analytical Validation for Use in Clinical Trials. ACR Meeting Abstracts.
(47) Doria, A.; Gatto, M.; laccarino, L.; Punzi, L. Value and Goals of Treat-to-Target in Systemic Lupus Erythematosus: Knowledge Foresight. Lupus 2015, 24 and (4-5), 507-515. https://doi.org/10.1177/0961203314559087.
(48) Karp, I.; Abrahamowicz, M.; Fortin, P. R.; Pilote, L.; Neville, C.; Pineau, C. A.; Esdaile, J. M. Recent Corticosteroid Use and Recent Disease Activity: Independent Determinants of Coronary Heart Disease Risk Factors in Systemic Lupus Erythematosus? Arthritis Rheum 2008, 59 (2), 169-175. https://doi.org/10.1002/art.23352.

1. A method for steroid-sparing in a subject in need thereof, comprising administering to the subject a therapeutically effective amount of a type I IFN receptor (IFNAR1) inhibitor and a steroid, wherein the dose of the steroid administered to the subject is tapered from a pre-sparing dose at baseline to a post-sparing dose, wherein the subject has systemic lupus erythematosus (SLE).

2. The method of claim 1, wherein the method does not worsen SLE disease activity in the subject.

3. The method of any preceding claim, wherein the post-sparing dose is ≤75% of the pre-sparing dose; is ≤50% of the pre-sparing dose; is ≤25% of the pre-sparing dose; or is ≤10% of the pre-sparing dose.

4-8. (canceled)

9. The method of claim 1, wherein the pre-sparing steroid dose is about ≥10 mg/day prednisone or prednisone-equivalent dose.

10. (canceled)

11. The method of claim 9, wherein the post-sparing steroid dose is about ≤5 mg/day prednisone or prednisone-equivalent dose.

12. The method of claim 1, wherein the post-sparing dose is maintained for ≥12 weeks.

13-14. (canceled)

15. The method of claim 1, wherein the post-sparing dose is about 0 mg/day prednisone or prednisone-equivalent dose.

16. The method of claim 1, wherein the post-sparing dose is sustained for at least 1 week.

17-27. (canceled)

28. The method of any claim 1, wherein the steroid comprises a glucocorticoid, optionally wherein the steroid comprises an oral glucocorticoid.

29. The method of claim 1, wherein the steroid comprises hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof.

30. The method of claim 1, wherein the steroid comprises prednisone.

31-45. (canceled)

46. The method of claim 1, wherein the IFNAR1 inhibitor is a human monoclonal antibody specific for IFNAR1, optionally a modified IgG1 class human monoclonal antibody.

47. The method of claim 46, wherein the antibody comprises:
a) a heavy chain variable region complementarity determining region 1 (HCDR1) comprising the amino acid sequence of SEQ ID NO: 3;
b) a heavy chain variable region complementarity determining region 2 (HCDR2) comprising the amino acid sequence of SEQ ID NO: 4;
c) a heavy chain variable region complementarity determining region 3 (HCDR3) comprising the amino acid sequence of SEQ ID NO: 5;
d) a light chain variable region complementarity determining region 1 (LCDR1) comprising the amino acid sequence SEQ ID NO: 6;
e) a light chain variable region complementarity determining region 2 (LCDR2) comprising the amino acid sequence SEQ ID NO: 7; and
f) a light chain variable region complementarity determining region 3 (LCDR3) comprising the amino acid sequence SEQ ID NO: 8.

48. The method of claim 46, wherein the antibody comprises in the Fc region an amino acid substitution of L234F, as numbered by the EU index as set forth in Kabat and wherein said antibody exhibits reduced affinity for at least one Fc ligand compared to an unmodified antibody.

49. The method of claim 46, wherein the antibody comprises:
a) a human heavy chain variable region comprising the amino acid sequence of SEQ ID NO: 1;
b) a human light chain variable region comprising the amino acid sequence of SEQ ID NO: 2.

50. The method of claim 46, wherein the antibody comprises:
a) a human light chain constant region comprising the amino acid sequence of SEQ ID NO: 9; and
b) a human heavy chain constant region comprising the amino acid sequence of SEQ ID NO: 10.

51. The method of claim 1, wherein the IFNAR1 inhibitor is anifrolumab or a functional variant thereof.

52. (canceled)

53. The method of claim 51, wherein the method comprises administering about 300 mg to about 1000 mg of anifrolumab or the functional variant thereof.

54. (canceled)

55. The method of claim 51, comprising administering anifrolumab or the functional variant thereof at a dose of 300-1000 mg every four weeks (Q4W).

56. (canceled)

57. The method of claim 51, wherein anifrolumab or the functional variant thereof is administered subcutaneously.

58-67. (canceled)


SEQUENCE LISTING (24 sequences)

SEQ ID NO: 1 - protein, 117 aa, Artificial Sequence [sequence omitted]

SEQ ID NO: 2 - protein, 108 aa, Artificial Sequence [sequence omitted]

SEQ ID NO: 3 - protein, 5 aa, Artificial Sequence
  HCDR1
NYWIA

SEQ ID NO: 4 - protein, 17 aa, Artificial Sequence
  HCDR2
IIYPGDSDIRYSPSFQG

SEQ ID NO: 5 - protein, 8 aa, Artificial Sequence
  HCDR3
HDIEGFDY

SEQ ID NO: 6 - protein, 12 aa, Artificial Sequence
  LCDR1
RASQSVSSSFFA

SEQ ID NO: 7 - protein, 7 aa, Artificial Sequence
  LCDR2
GASSRAT

SEQ ID NO: 8 - protein, 9 aa, Artificial Sequence
  LCDR3
QQYDSSAIT

SEQ ID NO: 9 - protein, 107 aa, Artificial Sequence [sequence omitted]

SEQ ID NO: 10 - protein, 330 aa, Artificial Sequence
  Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 440 aa, Artificial Sequence
  Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKS

SEQ ID NO: 12 - protein, 215 aa, Artificial Sequence
  Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 80 aa, Artificial Sequence
  H15D10 (VH)
EVQLVQSGAEVKKPGESLRISCKGSGYTFTNYWVAWVRQMPGKGLESMGIIYPGDSDTRYSPSFQGHVTISADKSISTAY

SEQ ID NO: 14 - protein, 108 aa, Artificial Sequence
  L8C3 (VL)
DIQMTQSPSSLSASLGDRVTITCRASQNVGNYLNWYQQKPGKAPKLLIYRASNLASGVPSRFSGSGSGTDFTLTISSLQPEDFATYYCQQMEHAPPTFGQGTKVEIKR

SEQ ID NO: 15 - protein, 108 aa, Artificial Sequence
  L16C11 (VL)
EIVLTQSPGTLSLSPGERATLSCRASQSVIGYYLAWYQQKPGQAPRLLIYSVSTLASGIPDRFSGSGSGTDFTLTISRLEPEDFAVYYCQQYYRFPITFGQGTKVEIK

SEQ ID NO: 16 - protein, 117 aa, Artificial Sequence
  H19B7 (VH)
EVQLVQSGAEVKKPGESLRISCKGSGYTFTNYWMAWVRQMPGKGLESMGIIYPSDSDTRYSPSFQGHVTISADKSISTAYLQWSSLKASDTAMYYCARHDVEGYDYWGQGTLVTVSS

SEQ ID NO: 17 - protein, 119 aa, Artificial Sequence
  QX006N (VH)
EVQLVESGGGLVQPGGSLRLSCAASGFSLSSYYMTWVRQAPGKGLEWVSVINVYGGTYYASWAKGRFTISRDNSKNTLYLQMNSLRAEDTAVYYCAREDVAVYMAIDLWGQGTLVTVSS

SEQ ID NO: 18 - protein, 111 aa, Artificial Sequence
  QX006N (VL)
AIQMTQSPSSLSASVGDRVTITCQASQSISNQLSWYQQKPGKAPKLLIYDASSLASGVPSRFSGSRSGTKFTLTISSLQPEDFATYYCLGIYGDGADDGIAFGGGTKVEIK

SEQ ID NO: 19 - protein, 5 aa, Artificial Sequence
  QX006N (HCDR1)
SYYMT

SEQ ID NO: 20 - protein, 16 aa, Artificial Sequence
  QX006N (HCDR2)
VINVYGGTYYASWAKG

SEQ ID NO: 21 - protein, 11 aa, Artificial Sequence
  QX006N (HCDR3)
EDVAVYMAIDL

SEQ ID NO: 22 - protein, 11 aa, Artificial Sequence
  QX006N (LCDR1)
QASQSISNQLS

SEQ ID NO: 23 - protein, 7 aa, Artificial Sequence
  QX006N (LCDR2)
DASSLAS

SEQ ID NO: 24 - protein, 13 aa, Artificial Sequence
  QX006N (LCDR3)
LGIYGDGADDGIA